(19) United States
(12) Patent Application Publication
(10) Pub. No.: US 2026/0259181 A1
(43) Pub. Date: Sep. 3, 2026

(54) EXHALED BREATH CAPTURE MODULES AND SUBSTRATE-BASED PROBES FOR MONITORING PROTEOLYSIS ACTIVITY

(71) Applicant: Zeteo Tech, Inc., Sykesville, MD (US)

(72) Inventors: Dapeng CHEN, Sykesville, MD (US); Wayne A. BRYDEN, Sykesville, MD (US); Michael MCLOUGHLIN, Sykesville, MD (US); Emily R. CATON, Reisterstown, MD (US); Kiana M. KISER, Hanover, MD (US); Caroline R. HADDAWAY, Annapolis, MD (US); Maximilian Schrier CETTA, Potomac, MD (US)

(73) Assignee: Zeteo Tech, Inc., Sykesville, MD (US)

(21) Appl. No.: 19/652,788

(22) Filed: Apr. 20, 2026

Related U.S. Application Data
(63) Continuation-in-part of application No. PCT/US2025/054226, filed on Nov. 5, 2025.
(60) Provisional application No. 63/716,602, filed on Nov. 5, 2024.
(60) Provisional application No. 63/734,970, filed on Dec. 17, 2024.

(51) Int. Cl. G01N 30/14; A61B 10/00; B01D 53/04; C12Q 1/689; G01N 30/52; G01N 30/72

(52) U.S. Cl. CPC G01N 30/14; B01D 53/0423; B01D 53/0446; C12Q 1/689; G01N 30/52; G01N 30/72; A61B 2010/0087; B01D 2253/106; B01D 2253/202; B01D 2253/304; B01D 2257/91; B01D 2259/4533; B01D 2259/4541; G01N 2030/521

(57) ABSTRACT

Methods and devices for capturing aerosolized organic biomaterials including proteases in exhaled breath from patients with respiratory tract infections who are inhaling supplemental oxygen using a nasal cannula, and without causing discomfort to the patients. The patients may inhale supplemental oxygen supplied by a nasal cannula and exhale into an exhaled breath capture element configured to impose minimal to no back pressure on the patient. The exhaled breath capture elements include an activated packed bed column. Methods and devices for examining the exhaled breath samples for proteolysis activity using a substrate-based probe or a plurality of substrate based probes and MALDI-TOFMS to detect LRTIs. Sequential methods including examining proteolysis activity to predict the presence of LRTI followed by a qPCR assay, may provide pathogen-specific information, and offer a comprehensive solution for managing LRTI cases.

### RELATED APPLICATIONS

This patent application is a continuation-in-part application claiming priority to International Patent Application No. PCT/US2025/054226 entitled “SUBSTRATE-BASED PROBES FOR MONITORING PROTEASE ACTIVITY USING MALDI-TOFMS,” and filed on Nov. 5, 2025, which claims priority to U.S. Provisional Pat. Appl. No. 63/716,602 entitled “SUBSTRATE-BASED PROBES FOR MONITORING PROTEASE ACTIVITY USING MALDI-TOFMS,” and filed on Nov. 5, 2024, to U.S. Provisional Pat. Appl. No. 63/734,970 entitled “SUBSTRATE-BASED PROBES FOR MONITORING PROTEASE ACTIVITY USING MALDI-TOFMS,” and filed on Dec. 17, 2024. This patent application is related to International Patent Application No. PCT/US2024/038023 entitled “SUBSTRATE-BASED PROBES FOR MONITORING PROTEASE ACTIVITY USING MALDI-TOFMS,” filed on Jul. 15, 2024, which claims priority to U.S. Provisional Pat. Appl. No. 63/526,925 entitled “SUBSTRATE-BASED PROBES FOR MONITORING PROTEASE ACTIVITY USING MALDI-TOFMS,” and filed on Jul. 14, 2023, and to U.S. Provisional Pat. Appl. No. 63/545,697 entitled “SUBSTRATE-BASED PROBES FOR MONITORING PROTEASE ACTIVITY USING MALDI-TOFMS,” and filed on Oct. 25, 2023, all of which are assigned to the assignee hereof. The disclosures of all prior applications are considered part of and are incorporated by reference in this patent application in their respective entireties.

### FEDERALLY SPONSORED RESEARCH AND DEVELOPMENT

None.

### TECHNICAL FIELD

This disclosure relates to methods and devices for capturing aerosolized non-volatile organic biomaterials including proteases in exhaled air using packed bed columns installed in exhaled breath capture modules for capturing exhaled breath samples from patients with respiratory tract infections who are inhaling supplemental oxygen using a nasal cannula, and without causing discomfort to the patients. More particularly, but not by way of limitation, the present disclosure relates to methods and devices for capturing one or more proteases in exhaled breath using low-back pressure exhaled breath capture modules and examining proteolysis activity using one or more substrate-based probes and MALDI-TOFMS to detect lower respiratory tract infections (“LRTIs”). Causative pathogens associated with the LRTIs may be identified using a qPCR assay.

### BACKGROUND

Exhaled air aerosols (also referred to herein as “exhaled breath aerosols”) contain non-volatile organic biomarkers produced by human biological processes, including metabolic, immunological, and inflammatory processes, and the composition of these compounds may be viewed as indicators for human health. The detection of these protein biomarkers using analysis of exhaled air may be used to monitor, screen, diagnose, and distinguish between healthy persons and persons with health issues such as obesity, diabetes, liver cancer, lung cancer, and the like. The capture of these biomarkers from exhaled breath and subsequent analysis may reveal risk factors and assist with diagnosis, treatment, and mitigation of the spread of diseases.

Although research has shown that respiratory diseases can be detected from breath aerosol and breath condensate, modern clinical tests for infections or diseases such as COVID-19, tuberculosis, influenza, pneumonia continue to utilize sputum, blood, or nasal swabs. Coronavirus Disease (“COVID-19”) is a disease caused by the newly emerged coronavirus SARS-COV-2. This new coronavirus is a respiratory virus and spreads primarily through droplets generated when an infected person coughs or sneezes, or through droplets of saliva or discharge from the nose. The novel coronavirus is highly contagious and has recently created a pandemic. Additionally, tuberculosis (“TB”) has surpassed HIV/AIDS as a global killer with more than 4000 daily deaths. (Patterson, B., et al., 2018). In communities with highly prevalent HIV, Mycobacterium tuberculosis (Mtb) genotyping studies have found that recent transmission, rather than reactivation, accounts for the majority (54%) of incident TB cases. The physical process of TB transmission remains poorly understood and the application of new technologies to elucidate key events in infectious aerosol production, release, and inhalation, has been slow. Interruption of transmission would likely have a rapid, measurable impact on TB incidence. To mitigate transmission of respiratory diseases, rapid disease detection tools are needed.

The time associated with a diagnostic assay is a critical parameter for a fielded, or “point of care” test. Active Case Finding (“ACF”) is an example of a fielded diagnostic assay because, by definition, ACF takes place outside the healthcare system. According to the World Health Organization, ACF is a “systematic identification of people with suspected active TB, using tests, examinations, or other procedures that can applied rapidly.” In the U.S., a point-of-care test is required to provide an answer in preferably 20 minutes or less. The GeneXpert assay (Cepheid, Inc., Sunnyvale, CA) may be used to provide diagnosis in about one hour. The GeneXpert genetic assay is based on polymerase chain reaction (“PCR”) and may be used to analyze a sample for respiratory disease diagnosis. This assay is expensive to implement on a “cost per test” basis, and therefore it is not yet widely deployed. Because of its high cost, in developing countries, it is not used to screen patients who appear healthy (or referred to as non-symptomatic patients) but might have TB infection. Instead, it is used to confirm a diagnosis made based on other tests or factors. The goal of ACF is to get those infected to treatment as soon as possible, thereby reducing the average period of infection and the spread of the disease. In the case of TB, by the time an individual goes to a clinic for help, that person may have transmitted the infection to between about 10 other people and about 115 other people. ACF can help to reduce or prevent significant TB transmission. The diagnostic systems and methods such as sputum analysis and blood analysis are either not automated and autonomously operated or are not rapid. Many other tests include expensive assays with reagents that are consumed for each analysis, and thus, do not have general utility for active case finding, particularly in developing and underdeveloped countries.

There is increasing interest in new diagnostic tools for diseases, including respiratory diseases, using exhaled air. Exhaled breath aerosols (“EBA”) and vapors can be collected noninvasively and analyzed for characteristics to elucidate physiologic and pathologic processes in the lung. (Hunt, 2002). EBA analysis appears to be a compelling diagnostic tool for TB detection and enables rapid analysis, portability, and low cost because the need for expensive assays and consumables is eliminated. To capture exhaled breath aerosols, exhaled air is passed through a condensing apparatus to produce an accumulation of fluid that is referred to as exhaled breath condensate (“EBC”). Although predominantly derived from water vapor, EBC includes nonvolatile compounds, including cytokines, lipids, surfactants, ions, oxidation products, adenosine, histamine, acetylcholine, and serotonin. In addition, EBC includes potentially volatile water-soluble compounds, including ammonia, hydrogen peroxide, ethanol, and other volatile organic compounds. EBC may be characterized by a readily measurable pH. EBC contains aerosolized airway lining fluid and volatile compounds that provide noninvasive indications of ongoing biochemical and inflammatory activities in the lung. Increased interest in EBC has resulted from the recognition that in lung disease, EBC has measurable characteristics that can be used to differentiate between infected and healthy individuals. These assays have provided evidence of airway and lung redox deviation, acid-base status, and the degree and type of inflammation in acute and chronic asthma, chronic obstructive pulmonary disease, adult respiratory distress syndrome, occupational diseases, and cystic fibrosis. Characterized by uncertain and variable degrees of dilution, EBC may not provide precise assessment of individual solute concentrations within the native airway lining fluid.

Patterson et al. (2018) used a respiratory aerosol sampling chamber (“RASC”) designed to optimize exhaled breath aerosol sampling, and to isolate and accumulate respirable aerosol from a single patient. Environmental sampling detects Mtb present after a period of ageing in the chamber air. 35 newly diagnosed, GeneXpert sputum-positive, TB patients were monitored during confinement for about one hour in the RASC chamber which has a volume of about 1.4 m³. The chamber incorporated aerodynamic particle size detection, sampling devices, real-time CO₂ monitoring, and cough sound-recording. Microbiological culture and droplet digital polymerase chain reaction (ddPCR) were used to detect Mtb in samples collected from each of the bio-aerosol collection devices. Mtb was detected in 77% of aerosol samples and 42% of samples were positive by mycobacterial culture and 92% were positive by ddPCR. A correlation was found between cough rate and culturable bioaerosol. Mtb was detected in bioaerosols exhaled by a majority of the untreated TB-patients using the RASC chamber. Exhaled breath analytical tools have not been commercialized for ACF because methods and devices to efficiently collect and concentrate the trace amounts of analyte present in exhaled breath are lacking. Furthermore, there is no standard or methodology to assess how much exhaled breath is sufficient for a particular diagnosis.

The lack of non-invasive methods and reliable molecular biomarkers is a significant barrier to diagnosing respiratory tract infections (“RTI”) in critical care settings, especially in patients breathing using mechanical ventilators. Current diagnostic methods rely on non-specific clinical observations, such as tracheal secretions, chest X-ray findings, body temperature, white blood cell counting, oxygenation, and microbiological testing. Score systems, such as clinical pulmonary infection score (“CPIS”), have been developed based on these clinical symptoms. Although the clinical notes and score systems may be used to determine antibiotic treatment, they generally lack sensitivity and specificity for RTI diagnosis, making it challenging for clinicians to provide rational clinical decisions. Quantitative microbial culture of specimens collected from the lower respiratory tract, such as the non-invasive endotracheal aspirate (“ETA”), have been used for RTI diagnosis but are unable to inform whether the identified bacteria result from common respiratory tract colonization or from another infection. Bronchoalveolar lavage (“BAL”) has been used as a high-quality specimen collection technique from the lower respiratory tract for causative diagnosis in intubated patients. However, this method is invasive and cannot be performed routinely in intensive care units (“ICUs”). Due to these limitations, over 50% of patients admitted in intensive care units are treated without an appropriate diagnosis. Therefore, the difficulty of obtaining samples from the site of infection and the absence of accurate diagnostic molecular biomarkers limit current diagnostic methods, pathogen identification, and management of RTI in intubated patients. There is an urgent need to develop a non-invasive method for sampling the site of infection and discovering accurate molecular biomarkers for RTI diagnosis.

Non-invasive sampling methods enable repeated sampling without causing risks in critically ill patients so that a disease trajectory can be monitored. Direct sampling from the lower respiratory tract would offer specimens that better represent the site of infection and thus provide better specificity for diagnosis. Non-invasive sampling methods would encourage patients to enroll in clinical trials that can be beneficial to therapeutic and diagnostic research. Human breath and exhaled aerosols have the promise to be used as a non-invasive source in clinical use. Organic molecules contained in human breath and exhaled aerosols may be used to develop non-invasive methods for detecting lung disease exacerbation and infections. The organic molecules in human breath include two main types: volatile organic compounds (“VOCs”) and non-volatile organic compounds (“NOCs”). VOCs are gas molecules that can be emitted from non-biological sources, such as diets, plants, and home cleaning products, and thus lack specificity for biomarker use. On the contrary, NOCs are large molecules that exclusively originate from organisms, either humans or pathogens and thus are more suitable to be used as surrogate biomarkers. Non-invasive sampling methods targeting NOCs have been developed for use in clinical settings. McNeil et al. report use of inline heat moisture exchanger (“HME”) filters to collect proteins from patients with acute respiratory distress syndrome (“ARDS”). HME filters are a standard component installed in mechanical ventilators where exhaust air is present. It was reported that proteins could be captured on the HME filter as exhaled breath condensate emitted from lower airways. For this purpose, undiluted pulmonary edema fluid (“EF”) samples were collected, and the protein profiles acquired from EF samples were used to compare with HME fluid samples. The results showed a similar protein profile between the two types of samples and suggested that HME could be a non-invasive alternative to EF for distal sampling airspace in patients with ARDS.

HME filters have their limitations. They include sponge-like materials with hygroscopic properties. It is speculated that the capture of proteins is via condensation on the sponge type materials. During condensation, Reifart et al. (2021) reported that submicron particles such as SARS-COV-2 viruses are not efficiently collected on the filters mainly because the particles in human exhaled air are too small and less than 1 μm in size.

Since the particles in human breath and exhaled aerosols are composed of submicron particles, capturing these particles using the example devices and methods disclosed herein overcome the limitations of HME filters by collecting exhaled breath aerosol and breath condensate at high flow rate, high efficiency, and into relatively concentrated samples. Further, the disclosed example devices and methods provide for sample normalization by enabling the recording of individual CO₂ levels in exhaled breath.

Additionally, size sorting of aerosol particles may be used to increase the signal to noise ratio for specific analytes prior to collection of the analytes. The concentrated samples may then be analyzed by several methods, but preferably, using methods that are sensitive, rapid, and highly specific to the analytes of interest. Mass spectrometry, real-time PCR, and immunoassays have the highest potential to be sensitive, specific and nearly real-time. Sample collection methods are needed that can be coupled with fast diagnostic tools such as mass spectrometry (“MS”) that is more rapid and more reliable than sputum analysis and less invasive than blood analysis to provide a diagnostic assay that is fast, sensitive, specific and preferably, characterized by low cost per test. Such a system may be used for active case finding (“ACF”) of respiratory tract diseases and to monitor the status of patients who use ventilators to assist breathing in a hospital intensive care unit. To be effective, the sample collection and diagnostic system must be rapid and inexpensive on a “per diagnosis” basis. Low cost-per-test is a requirement for screening a large number of individuals to proactively prevent disease transmission to search for the few that are indeed infected. Low-cost devices and methods would also be required for point-of-care diagnosis of influenza and other pathogenic viruses because patients probably infected with a “common cold” may be infected with rhinovirus. In some cases, the respiratory infection will be driven by a bacterial or fungal microbe and may be treatable with antibiotics. In other cases, the microbe may be resistant to antibiotics, and a diagnostic method that can identify microbial resistance to antibiotics is preferable.

Rapid EBA methods for distinguishing between viral and bacterial infections in the respiratory tract are desired while minimizing the occurrence of false negatives due to an insufficient sample volume. Mass spectrometry, genomics methods including PCR, and immunoassays have the highest potential to be sensitive and specific. Mass spectrometry, and in particular, MALDI time-of-flight mass spectrometry (“MALDI-TOFMS”), is a preferred diagnostic tool for analysis EBA and EBC samples.

### BRIEF DISCLOSURE

This summary is provided to introduce in a simplified form a selection of concepts that are further described below in the Detailed Description. This Summary is not intended to identify key features or essential features of the claimed subject matter, nor is it intended to limit the scope of the claimed subject matter.

In some implementations, an exhaled breath capture module configured to capture aerosolized non-volatile organic particles in exhaled breath may include an inlet duct configured to receive a mouthpiece removably insertable into the inlet duct and an outlet port disposed opposite to the inlet duct. An outlet tubing including an inlet end and an outlet end may be removably insertable into the outlet port and extend into the inlet duct defining an annular region between the outlet tubing and the inlet duct. An exhaled breath capture element including a packed bed column configured to capture the aerosolized non-volatile organic particles in exhaled breath may be removably insertable or coupled into the inlet end of the outlet tubing defining a gap of predetermined length between an outlet of the mouthpiece and the exhaled breath capture element. In some instances, the exhaled breath capture module may include a reservoir region disposed in fluid communication with the inlet duct and the annular region and configured to collect saliva particles or breath condensate particles in the exhaled breath that drop through the gap. In some other instances, the exhaled breath capture module may include a vent disposed in fluid communication with the reservoir region. The vent may be configured to prevent pressure build-up in the exhaled breath capture module and thereby preventing any discomfort when a patient who is experiencing a difficulty in breathing is asked to breath into the exhaled breath capture module by a medical professional.

In some implementations, the outlet end of outlet tubing may be coupled to a pump configured to draw the exhaled breath through the packed bed column. In some other instances, the exhaled breath capture module may further include a HEPA filter coupled to the vent. In some implementations, the vent may be disposed in an orthogonal orientation relative to a longitudinal axis (A-A′) associated with the inlet duct.

In some implementations, the packed bed column may be activated by one or more of washing the packed bed column with one or more of isopropyl alcohol or methanol or by washing with water. In some examples, after activating the packed bed column, each of the inlet and outlet of the sample capture element may be capped to prevent drying out of the packed bed column. In some instances, the aerosolized non-volatile organic particles include one or more of metabolite biomarkers, lipid biomarkers, proteomic biomarkers, bacteria particles, or virus particles characteristic of one or more respiratory diseases.

In some implementations, the packed bed column may include solid particles including one or more of resins, cellulose, silica, agarose, or hydrated Fe₃O₄ particles. In some instances, the packed bed column may include resin beads having one or more of C18 functional groups on the surface, cellulose beads having sulfate ester functional groups on the surface, or a mixture thereof. In some other instances, the resin beads may have a diameter of at least about 20 μm.

In some implementations, the packed bed column may include a first packed bed including silica gel beads and a second packed bed including resin beads functionalized with C18 groups. The second packed bed may be disposed downstream of the first packed bed. In some instances, the silica gel beads may have an average diameter of between about 20 μm and about 500 μm. In some other instances, the resins beads functionalized with C18 groups may have an average diameter of between about 20 μm and about 500 μm.

In some implementations, the pump may be configured to draw exhaled breath through the exhaled breath capture element at a flow rate of between about 0.5 L/min and about 10 L/min.

In some other implementations, a system for diagnosing a respiratory disease may include the exhaled breath capture module as previously described herein, an extraction means configured to produce one or more liquid samples by extracting the aerosolized non-volatile organic particles from the packed bed column using a solvent, and an analytical device configured to analyze the particles in the one or more liquid samples. In some instances, the analytical device may include one or more of PCR, ELISA, rt-PCR, mass spectrometer (“MS”), MALDI-MS, ESI-MS, MALDI-TOFMS, or LC-MS/MS. In some other instances, the solvent may include one or more of acetonitrile (“ACN”), methanol, trifluoro acetic acid (“TFA”), or isopropanol (“IPA”), the remaining being water. In some examples, the solvent may include between about 50 vol.-% and about 70 vol.-% acetonitrile in water, between about 50 vol.-% and about 70 vol.-% isopropanol in water, and between about 0.05 vol.-% TFA in water.

In some implementations, a method for analyzing aerosolized non-volatile particles including proteases in exhaled breath may include providing an exhaled breath capture module as previously described herein, capturing one or more proteases (non-volatile organic particles) present in the exhaled breath using the packed bed column, generating one or more collected liquid samples by extracting the non-volatile particles including proteases from the packed bed column using a solvent, producing one or more reacted liquid samples associated with each of the one or more collected liquid samples by contacting a first aliquot of the one or more collected liquid samples with a plurality of substrate-based probes to disengage a cleavable molecule associated with each of the substrate-based probes in the plurality of substrate-based probes in response to proteolysis activity associated with the interaction between each of the substrate-based probes and the proteases, estimating a concentration of the proteases in the one or more reacted liquid samples by analyzing the one or more reacted liquid samples using MALDI-TOFMS, examining if the concentration of the one or more proteases in the one or more reacted liquid samples is equal to or greater than a cut-off threshold concentration corresponding to the proteases, and identifying a causative pathogen associated with the LRTI by analyzing a second aliquot of the one or more collected liquid samples using quantitative polymerase chain reaction (“qPCR”).

In some implementations, the cut-off threshold concentration is associated with a lower respiratory tract infection (“LRTI”). In some instances, the cut-off threshold concentration associated with the proteases may be between about 0.15 picomol (pM) and about 0.5 μM. In some other instances, the packed bed column may be activated prior to capturing the non-volatile particles including proteases.

In some implementations, the exhaled breath may be collected from a patient inhaling supplemental oxygen supplied via a cannula inserted into the patient's nostrils, allowing the patient to inhale the supplemental oxygen while exhaling into the mouthpiece associated with the exhaled breath capture module.

In some implementations, the plurality of substrate-based probes may include PEG36-Nle(O-Bzl)-Met(O)2-Oic-Abu-ACC, PEG20-PEG20-PLGLKARR, or PEG25-PEG20-KPLGLKAR. In some instances, the proteases may include one or more of human neutrophil elastase, trypsinogen, serine protease 22, Kallikreins, or matrix metalloproteinases (“MMPs”).

In some implementations, the solvent associated with the extracting operation may include one or more of acetonitrile (“ACN”), methanol, trifluoro acetic acid (“TFA”), or isopropanol (“IPA”), the remaining being water. In some instances, the solvent may include between about 50 vol.-% and about 70 vol.-% acetonitrile in water, between about 50 vol.-% and about 70 vol.-% isopropanol in water, and between about 0.05 vol.-% TFA in water.

Details of one or more implementations of the subject matter described in this disclosure are set forth in the accompanying drawings and the description below. Other features, aspects, and advantages will become apparent from the description, the drawings, and the claims. Note that the relative dimensions of the following figures may not be drawn to scale.

### BRIEF DESCRIPTION OF THE DRAWINGS

FIG. 1A shows a schematic diagram of an example exhaled breath sample collection system including a packed bed column, according to some implementations.

FIG. 1B shows a schematic diagram of an example exhaled air aerosol collection system for use with a ventilator assisting the breathing of a person, according to some implementations.

FIG. 1C shows a schematic diagram of an example subsystem configured to operate an exhaled air aerosol sample capture system connected to a ventilator, according to some implementations.

FIG. 2 shows a schematic diagram of another example exhaled air aerosol particle capture system including a packed bed column, according to some implementations.

FIGS. 3A-3B show schematic diagrams of an example exhaled air capture module including a packed bed column, according to some implementations.

FIGS. 3C-3F show perspective views of another exhaled breath collection module, according to some implementations.

FIG. 3G shows a schematic diagram of a method for detecting a LRTI based on capturing proteases in exhaled breath, according to some implementations.

FIG. 4 shows a schematic diagram of an example diagnostic system to detect RTI and other diseases associated with an example exhaled air aerosol collection system, according to some implementations.

FIG. 5 shows a schematic diagram of polymer-peptide conjugate probe including cleavable small molecule fluorophores or chromophores.

FIG. 6 shows the chemical structure of an example substrate-based probe for human neutrophil elastase proteolysis.

FIG. 7A shows a schematic diagram of an example substrate-based probe for examining proteolysis of human neutrophil elastase (“NE”) using MALDI-TOFMS, according to some implementations.

FIG. 7B shows a schematic diagram of another example substrate-based probe for examining proteolysis of human NE proteolysis using MALDI, according to some implementations.

FIG. 7C shows the chemical structure of an example substrate-based probe for examining proteolysis of human NE using MALDI-TOFMS, according to some implementations.

FIG. 7D shows a schematic diagram of four different substrate-based probes associated with a multiplexing assay and their respective characteristic MALDI-TOFMS mass spectra before and after proteolysis of a specific protease in exhaled air, according to some implementations.

FIG. 7E shows the chemical structure of an example substrate-based probe for examining proteolysis of matrix metalloproteinases (MMPs) using MALDI-TOFMS, according to some implementations.

FIG. 7F shows a representative MALDI-TOFMS spectrum of a MMP proteolysis reaction sample solution using a substrate-based probe, according to some implementations.

FIG. 7G shows the chemical structure of another example substrate-based probe for examining proteolysis activity of MMP using MALDI-TOFMS, according to some implementations.

FIG. 7H shows a representative MALDI-TOFMS spectrum of a MMP proteolysis reaction sample solution using another substrate-based probe, according to some implementations.

FIG. 7I shows a representative MALDI-TOFMS spectrum of a solution including three different substrate-based probes for a multiplexing assay associated with exhaled breath analysis, according to some implementations.

FIG. 8A shows a representative MALDI-TOFMS spectrum of a NE proteolysis reaction sample solution.

FIG. 8B shows a representative deconvoluted high-resolution mass spectrum of a NE proteolysis reaction sample solution.

FIG. 9A shows representative MALDI-TOFMS mass spectra of NE proteolysis reaction sample solutions as a function of incubation period during calibration of a NE substrate-based probe, according to some implementations.

FIG. 9B shows a plot of representative ratio of the MALDI-TOFMS mass spectral peak intensity associated with one or more new mass spectra peaks (m/z) to that associated with the one or more characteristic mass spectra peak (m/z) as a function of proteolysis reaction time, according to some implementations.

FIG. 10 shows a plot of the ratio of the MALDI-TOFMS mass spectral peak intensity associated with one or more new mass spectra peaks (m/z) to that associated with the one or more characteristic mass spectra peak (m/z) as a function NE concentration, according to some implementations.

FIG. 11A shows MALDI-TOFMS mass spectra of an example substrate-based probe 700C under NE proteolysis of clinical samples collected from a patient breathing using a ventilator on different days, according to some implementations.

FIG. 11B shows MALDI-TOFMS peak intensity ratio of the peak intensity associated with a new mass spectra peaks (m/z) to that associated with the one or more characteristic mass spectra peak (m/z) as a function of contacting time, according to some implementations.

FIG. 12 shows a plot of MALDI-TOFMS mass spectra of an example substrate-based probe 700C under NE proteolysis of clinical samples collected from a person breathing using a ventilator, according to some implementations.

FIG. 13 shows a schematic diagram of an example method for detecting disease using neutrophil elastase (NE) proteolysis activity of NE captured from aerosolized non-volatile particles in exhaled air, according to some implementations.

FIG. 14A shows MALDI-TOFMS mass spectra of clinical exhaled air samples collected from patients and volunteers after subjecting the samples to human neutrophil elastase (“HNE”) proteolysis using substrate-based probe 700C, according to some implementations.

FIG. 14B shows a box and whisker plot for discriminating between lower respiratory tract infection (“LRTI”) patients and non-LRTI patients and healthy volunteers by determining HNE proteolysis activity of exhaled air samples using a substrate-based probe, according to some implementations.

FIG. 14C shows a receiver operating characteristic (“ROC”) curve with area under the curve (“AUC”) value for discriminating between LRTI and non-LTRI and HV groups by determining HNE proteolysis activity of exhaled air samples using a substrate-based probe, according to some implementations.

FIG. 15A shows a representative ion fragmentation map of a HNE peptide using bottom-up proteomics, according to some implementations.

FIG. 15B shows a protein total intensity profile of proteases and anti-proteases in captured exhaled air samples, according to some implementations.

FIG. 15C shows a correlation plot between HNE concentration and mass spectra intensity of characteristic mass peaks associated with HNE, according to some implementations.

FIG. 16A shows a plot illustrating the specificity of an example substrate-based sensor to proteolysis of rhNE, according to some implementations.

FIG. 16B shows a plot illustrating the sensitivity of an example substrate-based sensor to proteolysis of rhNE, according to some implementations.

FIG. 17A shows box and whisker plots for discriminating between LRTI patients and non-LRTI patients by determining proteolysis activity of exhaled air samples using various substrate-based probes, according to some implementations.

FIG. 17B shows an ROC curve for discriminating between LRTI and non-LTRI by determining protease proteolysis activity of exhaled air samples using a multiplexed assay including three probes, according to some implementations.

FIG. 18 shows a box and whisker plot associated with early detection of LRTI by determining proteolysis activity of exhaled air samples using a HNE proteolysis substrate-based probe, according to some implementations.

FIG. 19 shows a box and whisker plot associated with detecting sepsis cases by determining proteolysis activity of exhaled air samples using a HNE proteolysis substrate-based probe.

Like reference numbers and designations in the various drawings indicate like elements.

### DETAILED DESCRIPTION

The following description is directed to some example implementations for the purpose of describing the innovative aspects of this disclosure. However, a person having ordinary skill in the art will readily recognize that the teachings herein can be applied in a multitude of different ways. The described implementations can be implemented for a variety of applications and may be tailored to compensate for various performance-related deficiencies. As such, the disclosed implementations are not to be limited by the examples provided herein, but rather encompass all implementations contemplated by the attached claims. Additionally, well-known elements of the disclosure will not be described in detail or will be omitted so as not to obscure the relevant details of the disclosure.

Various aspects of the novel systems and methods are described more fully herein with reference to the accompanying drawings. These aspects are provided so that this disclosure will be thorough and complete, and will fully convey the scope of the disclosure to those skilled in the art. Although some examples and aspects are described herein, many variations and permutations of these examples fall within the scope of the disclosure. Although some benefits and advantages of the various aspects are mentioned, the scope of the disclosure is not intended to be limited to benefits, uses, or objectives. The detailed description and drawings are merely illustrative of the disclosure rather than limiting, the scope of the disclosure being defined by the appended claims and equivalents thereof.

In this disclosure, aerosol generally means a suspension of particles dispersed in air or gas. “Autonomous” diagnostic systems and methods mean generating a diagnostic test result “with no or minimal intervention by a medical professional.” In some regions of the world with high burdens of TB infection, access to medically trained personnel is very limited. An autonomous diagnostic system is preferred to one that is not autonomous. The U.S. FDA classifies medical devices based on the risks associated with the device and by evaluating the amount of regulation that provides a reasonable assurance of the device's safety and effectiveness. Devices are classified into one of three regulatory classes: class I, class II, or class III. Class I includes devices with the lowest risk and Class III includes those with the greatest risk. All classes of devices as subject to General Controls. General Controls are the baseline requirements of the Food, Drug and Cosmetic (FD&C) Act that apply to all medical devices. In vitro diagnostic products are those reagents, instruments, and systems intended for use in diagnosis of disease or other conditions, including a determination of the state of health, to cure, mitigate, treat, or prevent disease or its sequelae. Such products are intended for collecting, preparing, and examining specimens taken from the human body. The example devices disclosed herein may operate and produce a high-confidence result and has the potential to be treated as a Class I device.

Breath aerosol particles contain a variety of nonvolatile organic biomolecules such as metabolites, lipids, and proteins including enzymes. The aerosol particles in exhaled breath may also include one or more of microbes, viruses, metabolite biomarkers, lipid biomarkers, or proteomic biomarkers, for example, truncated proteoforms, which are characteristic of respiratory diseases and other diseases. Additionally, these nonvolatile molecules have a wide particle size distribution ranging from a sub-micron size to about 10 microns in size. Breath collection and disease diagnostic systems and methods that can efficiently capture different types of nonvolatile molecules of different particle sizes from exhaled breath are required. Particular aspects of the invention are described below in considerable detail for the purpose of illustrating the compositions and principles, and operations of the disclosed methods and systems. However, various modifications may be made, and the scope of the invention is not limited to the example aspects described.

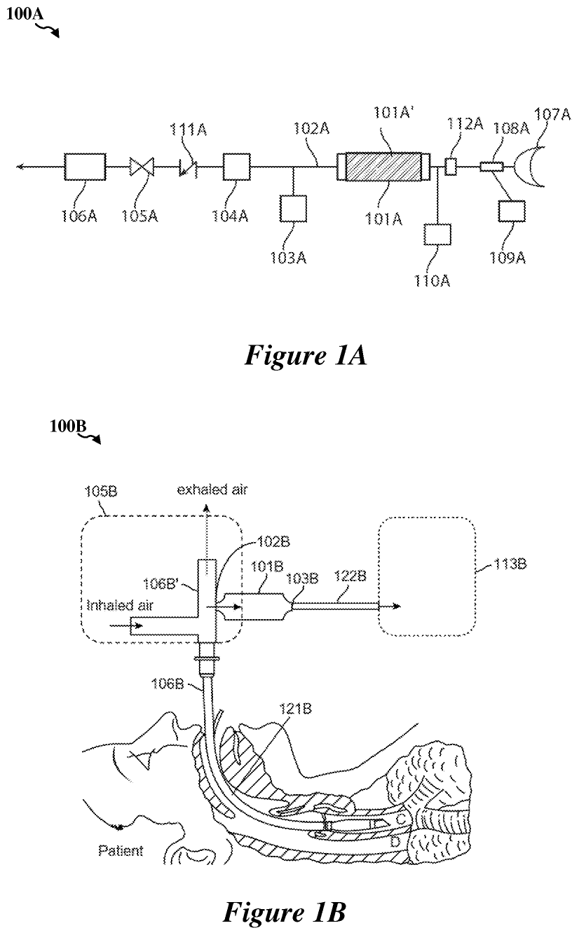

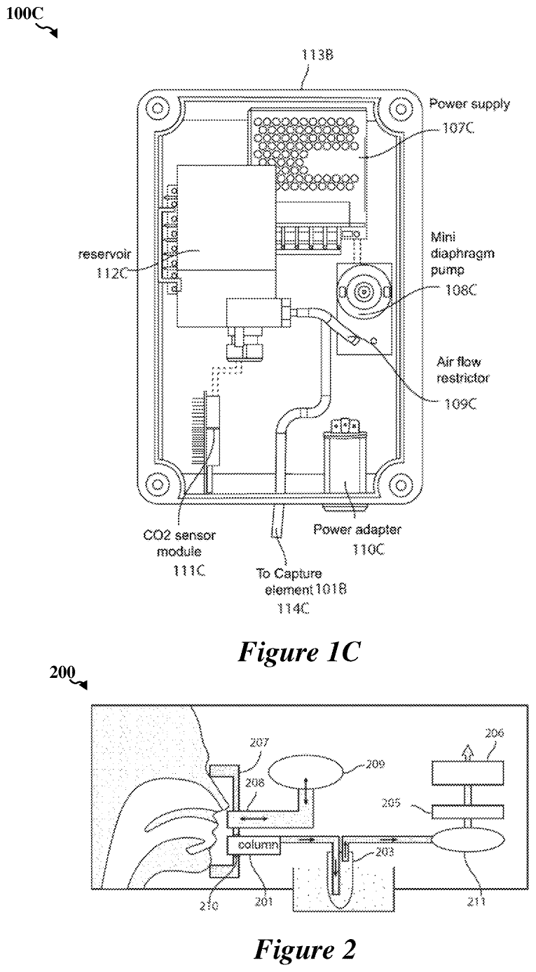

FIG. 1A shows a schematic diagram of an example exhaled breath sample collection system 100A including a packed bed column, according to some implementations. Example exhaled breath sample collection system 100A may include a sample capture element 101A including a packed bed column 101A′ to selectively capture exhaled breath aerosol (which may also be referred to herein as exhaled air aerosol) including nonvolatile organic particles including one or more of bacteria and viruses, and molecules including small molecules, lipids, and proteins including enzymes using high efficiency adsorbent materials. Trap 103A may be disposed in fluid communication with column 101A using tubing 102A. Trap 103A may be made of glass or plastic material. Trap 103A may be cooled to below ambient temperature using an ice bath or other suitable means. Trap 103A may collect water vapor, other volatile and nonvolatile molecules that may pass through the packed bed column 101A′ as exhaled breath condensate (“EBC”).

During breath analysis of a normally breathing person, sample capture element 101A may be removably connected to a mouthpiece (not shown for simplicity) into which the patient is instructed to breathe or otherwise execute a breath maneuver. For example, sample capture element 101A may be removably connected downstream (at the outlet) of a breath collection element 107A including a first aid CPR rescue mask (e.g., as supplied by Dixie USA EMS Supply Co., Model Number EVR-CPR01) worn by the patient during breath analysis. A flow splitter 108A may be disposed between breath collection element 107A and sample capture element 101A to divide the flow of exhaled breath such that a first portion of exhaled breath is directed to capture element 101A and a second portion towards a HEPA filter 109A. Flow splitter 108A may be integrated into breath collection element 107A.

In some implementations, a large-particle trap 112A may be disposed upstream of sample capture element 101A to remove large particles of breath condensate (for e.g., particles greater than about 10 μm) from exhaled breath prior to entering sample capture element 101A. Pump 106A may be used to pull exhaled breath into the packed bed column 101A′ disposed in capture element 101A. An example pump 106A may include a portable diaphragm pump (e.g., Parker Hannifin Corp., Part No.: D737-23-01). The flow rate out of pump 106A may be adjusted using needle valve 105A to achieve a desired flow rate. Check valve (one-way flow valve) 111A may be disposed between pump 106A and sample capture element 101A and may be configured to be in an open position only when pump 106A is pulling exhaled breath through the packed bed column. When there is no flow, valve 111A may be disposed in a closed position. In some implementations, a nominal flow rate of between about 200 ml/min and 600 ml/min may be pulled through packed bed column 101A′ using pump 106A.

To determine if the volume of exhaled breath sample was adequate, a CO₂ sensor and particle counter (not shown for simplicity) may be disposed between breath collection element 107A and sample capture element 101A. Monitoring CO₂ levels and particle count may allow for an approximation of exhaled air volume. A HEPA filter may also be disposed downstream of trap 103A. Capture element 101A may be cooled using a cooling jacket or other means to reduce the temperature to below ambient temperature to increase the collection efficiency of non-volatile organics particles. The breath sample collection system 100A may further include a humidifier 110A disposed upstream of the inlet to the sample capture element 101A to humidify exhaled breath and increase the humidity in the packed bed column.

In some implementations, breath collection element 107A may include a tight-fitting mask configured to receive an individual's face and may be removably attached using straps and the like to the face/head of a patient/individual. The individual may sit in an optional containment booth to isolate the patient's EBA from the ambient air in the testing room or area. Breath collection element 107A may be used to collect and direct breath aerosol particles emitted though the mouth and nose of patient into sample capture element 101A using pump 106A as previously described, without depositing the aerosol particles on the walls of element 107A. Breath collection element 107A may be disposable to limit the risk of a patient becoming contaminated or infected with a pathogen exhaled by a previous patient. In some implementations, breath collection element 107A may be reusable, in which case it may be configured to be sterilized between uses.

In some implementations, the example packed bed column 101A′ in sample capture element 101A may include Hamilton PRP-C18 resin beads, for example, as supplied by Sigma Aldrich and other vendors. The packed bed column 101A′ may be held in place between two porous filter plates such as frit discs. For example, a polyethylene disc having an average pore size of above 35 μm may be placed upstream of the bed (that is, disposed closer to the inlet of sample capture element 101A) and a polyethylene disc having an average pore size of 10 μm (Boca Scientific, Dedham, MA) may be placed downstream of the bed. The 35 μm frit disc minimizes pressure drop across the packed bed column and permits a higher air flow rate while the smaller 10 μm frit disc prevents the loss of C18 resin beads from the packed bed column. In some implementations of an example capture element 101A, the packed bed column may include about 25 mg of C18 resin beads having a nominal diameter between about 12 μm and about 20 μm. Non-volatile organic components in exhaled breath removably interact with the C18 functional groups on the beads and are adsorbed in the packed bed column. Water, volatiles, and other hydrophilic molecules pass through the bed and may be trapped using trap 103A.

In some implementations, in addition to C18 functional groups, other functional groups that show affinity to nonvolatile molecules in EBA may be used as adsorbents in the column. The functional groups may be immobilized on solid phase beads such as resin beads. The solid phase beads may be made of polymers and particles that include one or more of resins, cellulose, silica, agarose, or hydrated Fe₃O₄ nanoparticles. Adsorbent materials may include other functional groups that include one or more of octadecyl, octyl, ethyl, cyclohexyl, phenyl, cyanopropyl, aminopropyl, 2,3-dihydroxypropoxypropyl, trimethyl-aminopropyl, carboxypropyl, benzenesulfonic acid, or propylsulfonic acid disposed on solid phase beads. Functional groups may also include one or more of ion exchange phases, polymer phases, antibodies, glycans, lipids, DNA, or RNA. In some instances, prior to capturing the aerosolized non-volatile particles including one or more proteases, the packed bed column may be activated. In some instances, activating the packed bed column may include one or more of saturating the packed bed column using one or more of organic solvents or water, or by humidifying exhaled air using a humidifier disposed upstream of the sample capture element to increase the humidity in the packed bed column. In some examples, activating the packed bed column may include washing the packed bed column with one or more of isopropyl alcohol or methanol followed by washing with water. Washing the packed bed column with one or more organic solvents may increase the hydrophilicity of the beads in the packed bed column. The sample capture element may be capped at an inlet and an outlet of the sample capture element.

In some implementations, the example sample capture element 101A as described above may also be used to capture aerosolized non-volatile organic particles in exhaled air from patients breathing using a ventilator in an intensive care unit of a hospital. FIG. 1B shows a schematic diagram of an example exhaled air aerosol collection system 100B configured to operate an exhaled air aerosol sample capture system connected to a ventilator, according to some implementations. Ventilator 105B is a life support machine and is used in intensive care units for patients who cannot breathe on their own. For example, persons suspected of being infected with COVID-19 or patients with severe symptoms of COVID-19 or other RTIs may need the assistance of a ventilator to breathe. Second end 121B of Tracheostomy tube 106B (also referred to herein as “exhaled air tubing”) is inserted through the patient's mouth or nose directly into the trachea. The ventilator pushes air into the lungs through tube 106B and forces the person to inhale. The ventilator typically forces air in for one second, pauses for about three seconds to allow the patient to exhale through tube 106B, and then repeats the cycle. Inlet end 102B of sample capture element 101B may be removably connected and preferably directly (that is, without any interconnecting tubing) to first end 106B′ of exhaled air tubing 106B to minimize particle loss. As can be seen, while the outlet end 103B of capture element 101B is removably connected to pump 113B using an interconnecting tubing 122B, there is no such interconnecting tubing between the inlet end 102B of capture element 101B and the first end of exhaled air tubing 106B′. Inlet end 102B may be directly coupled to first end of exhaled air tubing 106B′ using quick connect/disconnect coupling or other suitable fittings or couplings that are known to those skilled in the art.

Sample capture element 101B may be disposed in fluid communication with ventilator 105B. Sample capture element 101B may include a packed bed column (not shown for simplicity) to selectively capture non-volatile particles in exhaled breath aerosol. In some implementations, outlet end 103B of sample capture element 101B may be removably connected to pump 108B in subsystem 113B (as shown with additional details in FIG. 1C) using a tubing to draw in exhaled air through the packed bed column in element 101B at a flow rate of between about 200 ml/min and about 2.5 L/min. In some instances, an example particle capture efficiency associated with exhaled breath aerosol particle capture system may be at least 99%. In some other instances, an example particle capture efficiency associated with exhaled breath aerosol particle capture system may be at least 90%. In some instances, sample capture element 101B may be removably connected to the capnography port on the exhaled air tubing of a ventilator, placing it very near the outlet or at the outlet from the patient's lungs. In some implementations, the sample capture element 101B may be disposed “in-line” with respect to the exhaled air tubing 106B and need not be connected to a bypass branch connected to first end 106B′ of exhaled air tubing 106B. In some instances, exhaled air may be vented out after passing through a HEPA filter (not shown for convenience). That is, exhaled air is not recirculated back to the ventilator. A ventilator with a recirculation loop requires careful balancing of recirculated air. Additionally, a person skilled in the art would recognize that “breaking the circuit” by introducing a recirculation loop connected to first end 106B′ of exhaled air tubing would increase the risk of introducing infection in patients breathing through a ventilator, who are susceptible to infections.

As previously described herein, in some instances, prior to capturing the aerosolized non-volatile particles including one or more proteases, the packed bed column may be activated. In some instances, activating the packed bed column may include one or more of saturating the packed bed column using one or more of organic solvents or water, or by humidifying exhaled air using a humidifier disposed upstream of the sample capture element to increase the humidity in the packed bed column. In some examples, activating the packed bed column may include washing the packed bed column with one or more of isopropyl alcohol or methanol followed by washing with water. Washing the packed bed column with one or more organic solvents may increase the hydrophilicity of the beads in the packed bed column. The sample capture element may be capped at an inlet and an outlet of the sample capture element.

Without being bound by any particular theory, a particle capture efficiency associated with exhaled air aerosol particle capture system of at least 99% may be realized by directly (that is, without any interconnecting tubing) connecting or coupling the sample capture element to the first end 106B′ of exhaled air tubing of the exhaled air tubing 106B to minimize particle loss and activating the packed bed column prior to use, as previously described herein.

In some implementations, an example packed bed column in sample capture element 101B may be activated by saturating with water prior to sample capture. In some examples, an example packed bed column may be kept in a moist or wet state prior to use. In some instances, an example packed bed column may be washed once with 70% ACN and thrice with 0.05% TFA. In some instances, both ends of the example packed bed column may be preferably capped and stored at about 4° C. to avoid freezing. In some other instances, an example packed bed column may be capped at both ends and stored in a refrigerator to prevent drying out of the beads prior to use.

In some implementations, example exhaled air aerosol collection system 100B may include a trap (not shown for simplicity) disposed between sample capture element outlet end 103B and subsystem 113B to collect any condensate in exhaled air. The trap may be cooled to a temperature below ambient temperature. An optional HEPA filter and a needle valve or flow meter (not shown for simplicity) may be installed between the trap and the pump. CO₂ in exhaled breath passes through the packed bed column. To determine if exhaled breath sample volume is adequate, a CO₂ sensor (not shown for simplicity) may be disposed between the sample capture element outlet end 103B and the trap. CO₂ monitoring allows for an approximation of the exhaled air volume. A particle counter (not shown for simplicity) may be installed upstream of sample capture element 101B and also between outlet end 103B and the trap to detect the size and number for particles exiting the packed bed column, which may also be used to detect saturation of the bed and breakthrough of nonvolatile organic molecules from the packed bed column.

FIG. 1C shows a schematic diagram 100C of an example subsystem 113B configured to operate an exhaled breath aerosol sample capture system 100B (as previously described herein) connected to a ventilator, according to some implementations. Sample capture element 101B may be disposed to be in fluid communication with system 113B through port 114C, which may include a quick connect/disconnect coupling. A portion of exhaled air drawn through sample capture element 101B using pump 108C may be routed to reservoir 112C which may be fluidly connected with CO₂ sensor 111C. Reservoir 112C may be a well-sealed container and may be used to prevent any leaks from the CO₂ sensor. Subsystem 113B may include a user interface and an on-off switch to initiate and stop sampling of exhaled breath using sample capture element 101B. Additionally, components such as flow controllers, and flow restrictors 109C may also be packaged in portable subsystem 113B. Subsystem 113B may include a diaphragm pump, such as a mini diaphragm pump 108C. Portable system 113B may be 11 in.×7.5 in.×5.5 in. (L×D×H) and may include noise cancelling materials such as foam pads to reduce the noise level caused by the pump to less than 45 dB. Subsystem 113B may be disposed at a distance from the sample capture element, for example, outside an intensive care unit in a hospital.

In some implementations, as previously described, an example packed bed column disposed in sample capture element 101B may include Hamilton PRP-C18 resin beads, held in place between two porous filter plates or frit discs. In some implementations of sample capture element 101B, the packed bed may include about 25 mg of C18 resin beads having a nominal diameter between about 12 μm and about 20 μm. In some implementations, besides C18 functional groups, other functional groups supported on beads or other particles that show affinity to nonvolatile molecules may be used as adsorbents in the packed bed column. The functional groups may be immobilized on solid phase beads such as resin beads. The solid phase beads may be made of polymers and particles such as resins, cellulose, silica, agarose, and hydrated Fe₃O₄ nanoparticles. Adsorbent materials may include other functional groups that include, one or more of, octadecyl, octyl, ethyl, cyclohexyl, phenyl, cyanopropyl, aminopropyl, 2,3-dihydroxypropoxypropyl, trimethyl-aminopropyl, carboxypropyl, benzenesulfonic acid, and propylsulfonic acid disposed on solid phase beads. Functional groups may also include one or more of ion exchange phases, polymer phases, antibodies, glycans, lipids, DNA and RNA. The packed bed column may be activated prior to use as previously described herein.

In some implementations, for capturing aerosolized virus particles, example sample capture element 101B may include sulfate ester-immobilized cellulose beads. In some implementations, sample capture element 101B may include packed bed columns including C18 beads and sulfate ester-immobilized cellulose beads. In other implementations, sample capture element 101B may include a packed bed column including a mixture of C18 beads and sulfate ester-immobilized cellulose beads. Example sulfate beads may include Cellufine Sulfate beads (JKC Corp., Japan). In some implementations, the particle diameter of the beads may be between about 40 μm and about 130 μm. In some implementations, an example sample capture element 101B may include about 100 mg of sulfate ester-immobilized cellulose beads disposed as a packed bed column. In some implementations, the example sample capture element 101B may have an internal diameter of about 7 mm and length of about 30 mm. The packed bed column may be activated prior to use as previously described herein.

In some implementations, the capacity of the C18 beads in sample capture element 101B configured to capture non-volatile organic molecules may be between about 0.05 mg (non-volatile organics)/mg beads and about 0.5 mg/mg. In other implementations, the capacity of C18-bonded resin beads in the packed bed column in an example sample capture element may be about 0.1 mg/mg. That is, a packed bed column including about 25 mg C18 beads may be characterized by a capacity to capture or adsorb about 2.5 mg of non-volatile organic molecules.

In some implementations, pump 108C may be a diaphragm pump. Data from the CO₂ sensor 111C may be recorded on a non-volatile memory card such as an SD card that is commonly used in portable devices. A flow rate sensor may be installed to monitor the flow rate through the C18 packed bed column. In some implementations, a flow controller may be employed to achieve a consistent flow rate, for example, a flow rate of 500 mL/min through the packed bed column. To enable exhaled air aerosol sampling from a ventilator disposed in hospital intensive care units using example capture element 101B, pump 108C may be packaged along with a CO₂ sensor 111C, associated power supply 107C, system control components, and required fluidic components (for example, tubings, quick connect/disconnect couplings, and the like) in portable system 113B.

FIG. 2 shows a schematic diagram of an example exhaled air aerosol non-volatile particle capture system 200 including a packed bed column, according to some implementations. In some implementations, the example exhaled air capture element 201 may include a packed bed column including C18-bonded resin beads (not shown for simplicity). These resin beads may have C18 functional groups immobilized on the surface. In some implementations, sample capture element 201 may be connected or removably installed to a first aid CPR rescue mask 207 with minor modifications. In some implementations, the stem 208 of mask 207 that usually connects to a resuscitation bag may be modified to removably connect to a HEPA filter 209. The HEPA filter prevents contamination of inhaled breath by contaminants from ambient air. The oxygen inlet 210 to the mask may be located below the stem and may be configured to be proximate to the chin of a human subject when mask 207 is worn by the subject. In some implementations, sample capture element 201 may be removably inserted into mask 207 through inlet or port 210 or otherwise removably connected to or inserted into mask 207 to form a substantially leak-tight fit with mask 207. Port 210 may be disposed below the stem 208 and disposed proximate to the person's chin when the mask 207 is positioned on the person's face. As can be seen, sample capture element 201 may be removably connected directly and without interconnecting tubing to mask 207 to aerosol minimize particle loss. For example, sample capture element may be directly coupled to mask 207 using quick connect/disconnect coupling or other suitable fittings or couplings that are known to those skilled in the art.

Mask 207 may include elastic bands or ties that may be looped behind the head of a human subject to seal the mask to the face of the patient. Mask 207, as described above, prevents direct contact between the mouth and the inlet of the column in element 201, minimizes or eliminates contamination of the column inlet by saliva, and also maximizes non-volatile organic particle collection from exhaled breath.

In some implementations, trap 203 may be immersed in ice water and may be installed downstream of sample capture element 201. The flow rate (air draw rate) using pump 206 may be controlled using needle valve 205 to pull exhaled air at a flow rate of about 600 mL/min. In some implementations, a nominal flow rate of between about 200 ml/min and 600 ml/min may be used. An optional HEPA filter 211 may be installed between trap 203 and needle valve 205. Other fluidic components such as a check valve (for example, as shown in FIG. 1A) may be installed in exhaled breath aerosol non-volatile particle capture system 200 to prevent backflow into the packed bed column disposed in sample capture element 201. CO₂ in exhaled breath passes through the column bed in element 201. To determine if exhaled breath sample volume and/or breathing maneuvers are adequate, a CO₂ sensor (not shown for simplicity) may be disposed between the outlet of breath capture element 201 and trap 203. CO₂ monitoring allows for an approximation of exhaled air volume.

In some implementations, a particle counter (not shown for simplicity) may also be installed between the outlet of element 201 and trap 203 to detect the size and number for particles exiting the packed bed column, which may also be used to detect saturation of the bed and breakthrough of nonvolatile organic molecules from the packed bed column. Example exhaled breath aerosol non-volatile particle capture system 200 may also include a sample capture element 201 bypass line (not shown for simplicity) to enable standardization of breath volume prior to routing into the column bed in element 201. A CO₂ sensor and particle counter may also be fluidly connected to the bypass line. The capacity of solid beads immobilized with functional groups in the packed bed column in capture element 201 may be between about 0.05 mg (non-volatile organics)/mg beads and about 0.5 mg/mg. The capacity of C18-bonded resin beads in the packed bed column disposed in example sample capture element 201 may be about 0.1 mg/mg. That is, a packed bed column having 25 mg C18 beads may have the capacity to trap or adsorb about 2.5 mg of non-volatile organic molecules. The packed bed column may be activated prior to use as previously described herein.

As previously discussed herein, besides C18 functional groups, other functional groups that show affinity to non-volatile molecules may be used as adsorbents in the packed bed column in sample capture element 201. These functional groups may be immobilized on solid phase beads such as resin beads. In some implementations, the solid phase beads may be made of polymers and particles including one or more of resins, cellulose, silica, agarose, or hydrated Fe₃O₄ particles. Adsorbent materials may include other functional groups that include one or more of octadecyl, octyl, ethyl, cyclohexyl, phenyl, cyanopropyl, aminopropyl, 2,3-dihydroxypropoxypropyl, trimethyl-aminopropyl, carboxypropyl, benzenesulfonic acid, or propylsulfonic acid disposed on solid phase beads. Functional groups may also include one or more of ion exchange phases, polymer phases, antibodies, glycans, lipids, DNA, or RNA. The packed bed column may be activated prior to use as previously described herein.

In some implementations, an exhaled breath capture module that minimizes backpressure exhaled air sample collection may be desired. Backpressure may cause extreme discomfort to the person or patient during exhalation of breath for diagnosis of a respiratory disease, in particular, to patients with pulmonary problems. Accordingly, breath collection systems that include baffles or other impediments to the flow of exhaled breath are undesirable and ineffective as they would cause increased backpressure and cause extreme discomfort to the person or patient. Additionally, a capture efficiency of non-volatile organic particles aerosolized in exhaled breath of greater than 99% may be needed. Accordingly, capture of aerosolized particles using a film disposed on a porous support material is ineffective as they would be quickly saturated with moisture, water droplets, and the like in exhaled breath, leading to poor particle capture efficiency. In some implementations, an exhaled breath capture module that minimizes backpressure during capture of non-volatile organic particles aerosolized in exhaled breath over a period of at least 10 minutes, and at least 99% particle capture efficiency is desired.

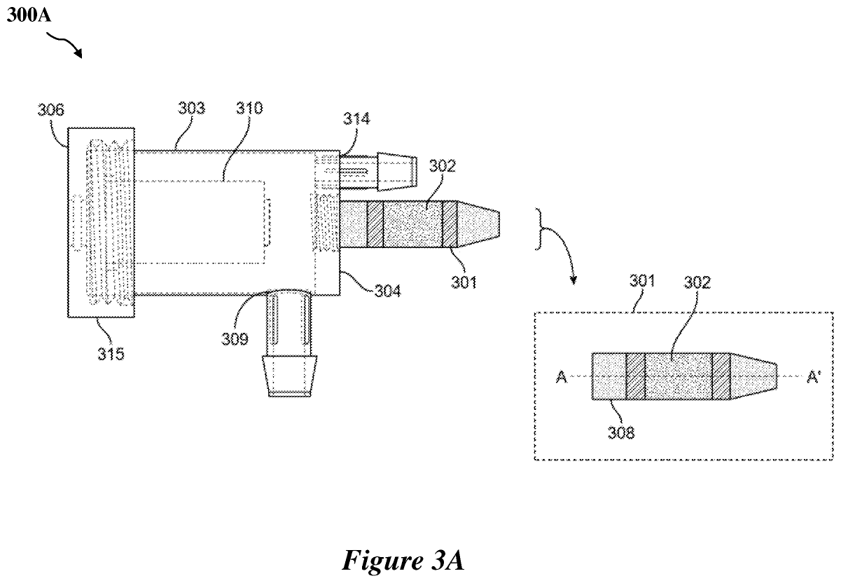

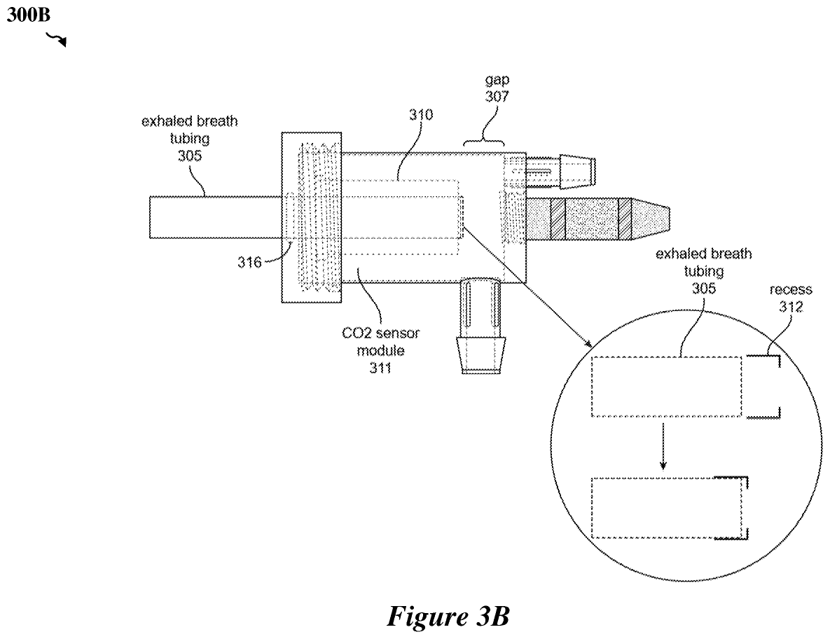

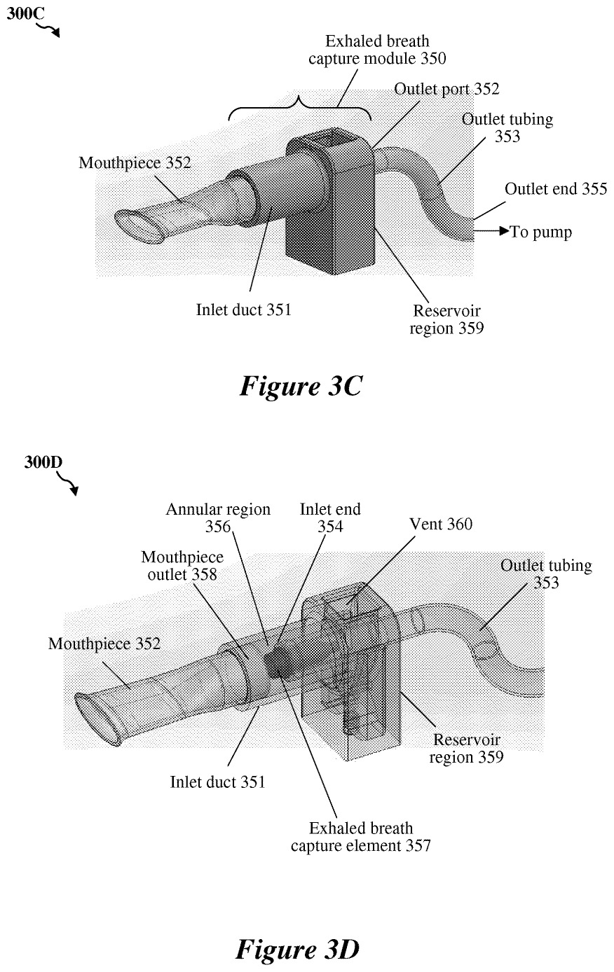

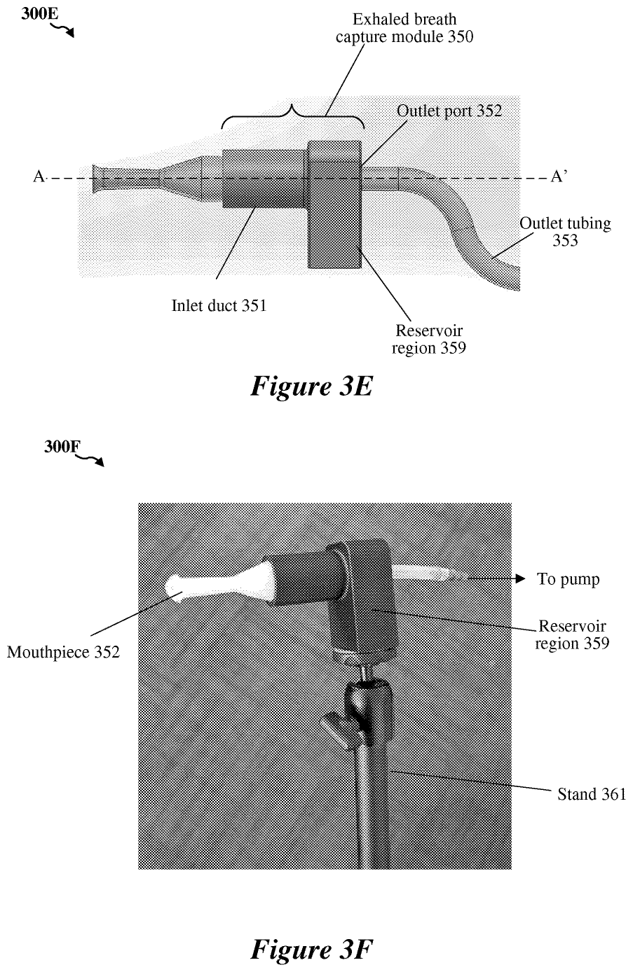

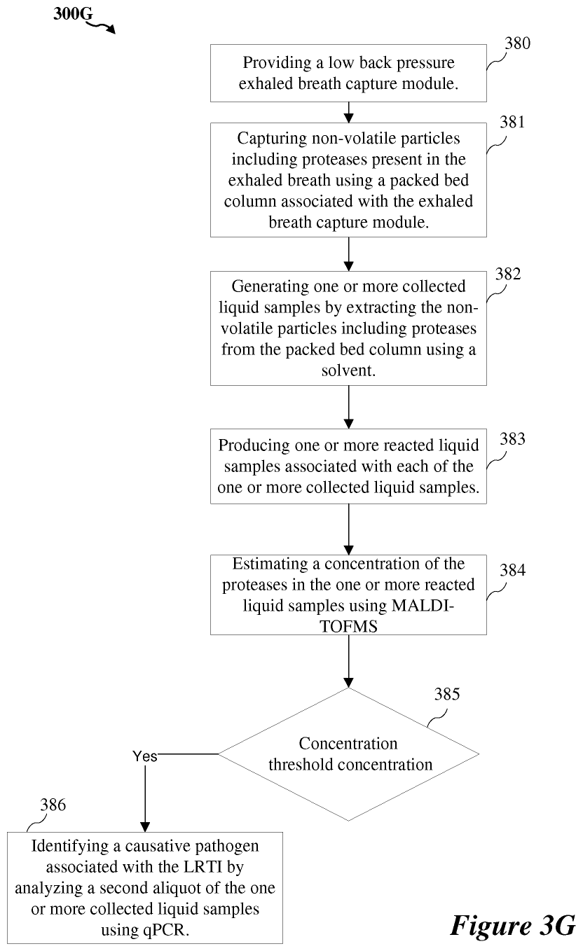

FIGS. 3A-3B show schematic diagrams of an example exhaled breath capture module 300 including a packed bed column, according to some implementations. Example breath capture module 300A-300B may be used to minimize backpressure during the capture of non-volatile organic particles including biomaterials aerosolized in exhaled breath over a period of at least 10 minutes and at least 99% particle capture efficiency. Module 300 may include an exhaled breath capture element 301 including a packed bed column 302 disposed between an inlet end and an outlet end of the breath capture element 301. The exhaled breath capture element 301 may selectively capture non-volatile organic particles in breath exhaled by a person. Module 300 may include an exhaled breath management chamber 303. In some implementations, exhaled breath capture element 301 may be removably connected to chamber outlet end 304. An exhaled breath tubing 305 may be removably inserted through chamber inlet end 306, until a gap 307 of predetermined length is defined between an outlet end of the exhaled breath tubing 305 and the capture element inlet end 308. A pressure relief port 309 may be disposed near chamber outlet end 304, and proximate to the gap 307. In some implementations, chamber 303 may include more than one pressure relief ports. Breath exhaled by a person may be drawn into the chamber through the exhaled air tubing using a pump disposed in fluid communication with the exhaled breath sample capture element 301. In some implementations, breath capture element outlet end 313 may be disposed external to the exhaled breath management chamber. In some other implementation, chamber 303 may be configured to house the exhaled breath sample capture element 301. In some other implementations, sample capture element 301 may be disposed in a suitable enclosure (not shown for simplicity) that abuts outlet end 304 of chamber 303. The pump may draw exhaled breath through the sample capture element at a nominal flow rate of between about 0.5 L/min and about 10 L/min.

In some implementations, exhaled breath management chamber 303 may include an adapter element 310 disposed inside the chamber to define an annular region 311 between the adapter and the chamber. Exhaled air tubing 305 may be removably inserted through a port disposed in the chamber inlet end 306 and through the adapter 310. An O-ring 316 disposed on chamber inlet end 306 may be used to provide a seal between tubing 305 and chamber 303. The adapter 310 may include a recess 312 to stop the extent to which the exhaled air tubing 305 may be inserted or may travel into the adapter 310. Recess 312 may be positioned to define the gap 307 of predetermined length between the outlet end of the tubing and capture element inlet 308. That is, the recess may serve as a back-stop element in the adapter to stop the travel of the exhaled air tubing through the adapter 310 and into the chamber 303. The adapter 310 may be removably disposed inside chamber 310. The inlet end of chamber 306 may include or may be defined by a removable chamber cap 315. The adapter 310 may be removed from chamber 303 by opening the cap.

In some implementations, the non-volatile organic particles in exhaled breath to be captured by the sample capture element 301 may include one or more of metabolite biomarkers, lipid biomarkers, proteomic biomarkers, proteins and enzymes, bacteria particles, or virus particles characteristic of at least one respiratory disease. The example exhaled breath capture element 301 may have a particle capture efficiency of greater than 99%.

In some implementations, the exhaled breath tubing 305 may be made of one or more of paper, plastic or metal. The breath tubing may be rigid, flexible, or substantially rigid. The pressure relief port 309 may be disposed orthogonal to a longitudinal axis (A-A′) of the exhaled breath sample capture element 301 when the capture element is removably connected to the chamber outlet end 304. In some implementations, the exhaled breath capture module 300 may further include one or more instrument ports 314 disposed at the chamber outlet end 304. The instrument port 314 may be disposed in fluid communication with one or more of a particle counter or a CO₂ sensor (as described below).

In some implementations, the exhaled breath capture element inlet end 308 may have nominal diameter substantially equal to the diameter of the exhaled breath tubing. The example packed bed column 302 may include solid particles of one or more of resins, cellulose, silica, agarose, or hydrated Fe₃O₄ particles. In some implementations, the packed bed column may include one or more of resin beads having C18 functional groups on the surface, or cellulose beads having sulfate ester functional groups on the surface. In some implementations, the resin beads or cellulose beads may have an average diameter of between about 10 μm and about 10 mm. The resin beads or cellulose beads may be packed between two porous polymeric frit discs. Example porous frit discs may include polyethylene discs supplied by Boca Scientific (Dedham, MA). Additional details are disclosed in commonly-owned U.S. Prov. Pat. Appl. No. 63/469,307, which is incorporated by reference herein in its entirety.

In some implementations, the beads in packed bed column 302 may be functionalized with one or more functional groups immobilized on the surface of the particles. The functional groups may include one or more of C18 (octadecyl), octyl, ethyl, cyclohexyl, phenyl, cyanopropyl, aminopropyl, 2,3-dihydroxypropoxypropyl, trimethyl-aminopropyl, carboxypropyl, benzenesulfonic acid, propylsulfonic acid, an ion exchange phase, a polymer phase, antibodies, glycans, lipids, DNA, or RNA. The ion exchange phase may include one or more of diethylaminoethyl cellulose, QAE Sephadex, Q sepharose, or carboxymethyl cellulose. The polymer phase may include one or more of polystyrene-co-1,4-divinylbenzene, methacrylates, polyvinyl alcohol, starch, or agarose. The antibodies may include one or more of anti-human albumin, anti-Influenza A virus nucleoprotein (“NP”), or Anti-SARS-CoV-2 virus NP. The packed bed column may be activated prior to use as previously described herein.

In some implementations, the packed bed column length (L) in sample capture element 101A, 101B, 201, or 301 may be about 3 mm. The nominal internal diameter (D) of the tube may be about 7 mm. An example packed bed including about 25 mg of C18 resin beads having a nominal particle diameter (Dₚ) of between about 12 μm and 20 μm, yields a L/Dₚ ratio of between about 150 and 250 at a D/Dₚ ratio of about 350 to about 580. These column parameters may prevent undesirable localized flow distributions in the bed to ensure that substantially all resin beads were exposed to the aerosol flow through the bed.

In some implementations, non-intubated patients diagnosed with a respiratory tract infection may find it difficult to provide an exhaled breath sample for diagnosis and treatment. In some instances, these patients may inhale supplemental oxygen supplied via a cannula inserted into the patient's nostrils to alleviate difficulties with breathing. A nasal cannula is a flexible tube inserted into a patient's nostrils and is coupled to an oxygen source. A nasal cannula may be used to reduce difficulties in breathing by supplying oxygen at rates ranging from 1 liter/min to 6 liter/min. A patient may inhale the supplemental oxygen while exhaling into a breath capture device. Accordingly, a mask-based exhaled breath capture device as previously described herein with reference to FIG. 2 may not be suitable for collecting exhaled breath samples from patients who are inhaling supplemental oxygen supplied via a cannula because routing a cannula through a mask is not practical. There is a need for exhaled breath capture devices that reduce or eliminate the discomfort of patients breathing via a cannula during the collection of exhaled breath samples by minimizing back pressure generated by a breath capture device on a patient's breathing. Exhaled breath may be collected from the patient exhaling through the patient's mouth into a mouthpiece. An example of one exhaled air capture module was previously described herein with reference to FIGS. 3A-3B.

FIGS. 3C-3F show perspective views 300C-300F of another exhaled breath collection module 350, according to some implementations. In various other implementations, exhaled breath capture module 350 configured to capture aerosolized non-volatile organic particles in exhaled breath may include an inlet duct 351 configured to receive a mouthpiece 352 removably insertable into the inlet duct 351, and an outlet port 352 disposed opposite to the inlet duct. An outlet tubing 353 including an inlet end 354 and an outlet end 355 may be removably insertable into the outlet port 352 and extend into the inlet duct 351 defining an annular region 356 between the outlet tubing 353 and the inlet duct 351. In some instances, outlet tubing 353 may be configured to include one or more couplings and tubing sections. Those skilled in the art would recognize that the outlet tubing 353 shown as a single tubing section in FIG. 3D is shown by way of example only and may be modified to include one or more tubing sections coupled with suitable couplings without departing from the scope and spirit of the present implementations.

An exhaled breath capture element 357 including a packed bed column configured to capture the aerosolized non-volatile organic particles in exhaled breath may be removably insertable or coupled into the inlet end 354 of the outlet tubing 353 defining a gap of predetermined length (not shown for simplicity) between an outlet 358 of the mouthpiece 352 and the exhaled breath capture element 357.

In some instances, the exhaled breath capture module 350 may include a reservoir region 359 disposed in fluid communication with the inlet duct 351 and the annular region 356 and may be configured to collect saliva particles or breath condensate particles in the exhaled breath that drop through the gap.

In some instances, the exhaled breath capture module 350 may include a vent 360 disposed in fluid communication with the reservoir region 359. Vent 360 may be configured to prevent pressure build-up in the exhaled breath capture module 350 and to subsequently prevent discomfort when a patient who is experiencing a difficulty in breathing is asked to provide an exhaled breath sample by a medical professional. In some other instances, the exhaled breath capture module 350 may further include a HEPA filter (not shown for simplicity) coupled to the vent 360. In some implementations, the vent 360 may be disposed in an orthogonal orientation relative to a longitudinal axis (A-A′) associated with the inlet duct 351.

In some implementations, the outlet end 355 of outlet tubing 353 may be coupled to a pump (not shown for simplicity) configured to draw the exhaled breath through the packed bed column housed in the exhaled breath capture element 357. In some examples, while a first part of the exhaled breath is drawn into the exhaled breath capture element 357 by the pump, a second part of the exhaled breath may bypass the exhaled breath capture element 357, pass through the annular region 356 and exit module 350 through the vent 360. Accordingly, minimal to low back pressure is exerted by the exhaled breath capture element 357 on the patient inhaling oxygen supplied by a cannula, and the patient may provide the exhaled breath sample with minimal or no discomfort. In some instances, a patient breathing into the breath capture module 350 may hold on to the reservoir region 359. In some other instances, and referring to FIG. 3F, a bottom end of the reservoir region 359 may be detachably coupled to stand 361. The stand 361 may be adjusted in height for further eliminating any discomfort to a patient breathing into the exhaled breath capture module.

In some implementations, the packed bed column housed in exhaled breath capture element 357 may include solid particles including one or more of resins, cellulose, silica, agarose, or hydrated Fe₃O₄ particles. The solid particles may be disposed between porous polymeric frit discs. In some instances, the packed bed column may include resin beads having one or more of C18 functional groups on the surface, cellulose beads having sulfate ester functional groups on the surface, or a mixture thereof. In some other instances, the resin beads may have a diameter of at least about 20 μm.

In some implementations, the packed bed column housed in exhaled breath capture element 357 may be activated by one or more of washing the packed bed column with one or more of isopropyl alcohol or methanol, or by washing with water. In some examples, after activating the packed bed column, each of the inlet and the outlet of the exhaled breath capture element 357 may be capped to prevent drying out of the packed bed column. In some instances, the aerosolized non-volatile organic particles in the exhaled breath may include one or more of metabolite biomarkers, lipid biomarkers, proteomic biomarkers, bacteria particles, or virus particles characteristic of one or more respiratory diseases.

In some implementations, the packed bed column housed in exhaled breath capture element 357 may include a first packed bed including silica gel beads and a second packed bed including resin beads functionalized with C18 groups. The second packed bed may be disposed downstream of the first packed bed. In some instances, the silica gel beads may have an average diameter of between about 20 μm and about 500 μm. In some other instances, the resins beads functionalized with C18 groups may have an average diameter of between about 20 μm and about 500 μm.

In some implementations, the pump may be configured to draw exhaled breath through the exhaled breath capture element 357 at a flow rate of between about 0.5 L/min and about 10 L/min.

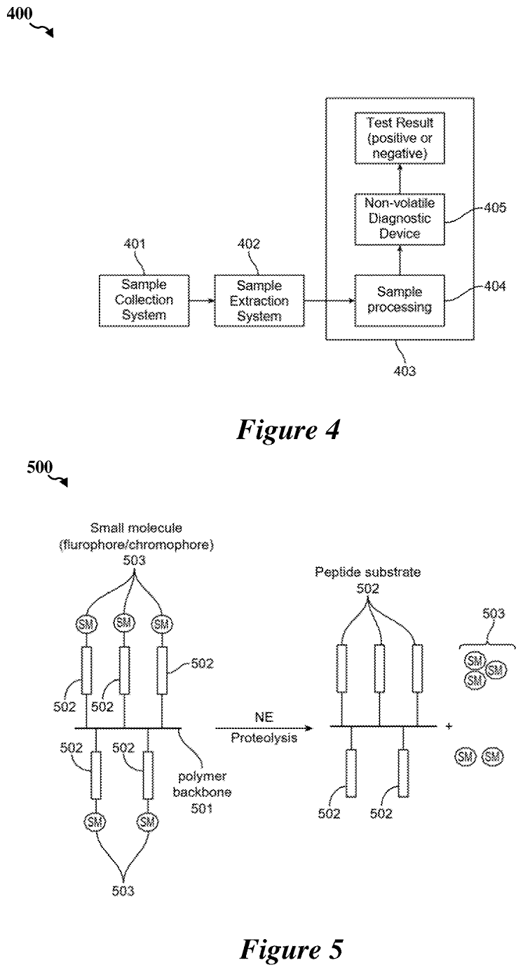

In some other implementations, a system, for example system 400 previously described herein with reference to FIG. 4, for diagnosing a respiratory disease may include the exhaled breath capture module 350 as previously described herein, an extraction means 402 configured to produce one or more liquid samples by extracting the aerosolized non-volatile organic particles from the packed bed column using a solvent. In some instances, the solvent may include one or more of acetonitrile (“ACN”), methanol, trifluoro acetic acid (“TFA”), or isopropanol (“IPA”), the remaining being water. In some examples, the solvent may include between about 50 vol.-% and about 70 vol.-% acetonitrile in water, between about 50 vol.-% and about 70 vol.-% isopropanol in water, and between about 0.05 vol.-% TFA in water.

In some implementations, system 400 mat include an analytical device 405 configured to analyze the particles extracted into the one or more liquid samples. In some instances, the analytical device 405 may include one or more of a polymerase chain reaction (“PCR”) device, Enzyme-Linked Immunosorbent Assay (“ELISA”), reverse transcription PCR (“rt-PCR”), mass spectrometer (“MS”), matrix assisted laser desorption ionization based mass spectrometer (“MALDI-MS”), electrospray ionization mass spectrometer (“ESI-MS”), MALDI time-of-flight mass spectrometer (“MALDI-TOFMS”), or liquid chromatography tandem mass spectrometer (“LC-MS/MS”).

FIG. 3G shows a schematic diagram of a method 300G for detecting a lower respiratory tract infection (LRTI) based on capturing proteases in exhaled breath, according to some implementations. In some implementations, method 300G may begin at 380 with providing a low-back pressure exhaled breath capture module. An example of a low-back pressure exhaled breath capture module was previously described herein with reference to FIGS. 3C-3F. In some instances, method 300G may continue at 381 with capturing one or more proteases (non-volatile organic particles) present in the exhaled breath using the packed bed column. Examples of packed bed columns were previously described herein, and as such, the description of all like elements is not repeated herein.

In some examples, the exhaled breath may be collected from a patient inhaling supplemental oxygen supplied via a cannula inserted into the patient's nostrils, allowing the patient to inhale the supplemental oxygen while exhaling into the mouthpiece associated with the exhaled breath capture module. The exhaled breath may be collected from a patient using an exhaled breath capture module previously described herein with reference to FIGS. 3C-3F. In some other instances, the packed bed column may be activated prior to capturing the non-volatile particles including proteases.

At 382, operation 300G may continue with generating one or more collected liquid samples by extracting the non-volatile particles including proteases from the packed bed column using a solvent. In some implementations, the solvent associated with the extracting operation 382 may include one or more of acetonitrile (“ACN”), methanol, trifluoro acetic acid (“TFA”), or isopropanol (“IPA”), the remaining being water. In some instances, the solvent may include between about 50 vol.-% and about 70 vol.-% acetonitrile in water, between about 50 vol.-% and about 70 vol.-% isopropanol in water, and between about 0.05 vol.-% TFA in water.

In some implementations, operation 300G may continue at 383 with producing one or more reacted liquid samples associated with each of the one or more collected liquid samples by contacting a first aliquot of the one or more collected liquid samples with a plurality of substrate-based probes to disengage a cleavable molecule associated with each of the substrate-based probes in the plurality of substrate-based probes in response to proteolysis activity associated with the interaction between each of the substrate-based probes and the proteases. In some implementations, the plurality of substrate-based probes may include PEG36-Nle(O-Bzl)-Met(O)2-Oic-Abu-ACC, PEG20-PEG20-PLGLKARR, or PEG25-PEG20-KPLGLKAR. In some instances, the proteases may include one or more of human neutrophil elastase, trypsinogen, serine protease 22, Kallikreins, or matrix metalloproteinases (“MMPs”). Additional details are related to the substrate-based probes associated with proteolysis activity are described below in this disclosure.

In some implementations, operation 300G may continue at 384 with estimating a concentration of the proteases in the one or more reacted liquid samples by analyzing the one or more reacted liquid samples using MALDI-TOFMS. Additional details related to processing of mass spectral data associated with proteolysis activity are described below in this disclosure.

In some other implementations, operation 300G may continue at 385 with examining if the concentration of the one or more proteases in the one or more reacted liquid samples is equal to or greater than a cut-off threshold concentration corresponding to the proteases. In some instances, the cut-off threshold concentration is associated with a lower respiratory tract infection (“LRTI”). In some instances, the cut-off threshold concentration associated with the proteases may be between about 0.15 picomol (pM) and about 0.5 pM. If the concentration of the one or more proteases in the one or more reacted liquid samples is equal to or greater than a cut-off threshold concentration, operation 300G may continue at 386 with identifying a causative pathogen associated with the LRTI by analyzing a second aliquot of the one or more collected liquid samples using quantitative polymerase chain reaction (“qPCR”).

FIG. 4 shows a schematic diagram of an example diagnostic system 400 to detect RTI and other diseases including an example exhaled air aerosol collection system and sample processing system and a diagnostic device, according to some implementations. Example diagnosis system 400 may include an exhaled air collection system 40, a sample extraction system 402, and an analysis system 403. The sample collection system 401 may include any of the sample capture elements (e.g., 101A, 101n, 201 or 301) previously described herein. After a predetermined sample collection period, the sample capture element may be removed from system sample collection system 401.

In some implementations, the sample capture element may be autoclaved at 110° C. for about 10 minutes to disinfect the sample capture element prior to extracting the captured aerosol particles. In some implementations, captured non-volatile aerosol particles may be extracted from the packed bed column in the sample capture element using extraction system 402 by washing (or flushing) the packed bed column with about 200 μL to about 400 μL of one or more organic solvents including one or more of about 50% to about 70% acetonitrile (“ACN”), about 50% to about 70% methanol, or about 50% to about 70% isopropyl alcohol (“IPA”). In some implementations, a 50% flush may be used to elute or extract metabolites and proteins (including enzymes) in a first-stage flush, followed by 70% IPA flush to elute lipids from the packed bed column. In some implementations, the organic solvent may be removed, if needed, from the packed bed column by lyophilization overnight to preserve the captured non-volatile bioaerosol particles. In some implementations, the organic solvent may be also removed by incubating on a heating block at about 70° C. for about 30 minutes. Finally, in some implementations, the bed may be washed with about 0.05% trifluroacetic acid (“TFA”).

In some implementations, sample extraction system 402 may be disposed in-line or off-line to sample collection system 401. When extraction system 402 is disposed off-line, at the conclusion of exhaled breath sample collection, the sample capture element may be removed from system 401 and eluted with one or more organic solvents in extraction system 402 to remove non-volatile organics from the packed bed column, as previously described. In some implementations, an example organic solvent may include about 50% to about 70% acetonitrile in water to extract trapped non-volatile organics (e.g., strongly polar non-volatile organic molecules, proteins including enzymes, and the like) from the packed bed column. In some implementations, extraction may be repeated using the same solvent or another solvent. In some implementations, a solvent including about 50% to about 70% isopropanol in water may be used to extract less polar lipid molecules from the packed bed column. In some other implementations, organic solvents including between about 50% and about 70% methanol in water, or about 50% methanol in about 50% chloroform may be used in sample extraction system 402.

In some implementations, sample extraction system 402 may be disposed in-line in system 400. When system 402 is disposed in-line, at least one of a CO₂ sensor and particle counter may be disposed upstream of extraction system 402. In some implementations, extraction system 402 may include a solvent vessel, a pump to transfer the solvent from the solvent to packed bed column, and a vessel to collect the solvent comprising the non-volatile biomarkers into another vessel or cup. In some implementations, system 402 may include an injector to inject any one of the organic solvents previously described herein into the packed bed column and collect the extracted liquid including non-volatile organics and biomarkers in a suitable cup or vessel, or other laboratory tubes having a small volume. The captured sample in solvent may be further processed and analyzed in analysis system 403.

Analysis system 403 may include sample processing system 404 and at least one diagnostic device 405. In some implementations, more than one diagnostic device 405 may be adapted for use in analysis system 403 that include, but are not limited to, devices that perform genomics-based assays (for example, PCR, rt-PCR, and whole genome sequencing), biomarker recognition assays (for example, ELISA), and spectral analysis including mass spectrometry (“MS”). Of these diagnostic devices, MS may be preferable due to its speed of analysis. The MS techniques that may be used for biomarker identification include electrospray ionization (“ESI”) and matrix assisted laser desorption ionization (“MALDI”) time of flight MS (“TOFMS”). ESI may be coupled to high resolution mass spectrometers. MALDI-TOFMS devices may be compact, lightweight, consume less than 100 watts of power and provide sample analysis in less than 15 minutes. MALDI-TOFMS may be used as a diagnostic device for point-of-care diagnostics suitable for ACF.

In MALDI-TOFMS analysis, the sample may be coated with a MALDI matrix and dried in sample processing step 404 before it is inserted into the vacuum chamber of the MS. The sample, often disposed on a sample plate, is subjected to one or more laser pulses from an ultraviolet laser or an infrared laser to create large, informative biological ion clusters that are characteristic of the biological material. When a concentrated sample is provided by sample processing system 404 that include only trace levels of water or trace levels organic solvents such as 50% to 70% of one of acetonitrile, methanol, and isopropanol in water, sample analysis using MALDI-TOFMS may take less than 5 minutes (including sample preparation) because less time is needed to evaporate or remove water and solvents from the sample.

In MALDI-MS, the target sample particle (analyte) is coated with a matrix chemical, which preferentially absorbs light (often ultraviolet wavelengths) from a laser. In the absence of the matrix, the biological molecules would decompose by pyrolysis when exposed to a laser beam in a mass spectrometer. The matrix chemical also transfers charge to the vaporized molecules, creating ions that are then accelerated down a flight tube by the electric field. For MALDI-MS analysis, a liquid, usually including an acid, such as trifluoroacetic acid (“TFA”), and a MALDI matrix chemical including alpha-cyano-4-hydroxycinnamic acid, is dissolved in a solvent and added to the sample. Solvents include acetonitrile, water, ethanol, and acetone. TFA is normally added to suppress the influence of salt impurities on the mass spectrum of the sample. Water enables hydrophilic proteins to dissolve, and acetonitrile enables the hydrophobic proteins to dissolve. The MALDI matrix solution is spotted on to the sample on a MALDI plate to yield a uniform homogenous layer of MALDI matrix material on the sample. The solvents vaporize, leaving behind only the recrystallized matrix with the sample spread through the matrix crystals. When the sample includes live cells, the acid partially degrades the cell membrane of the sample making the proteins available for ionization and analysis in an MS. Other MALDI matrix materials include 3,5-dimethoxy-4-hydroxycinnamic acid (sinapinic acid), α-cyano-4-hydroxycinnamic acid (α-cyano or α-matrix) and 2,5-dihydroxybenzoic acid (DHB) as described in U.S. Pat. No. 8,409,870.

Device 405 may implement one or more analytical methods for analyzing metabolites, proteins, and lipids and may include silver staining for protein profiling, protein assay for protein content, bottom-up proteomics, LC-MS/MS for metabolomics and lipid-omics, and MALDI-TOF mass spectrometry for molecule profiling.

In some implementations, sample processing system 404 may include the necessary components to implement methods including one or more of the following steps:
(a) Placing the sample in at least one of a cup, a vial and a sample plate. For example, the Series 110A Spot Sampler (Aerosol Devices) may be used. The Series 110A Spot Sampler includes 32 well plates with circular well shape (75 μL well volume) or teardrop well shape (120 μL well volume) which are heated to evaporate the solvent and excess fluid/liquid in the sample to concentrate the sample;
(b) Placing the sample in a cup and exposed to a vacuum source or freeze-drying device to cause the solvent to evaporate to concentrate the sample;
(c) hot digestion of proteins and virus particles.

In some implementations, the samples may be centrifuged to remove chemical contamination particles.

Virus (e.g., SARS-CoV-2) detection is centered on detection of viral proteins, which is a difficult challenge. An example method for virus detection may include glycan-based capture matrix (beads) to pull the target virus out of a background matrix (e.g., other non-virus biomolecules, contaminants). Example sample capture element 101A, 101B, 201, or 301 may include glycan-based capture beads packed in a packed bed column. An aliquot of the sample collected using sample capture element 101A, 101n, 201 or 301 may include other background contaminants and may be applied to a bead carrying the capture probe. At least one of glycan, heparin, and carbohydrates may be used as capture materials or probes bound on resin beads or similar types of beads in sample capture element 101A, 101B, 201 or 301.

In some implementations, an optional washing step may be used to remove any nontargeted-virus contaminants. The concentrated and purified virus may be eluted off the beads using one of the solvents previously described herein into a sealed heating chamber containing an organic acid which may include formic acid or acetic acid and heated to 120° C. for about 10 minutes to digest the proteinaceous toxin down into specific peptide fragments. This hot acid protein digestion protocol cleaves the protein at aspartic acid residues creating a highly reproducible peptide pattern. The capture and digestion processes described may be accomplished with antibodies and enzymes, respectively. Using this example sample processing for MALDI-TOFMS, sensitivity for ricin biotoxin of better than 100 ng/mL (with S/N of about 50:1) in clean buffer may be achieved. At S/N (signal to noise ratio) of 3:1, limits of detection (“LOD”) of <10 ng/mL may be achieved. For the 1 μL samples used in the MALDI-TOFMS analytical systems, about 10 ng/mL LOD equates to a total mass of about 10 pg (10⁻¹² g) on the probe, which is equivalent to about 20,000 viral particles.

In some implementations, an example microfluidic sample processing system to implement any one of the methods disclosed above may be configured to analyze samples collected from the air or from other sources such as nasal swabs. The glycan-based sample capture element and other microfluidics components may be reusable. Large fluid reservoirs containing buffer, weak acids, and alcohols may be employed to provide sufficient capacity to measure 100's of samples in one channel of the system. Multiple systems may be run in parallel to process multiple samples simultaneously. Since no fragile and expensive biomolecular reagents are required, the system and methods for analysis of captured aerosolized non-volatile biomaterial in exhaled air is cost effective.

Hot acid digestion cleaves proteins reproducibly at aspartic acid residues creating known peptide sequences with known masses. These peptide mass distributions are characteristic of the progenitor proteins. Thus, hot digestion provides outstanding specificity if the proteins of interest are largely separated from background materials. Furthermore, the peptide mass distribution is directly determined by the genome, accounting for post-translational modifications. As soon as a new virus is isolated, it is rapidly sequenced. The RNA sequence of the SARS-CoV-2 virus may be used to accurately predict the protein sequences with modern bioinformatics tools (ExPASy bioinformatics portal). These proteins may then be “digested” in silico using bioinformatics tools to create a theoretical peptide map. Thus, the peptides that arise from SARS-COV-2 digestion can be predicted and compared to experimental data to generate a specific MALDI TOFMS signature of the organism. Reports suggest that the predominant proteins in SARS-CoV are characterized by about 46 kDa nucleocapsid protein and the 139 kDa spike proteins. Other proteins in reasonable abundance are E, M and N proteins.

Detection specificity of a target virus may require some level of background removal from MS spectra, particularly if the background contains other proteins. If large amounts of exogenous proteins are present, the peptide map could be dominated by non-target peptides. As previously described, affinity capture probes for the virus toxins based on glycan-decorated agarose beads may be used to readily clean up the toxins, even in large excess of background proteins, and other biomolecules. When analyzing exhaled breath for virus targets such as SARS-CoV-2, other human proteins in breath may interfere with detection specificity. An affinity-based cleanup of the sample is required to ensure good specificity. Virus detection may require bead materials that provide more selective affinity compared to the glycan-decorated beads previously described.

In some implementations, dextran-based adsorbents may be used for purifying viruses, including coronaviruses. In some implementations, carbohydrates may be used for viral and protein purification including target viruses such as SARS-CoV and SARS-CoV-2. Further heparin, and heparan sulfate may be used as binding agents bound to resin beads. Heparin covalently linked to agarose beads (GE Healthcare Life Sciences, Heparin Sepharose 6 Fast Flow affinity resin Product #17099801) may be used instead of glycan capture beads. This resin may enable bead-based capture affinity capture system for collecting virus particles from exhaled breath.

In an example diagnostic device 405 used for detection of captured virus particles, the resin beads in the packed bed column may be washed to remove any background material after exhaled breath samples is pulled through a sample capture element (for e.g., 101A, 101B, 201, or 301) following a predetermined breath sample collection protocol. The viral particles adsorbed in the packed bed column may be eluted or extracted using high concentration of acid solutions, including one or more of about 12.5% acetic acid, about 5% TFA, about 5% formic acid, or about 10% HCl, into the hot acid digestion chamber to generate the characteristic peptides. The peptide samples may be mixed with MALDI matrix and deposited onto as suitable substrate or plate for MALDI TOFMS analysis. In some implementations, the samples may also be deposited on a suitable substrate or disk that is precoated with MALDI matrix.

The disclosed example systems and methods may be used to establish a baseline of protein, metabolite, and lipids signatures in exhaled breath, which may then be used during to differentiate between the exhaled breath of patients with various respiratory infections and offer a powerful diagnostic tool for disease detection based on the analysis of aerosolized non-volatile biomaterial in exhaled breath.

In some implementations, a method for predicting a respiratory tract infection (“RTI”) may include selectively capturing truncated proteoforms in the exhaled breath aerosols produced by each patient using any one of the sample capture elements previously described, and removably connected to the exhaled air tubing of a ventilator, extracting the truncated proteoforms from the packed bed column into one or more collected liquid samples corresponding to each patient, analyzing the one or more collected liquid samples comprising truncated proteoforms using mass spectrometry to obtain raw mass spectra, identifying a statistically significant subset of the truncated proteoforms characteristic of the RTI, and predicting the presence of RTI using at least one of calculating a composite score representative of the statistically significant subset of the truncated proteoforms and calculating the area under the curve (AUC) of the receiver operating characteristic curve (ROC) representative of the statistically significant subset. The statistically significant subset of the class of truncated proteoforms may include at least one of CO6A3 (amino acid 2781-2792), CYTA (2-17), DEN2B (628-637), IRAK4 (121-130), MMP9 (673-691), and PHTF2 (271-285). Additional details are disclosed in commonly-owned U.S. patent application Ser. No. 17/827,708 and U.S. patent application Ser. No. 17/886,443, which are incorporated by reference herein in each of their entireties.

In some implementations, enzymes including proteases found in aerosolized exhaled breath may be captured using any one of the sample capture elements and systems previously described herein for detection of a respiratory tract infection. In some implementations, neutrophil elastase proteolysis activity of a substrate-based probe using captured neutrophil elastase (“NE”) in exhaled air may be examined using MALDI-TOFMS to detect respiratory tract infections. NE is characterized by a molecular weight of about 29 kDa and is a serine protease that plays an important role in neutrophil-mediated bacterial killing.

NE kills directly invading pathogens and fine tunes host inflammatory response for better pathogen eradication. NE activity can result in extensive lung tissue damages potentially leading to organ failure and death making inflammatory diseases a major health concern. Clinical studies have shown that an elevated concentration of NE correlates to acute lung injury, development of cystic fibrosis symptoms as well as chronic obstructive pulmonary disorders and bronchiectasis. See Jugniot et al. (2019), Choi, J-A. (2023).

Diagnostic assays including ELISA may be used to quantify NE from biological fluid samples. However, such antibody-related techniques yield information on total protease amount but lack the ability to differentiate between active and inactive enzyme forms. Clinical symptoms are often undetectable at an early stage of a disease, making diagnosis using antibody-related techniques even more challenging. Reliable assays to detect NE proteolytic activity as an indicator of RTI are needed. Conventional assays for measuring neutrophil elastase (“NE”) levels in clinical samples have been limited due to low concentrations of NE, which makes detection challenging. This limitation is particularly pronounced in human exhaled breath, where biomolecules are present at parts per billion (ppb) levels. To overcome this challenge, a substrate that exhibits high sensitivity to NE as a reporter may be needed. Utilizing a NE-specific responsive substrate allows for signal amplification, which enables the detection and quantification of NE in clinical samples, including human exhaled breath, with enhanced sensitivity. This approach provides a more accurate assessment of NE regulation under various disease conditions.

Substrate-based enzyme sensitive probes or sensors may be used to monitor NE activity using optical imaging, for example fluorescence or UV imaging. In these protease-sensitive probes for optical imaging a fluorescent or chromogenic molecule is bound to a peptide. Spectroscopic properties will be altered upon proteolysis by for example, NE. Substrate-based probes of the type N-methoxysuccinyl-Ala-Ala-Pro-Val-AMC or N-methoxy-succinyl-Ala-Ala-Pro-Val-pNA may be used to quantify NE and as a marker in inflammatory lung diseases. See Jugniot et al. (2019). In these probes, Amino-Methyl Coumarin (AMC, fluorophore) or p-Nitro-Anilide (pNA, chromophore) may be attached to the peptide and cleaved from the substrate during proteolysis by NE.

In contrast to fluorescence or UV imaging, MALDI-TOF mass spectrometry can examine and measure a characteristic mass spectral shift specific to a substrate-based probe during NE proteolysis. This unique feature allows for the development of multiplexing assays, where multiple probes, each having a different specific sensitivity to proteolysis activity, may be configured to target a specific protease, or a variety of proteases, including NE, trypsinogen, serine protease 22, Kallikreins, or matrix metalloproteinases matrix metalloproteinases (“MMPs”). MMPs are multi-functional enzymes that have the capacity to regulate both acute and chronic inflammation. They are also involved in cell repair and the remodeling of tissues. This multiplexing capability is not achievable with fluorescent-based assays. Additionally, mass spectrometry offers increased specificity through accurate mass measurements, and thereby reduces the risk associated with false positives during diagnosis.

Substrate-based enzyme-sensitive probes may be configured as polymer-peptide conjugate probe in which the peptide-fluorophore/chromophore probe is coupled to a polymer backbone. FIG. 5 shows a schematic diagram of polymer-peptide conjugate probe 500 including cleavable small molecule fluorophores or chromophores. A number of small molecules (flurophores or chromophores) 503 may be each coupled via peptide substrates 502, which are anchored to a polymer backbone or scaffold 501. The small molecules 503 are cleavable during proeteolysis, for example, in the presence of NE. During optical imaging, the abundance of small molecules disposed in proximity to each other may cause quenching of fluorescence, which is restored upon proteolysis.

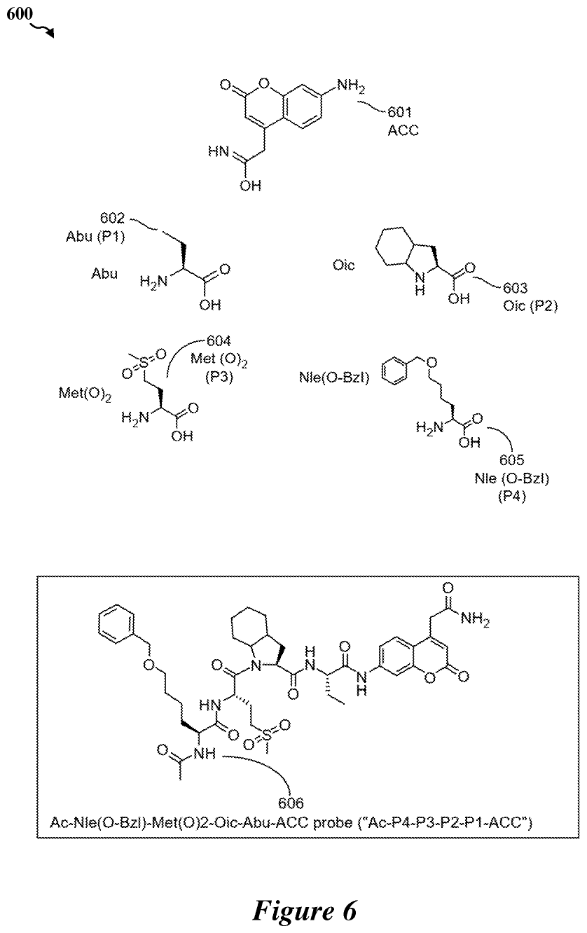

FIG. 6 shows the chemical structure of an example substrate-based probe 600 for human NE proteolysis. The substrate-based probe 600 as described by Kasperkiewicz et al. (2014) includes a peptide substrate 600 including unnatural amino acids for human NE detection of the form “Ac-P4-P3-P2-P1” to which a cleavable molecule may be coupled at the P1 peptide. In the above nomenclature, the prefix “Ac” refers to refers to the acetylation of the N-terminus of the peptide. Prior to peptide synthesis, the N-termini and amino acid side chains are “protected” with chemical groups that block nonspecific reactions during synthesis. The substrate-based probe Ac-Nle(O-Bzl)-Met(O)₂-Oic-Abu-ACC reportedly showed optimal fluorogenic properties for NE proteolysis. The cleavable molecule included 7-amino-4-carbamoylmethyl coumarin 601 (“ACC”) as the reporting fluorophore. The preferred P1 substrate substituent was “Abu” having a chemical structure 602. The preferred P2 substrate substituent with specificity for NE proteolysis was octahydro-1Hindole-2-carboxylic acid (“Oic”) having a chemical structure 603. The preferred P3 substrate substituent for NE proteolysis was methionine dioxide (“Met(O)₂”) having a chemical structure 604. The preferred P4 substrate substituent was Nle(O-Bzl) having a chemical structure 605. The chemical structure of the substrate-based probe Ac-Nle(O-Bzl)-Met(O)₂-Oic-Abu-ACC is shown at 606.

While the substrate-based probes for examining human NE proteolysis as described above may be used for fluorescence imaging, they are not suitable for top-down analysis and monitoring of human NE proteolysis using MALDI-TOFMS. In MALDI-TOFMS analysis, a substrate-based probe having an appropriate mass that falls with the optimized mass range of MALDI-TOFMS, which typically ranges from about 800 Da to about 20,000 Da, is required. Molecules with a mass below 800 Da, or in some instances, below 1500 Da may experience signal interference from matrix signals and may not be accurately characterized. Similarly, with an increase in the mass of substrate-based probes, sensitivity may dramatically decrease. Therefore, careful consideration of substrate-based probes having suitable masses are critical for MALDI-TOFMS for examining human NE proteolysis. As such, the conjugate probes previously described are not suitable for MALDI-TOFMS analysis of NE proteolysis.

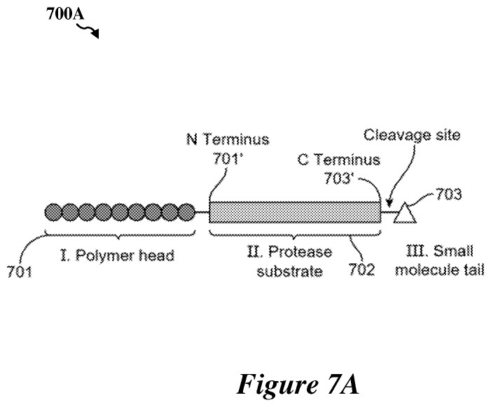

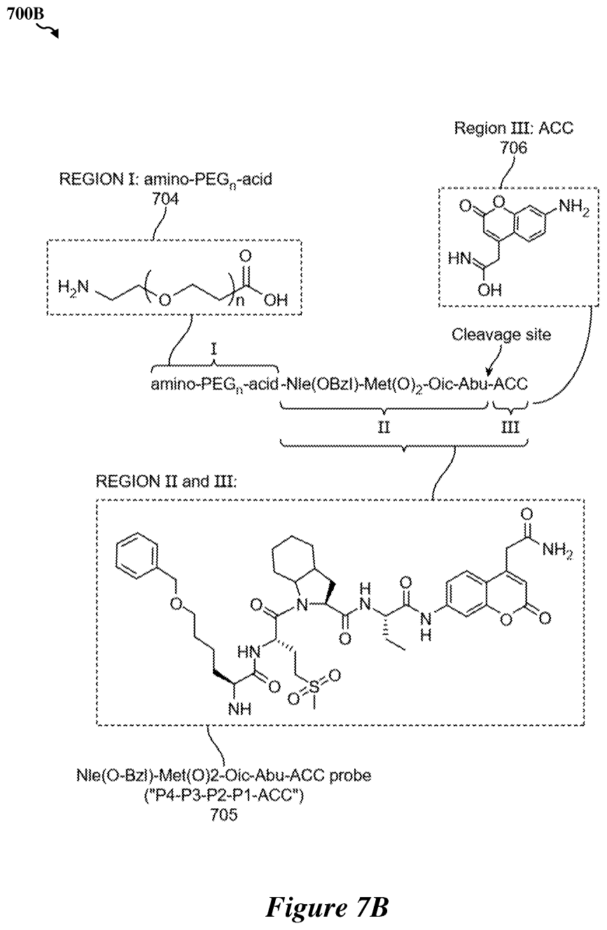

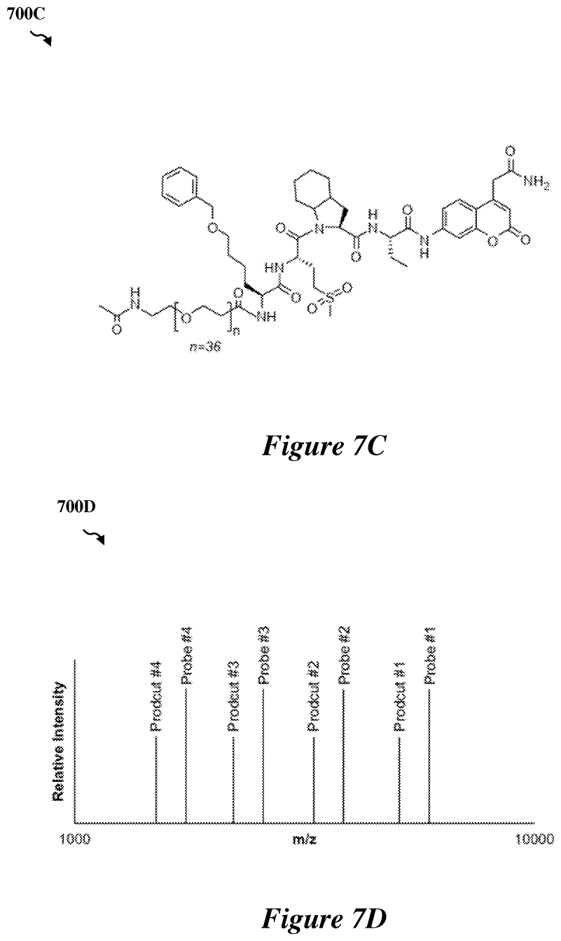

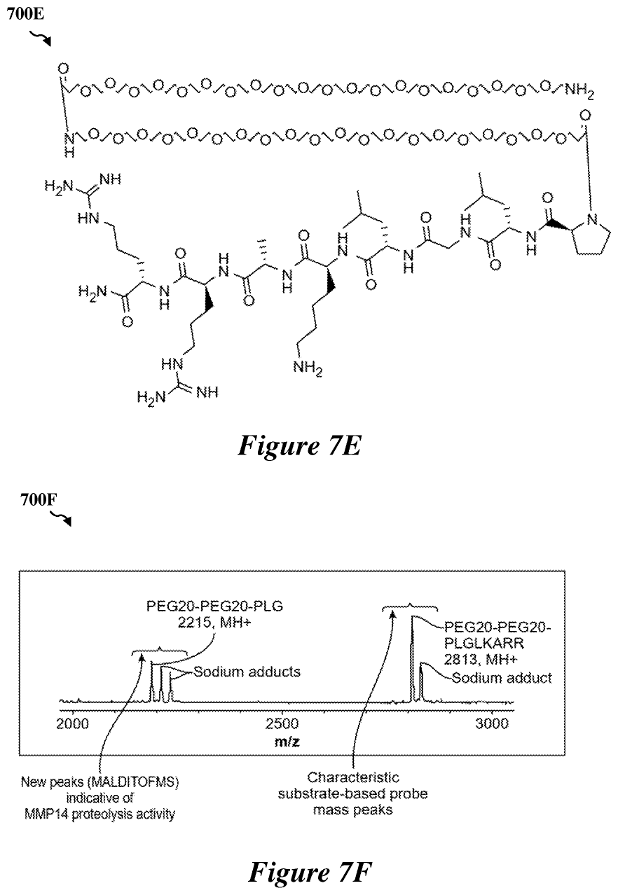

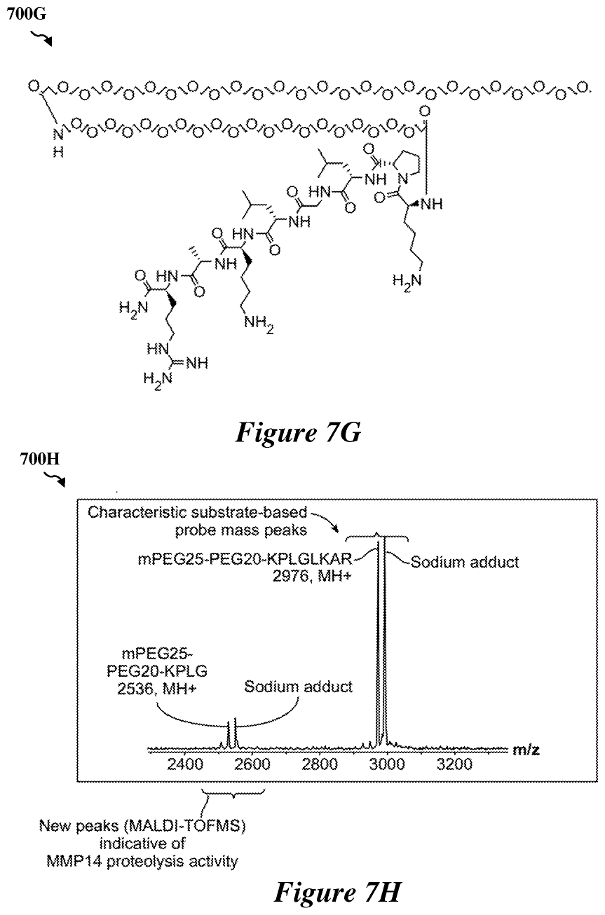

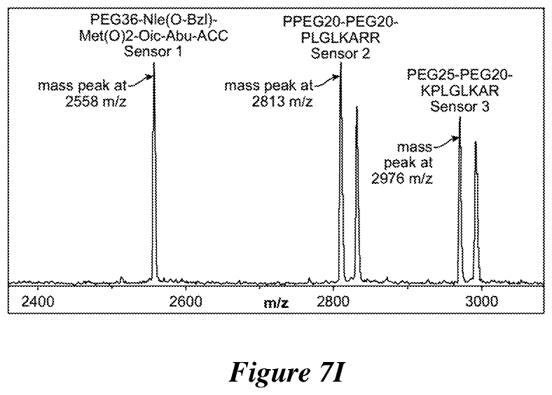

In some implementations, substrate-based probes for examining human NE proteolysis using MALDI-TOFMS may include a polymer head coupled to a protease substrate at the N terminus of the substrate and including a molecule tail coupled the C-terminus of the substrate. FIG. 7A shows a schematic diagram of an example substrate-based probe 700A for examining human NE proteolysis using MALDI-TOFMS, according to some implementations. As shown in FIG. 7A, an example substrate-based probe may include a polymer head 701 (region I) coupled to a protease substrate 702 (region II) at a head region (N terminus) 701′ of the protease substrate, and an NE-cleavable molecule 703 (region III) coupled to the protease substrate 702 at a tail region 703′ (C terminus). In some implementations, the polymer head 701 may include polyethylene glycol (PEG, Amino-PEG36-acid; CAS: 196936-04-6; catalog #: BP-22577; BroadPharm®, San Diego, CA). The PEG polymer head may include between about 20 to about 210 repeating units in a PEG chain. In some implementations, the PEG head 701 may include about 36 repeating units. The repeating units in the PEG polymer head 701 may be expressed as H—(O—CH₂—CH₂)ₙ—OH where “n” represents the number of repeating units. In some implementations, the polymer head may include amino-PEGₙ-acid, where the number of repeating units may be between about 20 and about 210.

In some implementations, the protease substrate 702 (Region II) may include a chemical compound, which may include natural amino acids or unnatural amino acids, and configured to be specific and sensitive to the proteolytic activity of NE. In some implementations, the NE substrate 702 may include the unnatural amino acid sequence Nle(O-Bzl)-Met(O)₂-Oic-Abu. Other amino acids may be considered for use as protease substrates as long as they do not compromise the sensitivity and specificity of the probe in detecting NE activity.

In some implementations, molecule 703 (region III) may include 7-amino-4-carbamoylmethyl coumarin (“ACC”; CAS number: 296236-23-2), as previously described herein.

FIG. 7B shows a schematic diagram of a substrate-based probe 700B for examining human NE proteolysis using MALDI-TOFMS according to some implementations. FIG. 7B also shows the chemical structures of the constituent regions of the substrate-based probe. As shown in FIG. 7B, an example substrate-based probe for examining human NE proteolysis using MALDI-TOFMS may include the general structure:

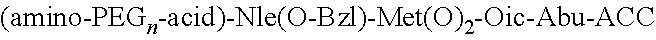

The amino-PEGₙ-acid head (Region I) 704 is coupled to the N-terminus of the Nle(O-Bzl)-Met(O)₂-Oic-Abu protease substrate (Region II) and the cleavable molecule (Region III) 706 is coupled to the C terminus of the protease substrate. The chemical structure 705 corresponding to Regions II and III is shown FIG. 7B. In some implementations, the polymer head including a polyethylene glycol (PEG) chain may include a plurality of repeating units of PEG, wherein the number of repeating units has a value (n) that is between about 20 and about 210. In some other implementations “n” has a numeric value of about 36. The chemical structures of the polymer head (Region 1) 704 and that of the molecule tail ACC (Region III) 706 are also shown in FIG. 7B.

FIG. 7C shows the chemical structure of an example substrate-based probe 700C for examining human NE proteolysis using MALDI-TOFMS, according to some implementations. As can be seen, the compound amino-PEG₃₆-acid is linked to the NE substrate at the N terminus and the cleavable molecule ACC is linked at C terminus of the substrate. The substrate's N-terminus is covalently bound to amino-PEG36-acid (polyethylene glycol) to align the sensor's mass within MALDI-TOF MS's optimal detection range of between about 1500 m/z (or Da) and 20000 m/z (or Da).

In MALDI-TOFMS examination of NE proteolysis using the example substrate-based probes as described above, the substrate-based probe may be characterized by a first MALDI-TOFMS mass spectra including one or more characteristic mass peaks (m/z). The substrate-based probe may be configured to disengage the tail molecule from its structure as a result of NE proteolysis activity and is characterized by a second MALDI-TOFMS mass spectra that is different from the first mass spectra, the second mass spectra including one or more new mass spectra peaks (m/z) indicative of NE proteolysis activity. In some implementations using example substrate-based probe 700C, the characteristic mass spectral peak (m/z) in the first MALDI-TOFMS mass spectra may be observed at about 2557 m/z. In some implementations, new mass spectral peaks (m/z) in the second MALDI-TOFMS mass spectra is observed at between about 2357 m/z and about 2379 m/z. In some implementations, the spectral shift characterized by the mass difference (Δm/z) between the characteristic mass spectral peaks (m/z) in the first MALDI-TOFMS mass spectra and the new mass spectra peaks (m/z) in the second MALDI-TOFMS mass spectra may be between about 175 and about 200. In some implementations, the sensitivity of the substrate-based probe to human NE in clinical samples using MALDI-TOFMS may be less than about 0.1 pM (pico-molar).

In some implementations, the polymer head (shown as region I in FIG. 7B) may be selected to tune the molecular weight of the substrate-based probe for monitoring or investigating more than one enzyme activity simultaneously to provide multiplexing capabilities. In some implementations, an example substrate-based probe with a mass range of about 1000 m/z to about 3000 m/z may include polyethylene glycol (“PEG”) repeating units, where the number of repeating units may be between about 3 and about 47. In some implementations, an example substrate-based probe with a mass range of about 3000 m/z to about 5000 m/z may include polyethylene glycol (“PEG”) repeating units, where the number of repeating units may be between about 48 and about 94. In some implementations, an example substrate-based probe with a mass range of about 5000 m/z to about 10000 m/z may include polyethylene glycol (“PEG”) repeating units, where the number of repeating units may be between about 95 and about 210. Accordingly, example substrate-based probes before and after proteolysis by proteases in exhaled air samples and other samples may be characterized by distinct masses (m/z) to prevent overlapping spectral peaks in MALDI-TOF mass spectra.

In some other implementations, the polymer head (shown as region I in FIG. 7B) may include one or more of polypropylene, polyethylene, polystyrene, polyvinyl chloride (“PVC”), polyurethane, polyacrylamide, polycarbonate, polyethylene terephthalate (“PET”), poly(methyl methacrylate) (“PMMA”), or polyvinyl alcohol (“PVA”). In some other implementations, the polymer head (shown as region I in FIG. 7B) may include chemical groups including one or more of peptides, small organic molecules, lipids, nucleic acids, or sugars that provide a compatible mass range for MALDI-TOFMS analysis.

The example systems and methods described herein are not necessarily limited to diagnosis of respiratory infections using MALDI-TOFMS. Lung cancer, for example, may also release NE or other proteases such as MMPs into the peripheral lung fluid, and NE and other proteases would be readily detected by any one of the systems and methods disclosed herein. Additionally, because blood comes into intimate contact with the alveolar lining in the lungs, NE biomarker of infection and cancer in other parts of the body (beyond the lungs) may be transferred across the alveolar lining and into the peripheral lung fluid, and thus, may be detected by the capture of non-volatile biomaterial or organics in EBA and analysis using MALDI-TOFMS. Accordingly, the scope of the invention is not limited to the detection and diagnosis of respiratory diseases.

In some implementations, clinical samples for protease analysis using the example substrate-based probes may include a sample including proteases in one or more of a sputum sample, an endotracheal tube sample, a bronchoalveolar lavage (“BAL”) sample, a blood sample, a fecal sample, or a homogenization sample of a tissue or semisolid biological sample. sample. Tissue homogenization is generally the process of breaking down tissues to form a suspension or emulsion of tissue solids, proteins and fluid, creating a suspension of tissue cellular fragments obtained after the tissue is homogenized, lysed, sonicated or digested. In some implementations, the tissue samples may include a mucosal biopsy sample including one or more of a lung mucosal biopsy sample, a colon mucosal biopsy sample, a rectal mucosal biopsy sample, an esophagus mucosal biopsy sample, or an oral mucosal biopsy sample. In some implementations, the tissue sample may include one or more of a lung sample, a liver sample, an oral biopsy sample, a stomach sample, an esophagus sample, a bone marrow sample, a colon sample, or an intestine sample. The example systems and methods disclosed above may also be used for predicting and diagnosing other diseases by capturing one or more other proteases in exhaled breath aerosols. The products of proteolysis of a suitable substrate-probe using captured proteases may be examined using MALDI-TOFMS (top-down analysis) to detect respiratory tract infections.

In some implementations, the example substrate-based probes as previously described herein, may be configured for multiplexing assays. As previously described, in multiplexing assays, a plurality of probes each with different specific masses may be configured to target various proteases, including NE, trypsinogen, serine protease 22, Kallikreins, or matrix metalloproteinases (“MMPs”). In some implementations, the molecule tail (region III as shown in FIGS. 7A-7B) in example substrate-based probes may be tuned to provide characteristic mass spectral shifts indicative of proteolysis. In some implementations, the molecule tail (region III as shown in FIGS. 7A-7B) may include volatile organic compounds including limonene. Limonene is a cyclic monoterpene with a formula C₁₀H₁₆ and may be known as 1-methyl-4-(1-methylethenyl)-cyclohexene.

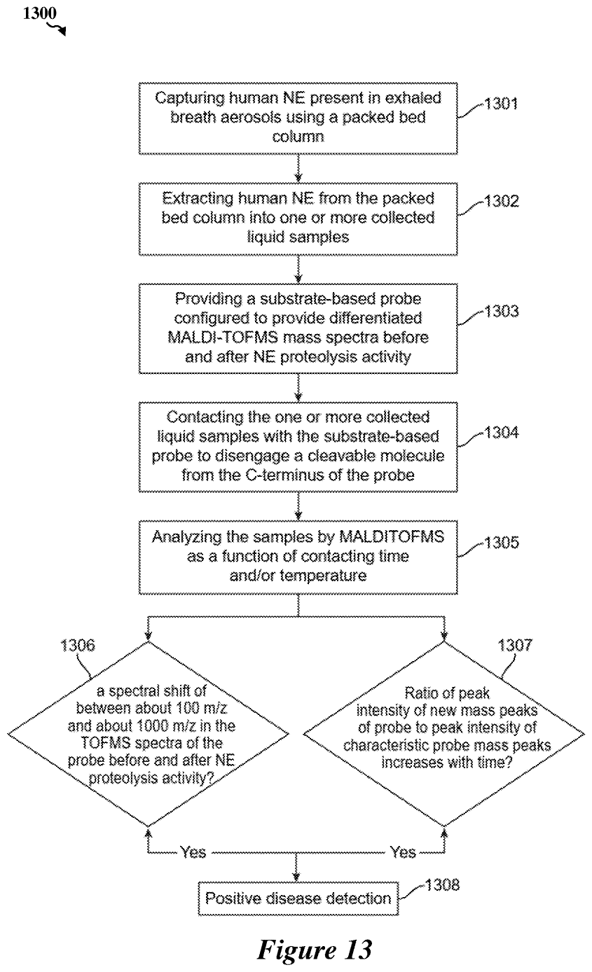

FIG. 13 shows a schematic diagram of an example method 1300 for detecting disease using neutrophil elastase (NE) proteolysis activity of NE captured from exhaled breath aerosols, according to some implementations. Example method 1300 may include capturing human NE present in exhaled breath aerosols using a packed bed column at 1301, extracting human NE from the packed bed column into one or more collected liquid samples at 1302, and providing any one of the previously described substrate-based probes at 1303. The example substrate based probes may be characterized by a first MALDI-TOFMS mass spectra including one or more characteristic mass peaks (m/z), wherein the substrate-based probe is configured to disengage a cleavable molecule from its structure as a result of NE proteolysis activity and is characterized by a second MALDI-TOFMS mass spectra that is different from the first mass spectra, the second mass spectra including one or more new mass spectra peaks (m/z). Method 1300 may continue at 1304 with contacting the one or more collected liquid samples with the substrate-based probe during one or more of a predetermined contacting time or predetermined contacting temperature to disengage the cleavable molecule. The example method 1300 may continue at 1305 with analyzing the sample using MALDI-TOFMS at different contacting times. The example method may continue with determining the presence of a disease at 1308 if one or more of the following is observed: at 1306, a spectral shift characterized by the mass difference (Δm/z) between the one or more characteristic mass spectral peaks (m/z) in the first MALDI-TOFMS mass spectra and the one or more new mass spectra peaks (m/z) in the second MALDI-TOFMS mass spectra is between about 100 m/z and about 1000 m/z, or at 1307, in the second MALDI-TOFMS mass spectra, a ratio of the mass spectral peak intensity associated with one or more new mass spectra peaks (m/z) to that associated with the one or more characteristic mass spectra peak (m/z) increases as a function of contacting time.

In the example method 1300, capturing NE in exhaled breath aerosols at 1301 may include capturing, using a packed bed column, exhaled breath aerosols from a patient breathing using a ventilator in an intensive care unit of a hospital. In some implementations, the packed bed column may include one or more of resin beads having C18 functional groups on the surface, cellulose beads having sulfate ester functional groups on the surface, or mixtures thereof. In some implementations, the resin beads and cellulose beads may have a nominal diameter of at least about 20 μm. In some implementations, the resin beads and cellulose beads may have a nominal diameter of between about 40 μm and about 150 μm.

In the example method 1300, extracting NE from the packed bed column at 1302 may include flushing the packed bed column with one or more solvents to produce one or more collected liquid samples including NE. In some implementations, the one or more solvents may include one or more of acetonitrile, methanol, trifluoro acetic acid (TFA), or isopropanol (IPA), the remaining being water. In some implementations, the one or more solvents may include between about 50 vol % and about 70 vol % acetonitrile in water, between about 50 vol % and about 70 vol % isopropanol in water, or between about 0.05 vol % TFA in water.

In the example method 1300, providing a substrate-based probe at 1303 may include providing the substrate-based probe 700C as shown in FIG. 7C. In the example substrate-based probe 700C, wherein the cleavable molecule may include ACC, as previously described herein. Using substrate-based probe 700C, the characteristic mass spectral peak (m/z) in the first MALDI-TOFMS mass spectra may be observed at about 2557 m/z. The new mass spectral peaks (m/z) in the second MALDI-TOFMS mass spectra may be observed at between about 2357 m/z and about 2379 m/z. Accordingly, a spectral shift characterized by the mass difference (Δm/z) between the characteristic mass spectral peaks (m/z) in the first MALDI-TOFMS mass spectra and the new mass spectra peaks (m/z) in the second MALDI-TOFMS mass spectra is between about 175 and about 200.

In the example method 1300 and referring to FIG. 13, contacting the one or more collected liquid samples with the substrate-based probe at 1304 may include incubating at between about 20° C. and about 37° C. for at least about 10 min. Example method 1300 may be used for detecting diseases including one or more of a respiratory tract infection, lung cancer, or any other lung disorders and cancer types that release NE into lung fluids and subsequently transferable to exhaled breath aerosols.

In some implementations, contacting the one or more collected liquid samples with the substrate-based probe includes incubating at between about 20° C. and about 70° C. for between about 5 min. and about 10 min. to realize rapid detection of lower respiratory tract infections using the example substrate-based probes described herein. In some instances, the incubation period may decrease with increasing incubation temperature. Accordingly, the incubation period and temperature may be tuned to quickly generate the cleavage product resulting from proteolysis of rhNE using an example substrate-based sensor as described herein.

In some implementations, the example substrate-based probes and methods disclosed herein may be used for detecting proteolysis activity of other protease biomarkers, in addition to NE. In some implementations, the one or more proteases may include one or more of, trypsinogen, serine protease 22, Kallikreins, or matrix metalloproteinases (“MMPs”). In some implementations, a method for detecting proteolysis activity of one or more proteases captured from exhaled breath aerosols, may include capturing the one or more proteases present in exhaled breath aerosols using a packed bed column, extracting the one or more proteases from the packed bed column into one or more collected liquid samples, and providing a substrate-based probe characterized by a first MALDI-TOFMS mass spectra including one or more characteristic mass peaks (m/z), wherein the substrate-based probe is configured to disengage a cleavable molecule from its structure as a result of proteolysis activity and is characterized by a second MALDI-TOFMS mass spectra that is different from the first mass spectra, the second mass spectra including one or more new mass spectra peaks (m/z). The example method may continue with contacting the one or more collected liquid samples with the substrate-based probe during one or more of a predetermined contacting time or predetermined contacting temperature to disengage the cleavable molecule.

In some implementations, multiplexing assays may include more than two substrate-based probes. Multiplexing enables analyzing the activity of more than one proteases in a single assay, thereby enhancing the efficiency and effectiveness of diagnostic assays. FIG. 7D shows a schematic diagram of four different substrate-based probes associated with a multiplexing assay and their respective characteristic MALDI-TOFMS mass spectra before and after proteolysis of a specific protease in exhaled breath, according to some implementations. Distinct mass spectral shifts before and after proteolysis associated with each probe may be indicative of the presence of various proteases in the samples. As previously described, the presence of proteases may be viewed as biomarkers of various diseases.

In some implementations, an example method for detecting proteolysis activity of one or more proteases captured from exhaled breath aerosols in a multiplexing assay may include, capturing the one or more proteases present in exhaled breath aerosols using a packed bed column, extracting the one or more proteases from the packed bed column into one or more collected liquid samples and analyzing the one or more collected liquid samples using a plurality of substrate-based probes. Each probe may be characterized by a first MALDI-TOFMS mass spectra including one or more characteristic mass peaks (m/z), wherein each substrate-based probe is configured to disengage a cleavable molecule from its structure as a result of proteolysis activity and is characterized by a second MALDI-TOFMS mass spectra that is different from the first mass spectra, the second mass spectra including one or more new mass spectra peaks (m/z). The example multiplexing assay may include contacting the one or more collected liquid samples with the one or more substrate-based probes during one or more of a predetermined contacting time or predetermined contacting temperature to disengage the cleavable molecule. In some implementations, the one or more proteases may include one or more of NE, trypsinogen, serine protease 22, Kallikreins, or matrix metalloproteinases (“MMPs”). In some implementations, the capturing step may include drawing exhaled breath aerosols into the packed bed column using a pump. In some implementations, the capturing step may include drawing exhaled breath aerosols into the packed bed column using a pump, wherein the packed bed column is fluidly connected to an exhaled air tubing of a ventilator used to assist the breathing of an intubated patient. In some implementations, an example multiplexing assay associated with exhaled breath analysis using a plurality of substrate-based probes may include detecting the proteolysis activity of human neutrophil elastase (“HNE”) or MMPs. In some instances, MMPs may include matrix metalloproteinase MMP8 or matrix metalloproteinase MMP9. In some instances, the plurality of substrate-based probes in an example multiplexing assay associated with exhaled breath analysis may include three substrate-based probes, as described below:

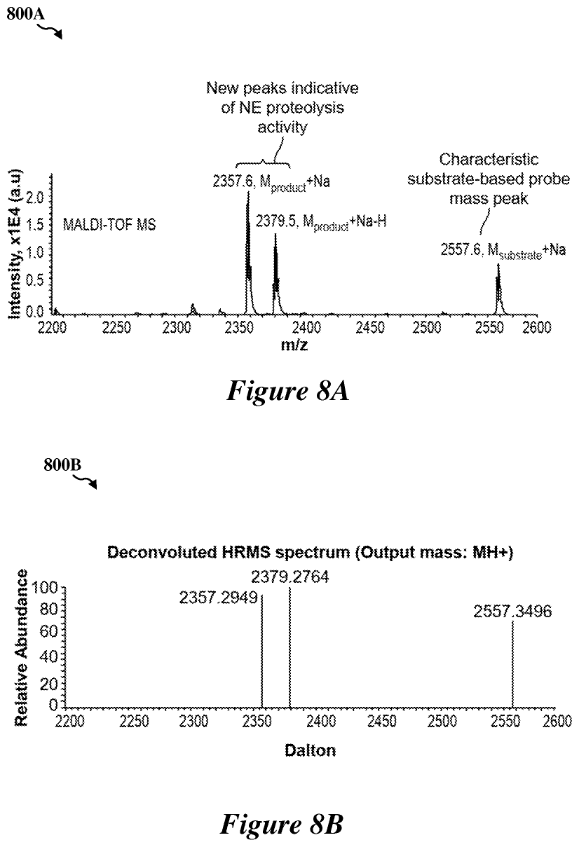

(a) PEG36-Nle(O-Bzl)-Met(O)2-Oic-Abu-ACC (“first probe”) configured to target proteolysis of HNE in exhaled breath. The chemical structure of the example first probe was previously described with reference to FIG. 7C. Referring to FIG. 8A discussed below, the mass peak at about 2558 m/z is associated with the intact substrate-based first probe.

(b) PEG40-Pro-Leu-Gly-Leu-Lys-Ala-Arg-Arg (“second probe”, also referred to herein as PEG20-PEG20-PLGLKARR, with PLGLKARR representing a peptide/amino acid sequence) configured to target proteolysis of MMPs, in particular MMP-8. Example MMPs include one or more of MMP-8 (also referred to as collagenase-2 or neutrophil collagenase), MMP-9 (also referred to as gelatinase B), or MMP-14 (also referred to as membrane-bound matrix metalloproteinase or MT1-MMP). The example second probe may be represented by the molecular formula C₁₂₆H₂₄₆N₁₈O₅₀ and may be characterized by a molecular weight of about 2813 Da. The chemical structure 700E of the example second probe is shown in FIG. 7E. FIG. 7F shows a representative MALDI-TOFMS spectrum 700F of MMP14 proteolysis reaction sample solution using a substrate-based probe, according to some implementations. The substrate-based probe included the second probe PEG20-PEG20-PLGLKARR. The sensor was incubated with a recombinant human MMP-14 (R&D Systems, Inc., Catalog #: 918-MP). Referring to FIG. 7F, the mass peak at about 2813 m/z is associated with the intact substrate-based probe. The mass peak at about 2215 m/z is associated with the cleaved product of MMP-14 as a result of proteolysis. LKARR is removed or disengaged from the substrate during MMP-14 proteolysis. As can be seen, mass peaks corresponding to sodium adducts were also observed, and

(c) PEG45-Lys-Pro-Leu-Gly-Leu-Lys-Ala-Arg (“third probe”, also referred to herein as PEG25-PEG20-KPLGLKAR, with KPLGLKAR representing a peptide/amino acid sequence) configured to target proteolysis of MMPs, in particular MMP9. Example MMPs include one or more of MMP-8, MMP-9, or MMP-14. The example third probe may be represented by the molecular formula C₁₃₅H₂₆₃N₁₅O₅₅ and may be characterized by a molecular weight of about 2976 Da. The chemical structure 700G of the example third probe is shown in FIG. 7G. FIG. 7H shows a representative MALDI-TOFMS spectrum 700H of a MMP proteolysis reaction sample solution using a substrate-based probe, according to some implementations. The substrate-based probe included the third probe PEG25-PEG20-KPLGLKAR. The sensor was incubated with a recombinant human MMP-14 (R&D Systems, Inc., Catalog #: 918-MP). Referring to FIG. 7H, the mass peak at about 2976 m/z is associated with the intact substrate-based probe. The mass peak at about 2536 m/z is associated with the cleaved product of MMP-14 as a result of proteolysis. LKAR is removed or disengaged from the substrate as a cleaved product during MMP14 proteolysis. As can be seen, mass peaks corresponding to sodium adducts were also observed.

In some implementations, an example multiplexing assay associated with exhaled breath analysis may include the example first probe PEG36-Nle(O-Bzl)-Met(O)2-Oic-Abu-ACC, the example second probe PEG20-PEG20-PLGLKARR, and the example third probe PEG25-PEG20-KPLGLKAR, as previously described herein. Target proteases may include HNE and MMPs. FIG. 7I shows a representative MALDI-TOFMS spectrum 700I of a solution including three different substrate-based probes for a multiplexing assay associated with exhaled breath analysis, according to some implementations. In some instances, the concentration of each probe may be about 200 micromolar (pM) in a reaction buffer including 50 mM Tris, 1 M NaCl, 0.05% (w/v) Brij-35. The reaction buffer solution may be characterized by a pH of about 7.5. Referring to FIG. 7I, the first probe (also referred to herein as sensor), second probe, and third probe are characterized by mass peaks at about 2558 m/z, about 2813 m/z, and about 2976 m/z.

In some implementations, an example operation for predicting a lower respiratory tract infection (“LRTI”) may include capturing aerosolized non-volatile particles including one or more proteases present in exhaled air by routing exhaled air from a patient to an aerosolized non-volatile particle collection system including a packed bed column. In some instances, prior to capturing the aerosolized non-volatile particles including one or more proteases, the example operation may include activating the packed bed column. In some instances, the example operation may continue with extracting the one or more proteases from the packed bed column into one or more collected liquid samples. In some examples, the example operation may continue with contacting a first aliquot of the one or more collected liquid samples with a plurality of substrate-based probes (also referred to herein as a multiplexing assay) during one or more of a predetermined contacting time or predetermined contacting temperature to disengage a cleavable molecule associated with each of the substrate-based probes and producing one or more reacted liquid samples associated with each of the one or more collected liquid samples. In some other examples, an example operation may continue with estimating the concentration of the one or more proteases in the one or more reacted liquid samples by analyzing a first aliquot of the one or more collected liquid samples using MALDI-TOFMS. In some instances, the example operation may continue with predicting the presence of an LRTI if a concentration associated with the one or more proteases in the one or more reacted liquid samples is equal to or greater than a cut-off threshold concentration associated with the one or more proteases. In some other instances, the example operation may continue with identifying a causative pathogen associated with the LRTI by analyzing a second aliquot of the one or more collected liquid samples, using a quantitative polymerase chain reaction (“qPCR”, also referred to herein as real-time PCR) assay. q-PCR may be used to detect trace levels of genomic material with high sensitivity and specificity.

In some implementations, a second aliquot of the one or more collected liquid samples may be analyzed using a qPCR assay to identify the causative pathogen if the proteolysis activity associated with first aliquot of the one or more collected liquid samples, as described above, indicates the presence of an LRTI. This sequential analysis method may not only predict or confirm the presence of an active LRTI, but may also provide pathogen-specific information, and offers a comprehensive solution for managing LRTI cases. As such, the example sequential analysis method described herein may be quick (for example, in the order of minutes), and characterized by high sensitivity and specificity compared to conventional diagnostic methods commonly used in hospitals, including bronchoalveolar lavage (“BAL”) culture, which relies on invasive specimen collection and often requires an analysis time of several days. In contrast, the sequential analysis method described above offers an analytical result by leveraging the analysis of non-invasive exhaled air samples (as collected liquid samples) and integrating rapid host response detection with pathogen identification.

In some implementations, an assay kit for detecting proteolysis activity of one or more proteases in a clinical sample may include one or more substrate-based probes as previously described herein. Each substrate-based probe may be characterized by a first MALDI-TOFMS mass spectra including one or more characteristic mass peaks (m/z), wherein each substrate-based probe is configured to disengage a cleavable molecule from its structure as a result of proteolysis activity and is characterized by a second MALDI-TOFMS mass spectra that is different from the first mass spectra, the second mass spectra including one or more new mass spectra peaks (m/z). In some implementations, the clinical sample may include a sample including proteases captured from exhaled breath aerosols. In some implementations, the clinical sample may include a sample including proteases in one or more of a sputum sample, an endotracheal tube sample, a bronchoalveolar lavage (“BAL”) sample, a blood sample, a fecal sample, or a homogenization sample of a tissue or semisolid biological sample. In some implementations, the tissue sample may include a mucosal biopsy sample including one or more of a lung mucosal biopsy sample, a colon mucosal biopsy sample, a rectal mucosal biopsy sample, an esophagus mucosal biopsy sample, or an oral mucosal biopsy sample. In some implementations, the tissue sample may include one or more of a lung sample, a liver sample, an oral biopsy sample, a stomach sample, an esophagus sample, a bone marrow sample, a colon sample, or an intestine sample. In some implementations, the one or more proteases may include one or more of NE, trypsinogen, serine protease 22, Kallikreins, or matrix metalloproteinases (“MMPs”).

In some implementations, each of the one or more substrate-based probes in the assay kit may include a protease substrate including one or more of natural amino acids or unnatural amino acids, the protease substrate including a head region, and a tail region, a polymer head coupled to the head region (N terminus) of the protease substrate and a tail molecule coupled to the tail region (C terminus) of the protease substrate. In some implementations, each substrate-based probe may be characterized by a first MALDI-TOFMS mass spectra including one or more characteristic mass peaks (m/z), and wherein the substrate-based probe is configured to disengage the tail molecule from its structure as a result of NE proteolysis activity, and is characterized by a second MALDI-TOFMS mass spectra that is different from the first mass spectra, the second mass spectra including one or more new mass spectra peaks (m/z) indicative of NE proteolysis activity. In some implementations, the cleavable molecule may include one or more of 7-amino-4-carbamoylmethyl coumarin (“ACC”), limonene, LKARR, or LKAR. In some implementations, the polymer head may include a polyethylene glycol (“PEG”) chain including a plurality of repeating units of PEG, wherein the number of repeating units is between about 20 and about 210. In some implementations, each of the one or more substrate-based probes may include a polymer head characterized by one or more of a different molecular weight or a characteristic MALDI-TOFMS mass spectra.

### EXAMPLES

### Example 1. Calibration of the Example NE Substrate-Based Probe 700C by Proteolysis Using Mouse Neutrophil Elastase

The ability of the example NE substrate-based probe 700C to produce enzyme reaction cleavage product as a function of time using recombinant enzyme recombinant mouse neutrophil elastase (rmELA2) was examined. For enzyme activation, about 1 μL of rmCatC stock (440 μg/mL) was combined with about 1 μL of 5 mM DTT in activation buffer and incubated at room temperature for 30 min. After incubation, this solution was combined with about 1 μL rmELA2 stock (440 μg/mL) diluted in 5.8 μL activation buffer and incubated for 2 h at 37° C. to activate rmELA2. The final concentration of each enzyme was about 50 μg/mL. To prepare the substrate-probe 700C, about 25 μL of the neutrophil elastase substrate 700C was diluted two-fold in 25 μL assay buffer. About 1 μL of the activated rmELA2 solution was diluted in 49 μL of assay buffer. Next about 50 μL of the substrate-probe 700C solution was added to the 50 μL of diluted activated rmELA2 to initiate the enzymatic proteolysis. Mass spectra at samples collected after incubation period of 0 min, 10 min, 30 min, 60 min, 120 min, and 180 min to examine the product/substrate ratio over time.

Activation buffer (50 mM MES, 50 mM NaCl, pH 5.5) and assay buffer (50 mM Tris, 1 M NaCl, 0.05% (w/v) Brij-35, pH 7.5) were obtained from Sigma Aldrich. Dithiothreitol (DTT) was also obtained from Sigma Aldrich. Recombinant mouse neutrophil elastase/ELA2 protein (rmELA2; Catalog #4517-SE) and recombinant mouse active cathepsin C/DPPI protein (rmCatC; Catalog #2336-CY), both with a stock concentration of 440 μg/mL, were obtained from R&D Systems, Inc (Minneapolis, MN). The two proteins were stored in 1 μL aliquots at −80° C.

For MALDI-TOFMS analysis of sample, about 1 μL of each reaction (proteolysis) sample solution was added to a Bruker MALDI plate spot plate (MSP 96 target ground steel). The MALDI plate was transferred to a heating block set to 60° C. until the spots were dry. The MALDI plate was removed from the heating block and 1.0 μL of α-Cyano-4-hydroxycinnamic acid (“CHCA”) MALDI matrix (9 mg/mL in 70% acetonitrile) was added to the sample spot. The MALDI plate was then transferred back to the heating block set to 60° C. until spots were dry. The samples were characterized using a Bruker AutoFlex MALDI-TOF instrument set in positive ion linear mode.

FIG. 8A shows a representative MALDI-TOFMS spectrum 800A of a NE proteolysis reaction sample solution after about 10 min of incubation or reaction time. The substrate-based probe (sensor) 700C was characterized by a mass of about 2557 m/z. The mass spectra of the substrate-based probe after NE proteolysis activity were characterized by two new spectral peaks, at about 2357 m/z and about 2379 m/z, with different sodium adducts. FIG. 8B shows a representative deconvoluted high-resolution mass spectra 800B of a reaction sample solution after about 10 min of incubation. High-resolution mass spectrometry (HRMS) was performed using a Thermo Scientific LTQ Orbitrap system in the positive ion mode. The analysis was carried out via direct infusion, and a mass resolution of 60,000 m/z was employed. As can be seen, MALDI-TOF mass spectra 800A is comparable to the high-resolution mass spectra 800B. The high-resolution mass spectra showed that the substrate-probe (sensor) has a mass of about 2557 (m/z) with a sodium adduct.

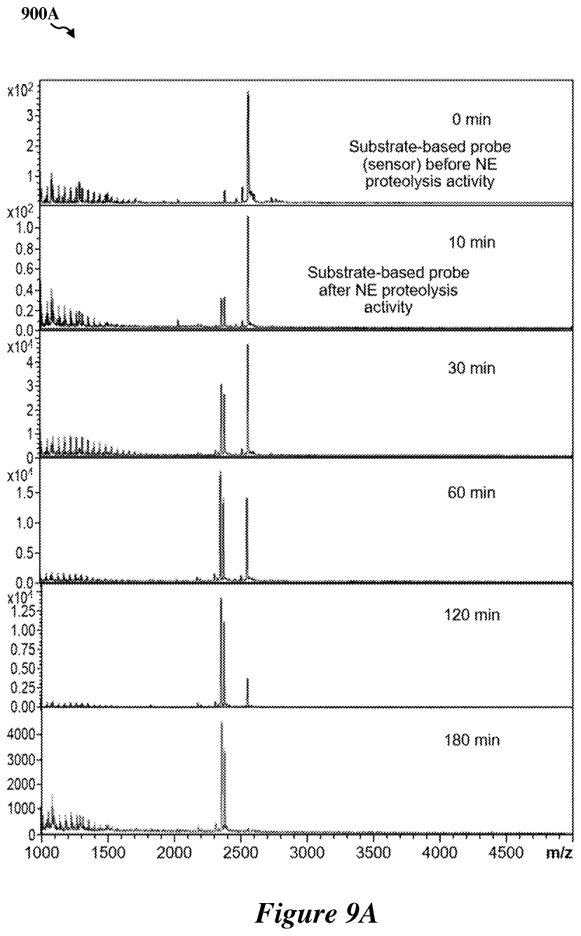

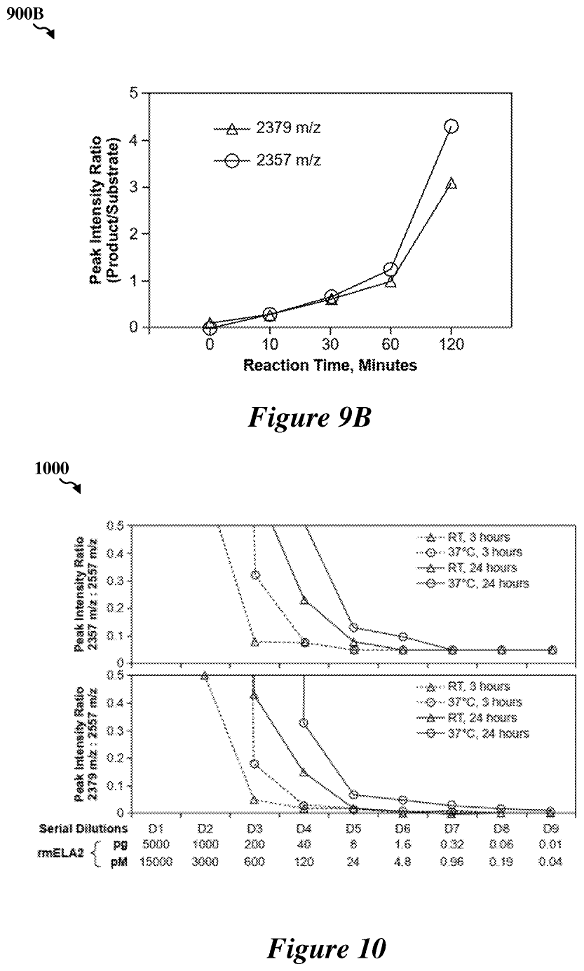

FIG. 9A shows representative MALDI-TOFMS mass spectra 900A of NE proteolysis reaction (with substrate-based probe 700C) of recombinant mouse neutrophil elastase samples as a function of incubation period, according to some implementations. The concentration of rmNE in the samples was about 15 pM. As can be seen, new substrate-based probe mass peaks associated with the cleavage products indicative of proteolysis activity were detected using MALDI-TOFMS even after only a 10-min incubation period. After an incubation period of about 180-min, the characteristic mass peak (at about 2557 m/z) associated with the substrate-probe (that is, the intact sensor peak) was not detected suggesting completion of the enzyme proteolysis reaction.

Additionally, quantification of the reaction kinetics associated with the proteolysis reaction in the presence of substrate-based probe 700C was examined by calculating the ratio of the mass spectral peak intensity associated with the cleavage product to the mass spectral peak intensity associated with the intact sensor. FIG. 9B shows a plot 900 B of representative ratio of the MALDI-TOFMS mass spectral peak intensity associated with one or more new mass spectra peaks (m/z) to that associated with the one or more characteristic mass spectra peak (at about 2557 m/z) as a function of reaction time, according to some implementations. The samples were associated with proteolysis reaction (with substrate-based probe 700C) of recombinant mouse neutrophil elastase as a function of incubation period during calibration. As can be seen, both the ratio of the peak intensity corresponding to the cleavage product mass peaks at 2357 m/z to that of the intact sensor, and the ratio of the peak intensity corresponding to the cleavage product mass peaks at 2379 m/z to that of the intact sensor, increased with an increase in incubation period indicative of strong correlation with NE proteolysis activity. The results also suggest that the intensity associated with the mass peak at 2357 m/z is a more reliable indicator for quantitative analysis because trace levels of the mass peak at 2379 m/z was detected in the sample before the onset of proteolysis, that is, at a reaction time of 0 min.

FIG. 10 shows a plot 1000 of the ratio of the MALDI-TOFMS mass spectral peak intensity associated with one or more new mass spectra peaks (m/z) to that associated with the one or more characteristic mass spectra peak (at about 2557 m/z) as a function NE concentration, according to some implementations. Tests were conducted to investigate the NE activity limit of the substrate-based probe 700C using recombinant enzyme recombinant mouse neutrophil elastase (rmELA2). For enzyme activation, 1 μL of rmCatC stock (440 μg/mL) was combined with 1 μL of 5 mM DTT in activation buffer and incubated at room temperature for 30 min. After 30 min. incubation, the solution was combined with 1 μL rmELA2 stock (440 μg/mL) diluted in 5.8 μL activation buffer and incubated for 2 h at 37° C. to activate rmELA2. This protocol resulted in a final concentration of 50 μg/mL for each enzyme. After 2 h incubation, 1 μL of the activated rmELA2 solution was diluted in 49 μL of assay buffer and was used to generate eight subsequent 5-fold serial dilutions in the assay buffer. To prepare the substrate-based probe, 50 μL of the substrate-based probe solution was diluted in 150 μL assay buffer.

To initiate the enzyme reaction (NE proteolysis), 5 μL of the substrate-based probe solution was added to 5 μL of each neutrophil elastase serial dilution (from 5 ng to 0.01 pg), yielding a total reaction volume of 10 μL. This protocol replicated for 4 different incubation conditions: 3 h at room temperature, 3 h at 37° C., 24 h at room temperature, and 24 hours at 37° C. Upon completion of each incubation period, NE proteolysis activity was examined using MALDI-TOFMS. At both incubation temperatures, and incubation periods, the ratio of the MALDI-TOFMS mass spectral peak intensity associated with one or more new mass spectra peaks (m/z) to that associated with the one or more characteristic mass spectra peak (at about 2557 m/z) decreased with the decrease in concentration of the recombinant mouse NE enzyme. The NE enzyme acts as an activation agent or catalyst for cleaving the substrate-based probe as previously described. As such, the concentration of NE does not change during the proteolysis reaction. The substrate-based probe 700C was found to be sensitive to proteolysis activity of NE concentration of less than about 0.1 picomolar (“pM”) suggesting that trace amounts of NE captured from exhaled air would be sufficient to detect an infection using the example substrate-based probe 700C.

### Example 2. Detection of RTI in Patients Breathing Through a Mechanical Ventilator by Capturing Neutrophil Elastase in Exhaled Air and Examining the Products of Neutrophil Elastase Proteolysis of a Substrate-Based Probe Using MALDI-TOFMS

Non-volatile organic aerosolized particles (including neutrophil elastase in patients with RTI) in exhaled air aerosols of 14 patients breathing through a ventilator (see FIG. 1B) were captured using sample capture element 101B. The patients were undergoing treatment at the Johns Hopkins Hospital Intensive Care Unit (“JHH ICU”). The captured non-volatile organics were extracted, and one or more collected liquid samples were reacted with substrate-based probe 700C. Neutrophil elastase proteolysis activity was examined using MALDI-TOFMS of substrate-based probe samples to detect respiratory tract infections. As previously described, the detection of new substrate-based probe peaks at about 2357 m/z and about 2379 m/z may be considered as indicators of NE proteolysis activity and subsequently, of lower tract respiratory infections (“LTRIs”) and MALDI-TOFMS predictions were confirmed using standard culturing methods.

The sample capture elements were stored at 4° C. until using for sample capture from the ventilator patients. For extraction of captured non-volatile organics from the sample capture elements, about 300 μL of 70% acetonitrile (v/v in water) was added to each column in a 2 mL Eppendorf tube. The sample capture elements were centrifuged at 2,000 RCF for about 5 min. Next, about 300 μL of LC-MS grade water was added into each sample capture element. The sample capture elements were kept at room temperature for about 10 min. and centrifuged at 2,000 RCF for about 5 min. The liquid in each sample capture element was removed and the liquid sample was vortexed (shaken using a vortex mixer) for about 30 s to mix the sample thoroughly. Each liquid sample was placed in a tube and sealed with a 0.2 μm filter and placed in a −80° C. freezer for 2 h. The samples were lyophilized overnight. After overnight lyophilization, samples were resuspended in 50 μL LC-MS grade water and vortexed.

About 23 μL of substrate-based probe 700C was combined with about 68 μL assay buffer. To initiate enzyme proteolysis, about 5 μL of the substrate-probe solution was added to about 5 μL of each of the 14 clinical samples and 2 control samples of 50 μL LC-MS water to provide a total reaction volume of 10 μL for each sample. The liquid mixture was then incubated for about 3 h at 37° C. and 24 h at 37° C. Upon completion of each incubation step, samples were analyzed using MALDI-TOFMS to examine neutrophil elastase proteolysis activity of the substrate-probe.

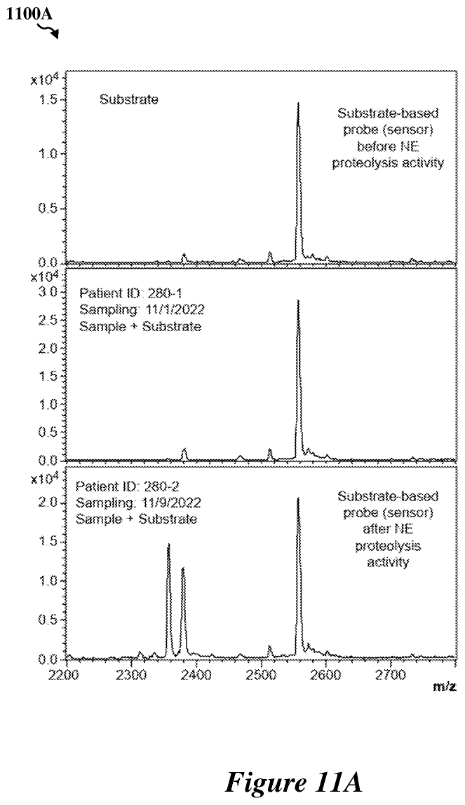

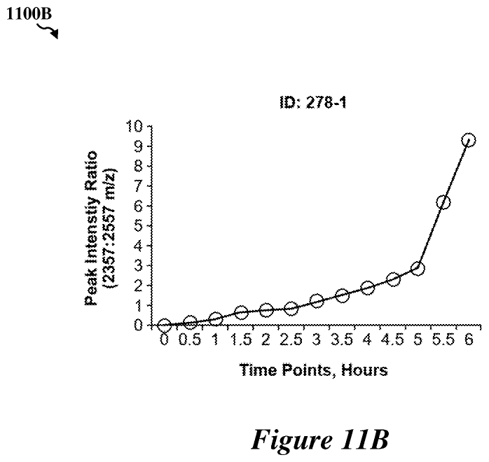

FIG. 11A shows MALDI-TOFMS mass spectra 1100A of an example substrate-based probe 700C under NE proteolysis of clinical samples collected from a patient breathing using a ventilator on different days, according to some implementations. Clinical samples 280-1 and 280-2 were collected from the same patient but on different days. Clinical sample 280-1 only shows trace levels of new peaks (m/z: 2357 to 2379). Sample 280-2 collected after about 8 days of sample 280-1, clearly shows significant new mass peaks associated with the substrate-based probe suggesting that the concentration of NE captured using sample capture element was higher in sample 280-2. This suggests upregulation of NE caused by an infection.

FIG. 11B shows a plot 11 OOB of MALDI-TOFMS peak intensity ratio of the peak intensity associated with new mass spectra peaks (2357 m/z) to that associated with the one or more characteristic mass spectra peak (about 2557 m/z) as a function of contacting time, according to some implementations. As can be seen, the peak intensity ratio increases with increase in incubation period. As such, the example substrate-based probes as disclosed herein, and MALDI-TOFMS analysis of the proteolysis of neutrophil elastase in captured EBA samples using the example substrate-based probes allows for rapid analysis of clinical diagnosis. The time-dependent data as shown in FIG. 11B may provide valuable insights into the presence and kinetics of protease activity in clinical samples.

### Example 3. Analysis of a Clinical Sample of the Products of Neutrophil Elastase Proteolysis of a Substrate-Based Probe Using a Portable MALDI-TOFMS

The clinical sample 280-2 as previously described in Example 2 was analyzed using a portable MALDI-TOFMS developed by the Applicant. The portable MALDI-TOFMS system is described in commonly-owned U.S. Pat. No. 11,658,021 and U.S. patent application Ser. No. 18/133,441, which are incorporated by reference herein in each of their entireties.

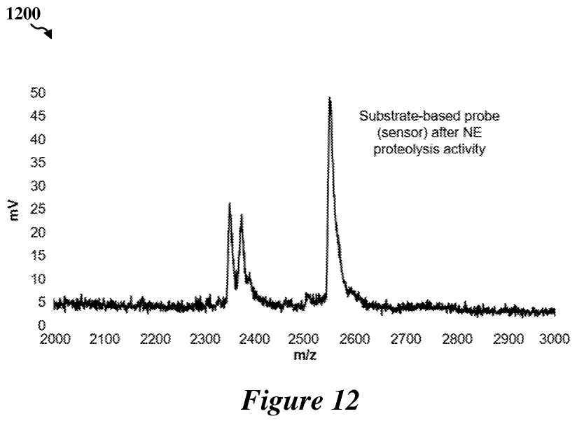

FIG. 12 shows a plot 1200 of MALDI-TOFMS mass spectra of an example substrate-based probe 700C under NE proteolysis of clinical sample 280-2 collected from a patient breathing using a ventilator, according to some implementations. As can be clearly seen, the new mass peaks of the substrate-based probe 700C were detected in clinical sample 280-2 indicative of NE proteolysis activity.

### Example 4. Detection of LRTI in Patients Breathing Through a Mechanical Ventilator by Capturing Neutrophil Elastase in Exhaled Air and Examining the Products of Neutrophil Elastase Proteolysis of a Substrate-Based Probe Using MALDI-TOFMS

Diagnostic assays were conducted to examine the sensitivity and specificity of an example substrate-based probe 700C (as shown in FIG. 7C) as previously described to detect lower tract respiratory infections (“LRTIs”) using captured neutrophil elastase in exhaled air of patients breathing through a ventilator. Non-volatile organic aerosolized particles (including neutrophil elastase in patients with LRTI) in the exhaled air aerosols of 13 patients diagnosed with LRTIs breathing through a ventilator (see FIG. 1B) were captured using sample capture element 101B. The patients were undergoing treatment at the Johns Hopkins Hospital Intensive Care Unit (“JHH ICU”). In addition, exhaled air aerosols were also captured from 15 patients diagnosed as not infected with LRTIs (“non-LRTI”) cases and from 19 healthy volunteers (“HV”). Breath samples were collected from healthy patients by breathing through a facial mask using a system as shown in FIG. 2.

As disclosed with reference to Example 2, the captured non-volatile organics were extracted, and one or more collected liquid clinical samples were reacted with substrate-based probe 700C. Human neutrophil elastase (“HNE”) proteolysis activity was examined using MALDI-TOFMS analysis of samples including substrate-based probes to detect LRTIs. As previously described, the detection of new substrate-based probe peaks at about 2357 m/z and about 2379 m/z may be considered as indicators of HNE proteolysis activity and subsequently and may be used to predict LRTIs. The exhaled air aerosols were extracted from the sample capture elements using the procedure disclosed in Example 2. Additionally, enzyme proteolysis of the extracted aerosol samples was initiated using the procedure disclosed in Example 2. The incubation period or reaction time of the NE proteolysis reaction sample solution was about 24 h. As previously described with reference to FIG. 9B, a shorter incubation period or reaction time may be used. The clinical samples were analyzed using MALDI-TOFMS to examine neutrophil elastase proteolysis activity of the substrate-probe.

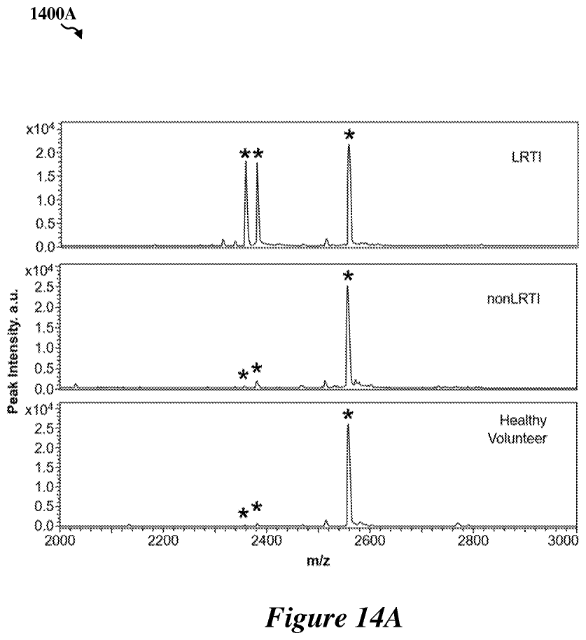

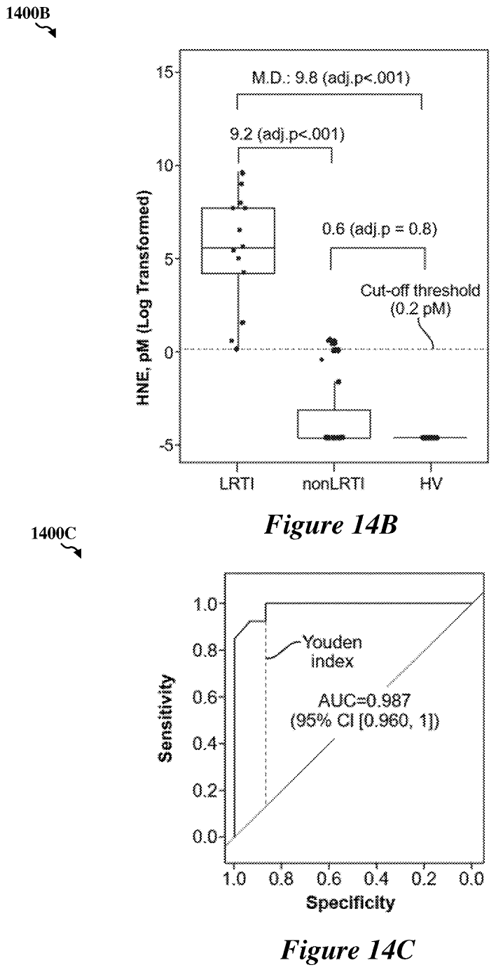

FIG. 14A shows MALDI-TOFMS mass spectra 1400A of clinical exhaled air samples collected from patients and volunteers after subjecting the samples to HNE proteolysis using substrate-based probe 700C, according to some implementations. As described above, the clinical samples were associated with a representative LRTI patient, a representative non-LRTI patient, and a representative healthy volunteer. As can be seen, the clinical samples associated with the non-LRTI patients and with healthy volunteers only show trace levels of new mass peaks (m/z: 2357 to 2379). In contrast, clinical samples associated with LRTI patients clearly show the presence of these new mass peaks associated with the substrate-based probe indicative of proteolysis activity and suggest upregulation of NE caused by an LRTI.

FIG. 14B shows a box and whisker plot 1400B for discriminating between LRTI patients and non-LRTI patients and healthy volunteers by determining HNE proteolysis activity of exhaled air samples using a substrate-based probe 700C, according to some implementations. The Y-axis in plot 1400B shows the concentration of HNE in the samples in picomole (“pM”) on a log-scale. The HNE concentration (y-axis) was estimated from calibration data obtained by analyzing samples of varying HNE concentrations using MALDI-TOFMS as previously disclosed with reference to FIG. 10. That is, the HNE concentration from each sample were calculated based on a calibration curve constructed using data collected from rmNE tests as disclosed in Example 1. The HNE concentration (or HNE levels) were natural-log-transformed for normalization, and prior to log-transformation, a constant of 0.01 was added to all zero values to prevent undefined logarithmic operations.

In the box and whisker plot 1400B, the boxes indicate quartiles, and the horizontal line within each box is indicative of the median HNE level in each case. The whiskers related to each “box” indicate the maximum and minimum of each range, with the lower and upper edges representing the first and third quartiles, respectively. Plot 1400B displays the distribution of HNE levels (or concentration) for each group. The distribution range of HNE measurements in the LRTI group or cohort was more extensive, indicating increased variability within this group as shown in FIG. 14B.

The pairwise differences in the mean or mean differences (“M.D.”) in HNE level between the groups LRTI, non-LRTI, and HV are also shown in plot 1400B. As can be seen, the pairwise difference in the mean HNE level of 9.2 pM between the LRTI and non-LRTI groups is statistically significant with an adjusted p-value of <0.001. Similarly, the pairwise difference in the mean HNE level of 9.8 pM between the LRTI and HV groups is also statistically significant with an adjusted p-value of <0.001. The mean differences and their corresponding adjusted p-values were derived by analysis of covariance (“ANCOVA”) with a Type I error significance level or false positive probability (α) set at 0.05 with Tukey's post hoc test. ANCOVA, implemented using the statistical programming language R, was used to assess the disparities in human neutrophil elastase (“HNE”) levels across the three groups LRTI, non-LRTI, and HV, with age and sex as covariates. Tukey's post hoc test generated adjusted p-values using Tukey's Honestly Significant Difference (“HSD”) method. As can be seen, the pairwise difference in the mean HNE level of 0.6 pM between the non-LRTI and HV groups was not statistically significant. Based on these results, analysis, and observations, a HNE cut-off threshold concentration of about 0.2 pM may be used to discriminate between LRTI patients and non-LRTI patients.

To further interrogate the above analysis, a logistic regression model was used to examine the relationship between confirmed LRTI infected groups or LRTI non-infected groups and their respective HNE concentrations. To calculate specificity, sensitivity, and a confusion matrix associated with the substrate-based probes for LRTO diagnosis, the designation of “LTRI” patients or “non-LTRI” patients was confirmed using other laboratory-based diagnostic methods. To assess the performance of the logistic regression model and determine its predictive accuracy, the Area Under the Curve (“AUC”) of the Receiver Operating Characteristic (“ROC”) curve was estimated. The ROC curve was constructed and area under the curve (AUC) was calculated between the LRTI and non-RTI groups after p-value adjustment. ROC curves may be used to assess the accuracy of a diagnostic test using a criterion variable such as HNE levels in a clinical sample which may be used to make a “yes” or “no” decision related to LRTI detection based on the value of this variable. The AUC may be viewed as a summary index of an ROC curve and may be indicative of the probability that a physician, or medical personnel, will correctly determine who is more likely to be infected with an LRTI using the diagnostic assay utilizing the substrate-based probes for LRTI disclosed herein. Alternately, the Youden Index (also referred to as Youden's J statistic) may be used as a summary measure of the ROC curve to measure the effectiveness of a diagnostic marker (HNE level in this case) and permits the selection of an optimal threshold value or cutoff point for the biomarker of interest to balance sensitivity and specificity. Statistical analysis was performed using R.

In diagnostic assays, the area under the ROC curve (AUC) may provide an overall measure of a biomarker/diagnostic assay's accuracy. The Youden index may be defined as the overall correct classification rate minus one at the optimal cut-off point. The index measures the effectiveness of a diagnostic marker and permits the selection of an optimal threshold value or cut-off point for the biomarker of interest. The Youden index may be defined as (sensitivity+specificity−1). The maximum value of the index (the point at which the slope of the ROC curve is zero) may be used as a criterion for selecting the optimum specificity cut-off point for a diagnostic test.

The ROC representative of HNE levels in the statistically significant groups may be constructed using the specificity (TN/TN+FP) and sensitivity (TP/TP+FN) values using the HNE levels as the predictive indicators of LRTI and using the actual indicators of LRTI confirmed using other assays, where TP, FN, TN, and FP indicate True Positive, False Negative, True Negative, and False Positive, respectively. An AUC value greater than at least about 60% may support the use of a biomarker or metric such as HNE levels to discriminate between LRTI and non-LRTI groups with high confidence.

FIG. 14C shows a ROC curve 1400C with AUC value for discriminating between LRTI and non-LTRI and HV groups by determining HNE proteolysis activity of exhaled air samples using a substrate-based probe 700C, according to some implementations. As shown in curve 1400C, an AUC of 98.7% at a confidence interval (“CI”) of 95% suggests that a LTRI diagnosis made using the substrate-based probe 700C to examine HNE proteolysis activity using MALDI-TOFMS may be used to discriminate between LRTI and non-LRTI groups with high confidence.

Additionally, the cut-off point for the Youden index occurs at a specificity value of about 0.867 and a sensitivity value of about 1 as shown in FIG. 14C. The threshold that provides the best specificity and sensitivity in a ROC curve is often defined using Youden's index. Without being bound by any particular theory, the cut-off point for the Youden index represented as a maximum value of the ROC curve may be associated with at least 0.85 (specificity value). As previously discussed, the cut-off threshold HNE concentration may be about 0.2 pM associated with diagnostic assays for LRTIs using the example substrate-based probe 700C as disclosed herein. Accordingly, an in vitro assay and a non-invasive approach for LRTI diagnosis in critical care may include capturing human breath aerosols as clinical samples and analyzing HNE proteolysis activity of the clinical samples using specific substrate-based sensors. The disclosed substrate-based sensors are sensitive, which is critical to detect low concentrations of proteins such as human neutrophil elastase in breath.

In some implementations, a method for predicting a lower respiratory tract infection (LRTI) using captured aerosol particles exhaled air samples may include generating calibration data, for example, as previously discussed with reference to FIG. 10, that correlates the concentration of human neutrophil elastase (HNE) to the peak intensity of one or more characteristic mass peaks associated with MALDI-TOFMS analysis of HNE proteolysis samples, wherein HNE proteolysis is initiated using a substrate-based probe, subjecting a test sample including aerosol particles captured from a patient suspected of being infected with a LRTI to HNE proteolysis using the substrate-based probe, analyzing the test sample after HNE proteolysis using MALDI-TOFMS, estimating the HNE concentration in the test sample using the calibration data, and predicting the presence of LRTI if the HNE concentration in the test sample is greater than or equal to a cut-off threshold HNE concentration. In some implementations, the substrate-based probe may include any of the substrate-based probes previously disclosed herein.

In some implementations, the cut-off threshold HNE concentration may be about 0.2 picomol (“pM”).

In some implementations, the method for predicting a lower respiratory tract infection (LRTI) using captured aerosol particles exhaled air samples may further include generating a confusion matrix related to identifying LRTI infected patients and non-LRTI infected patients by examining HNE proteolysis activity of captured aerosol particles in their respective exhaled air samples using the substrate-based probe.

In some implementations, the cut-off threshold HNE concentration may be a variable selected depending on a predetermined sensitivity value associated with the confusion matrix. For example, during screening tests for LRTI, the cut-off threshold HNE concentration may be increased to ensure that clinical samples associated with a HNE concentration greater than the cut-off threshold HNE concentration would be 100% positive for a LTRI infection. An example confusion matrix is shown in Table 1 below:

TABLE 1
Confusion matrix template for predicting LRTI using a substrate-
based probe for HNE proteolysis of samples including exhaled
breath aerosols collected during clinical trials.

| | | Predicted | |
|---|---|---|---|
| | | Negative (0) | Positive (1) |
| Actual | Negative (0) | TN | FP (Type I error) |
| | Positive (1) | FN (Type II error) | TP |
| | Total | | |

TN = True Negative, TP = True Positive, FN = False Negative, FP = False Positive.

As previously described, specificity (TN/TN+FP) and sensitivity (TP/TP+FN) values may be calculated. In some implementations, an example substrate-based probe may include any of the substrate-based probes previously described herein.

In some implementations, exhaled air from patients breathing using a ventilator may be collected using example exhaled air aerosol collection system 100B (referring to FIG. 1B) including a packed bed column. In some other implementations, exhaled air from patients may be collected using example exhaled breath capture system 200 (referring to FIG. 2) including a packed bed column.

In some implementations, the substrate-based probe may include the probe 700C (with reference to FIG. 7C).

### Example 5. Correlation Between HNE Activity and Protein Levels in Exhaled Air Samples from Patients Breathing Using a Mechanical Ventilator

To investigate the potential correlation between observed HNE activity and total HNE concentration in the exhaled air samples captured using the methods and systems previously disclosed herein, bottom-up proteomics analysis was conducted on exhaled air samples collected from intubated patients using the methods previously disclosed herein.

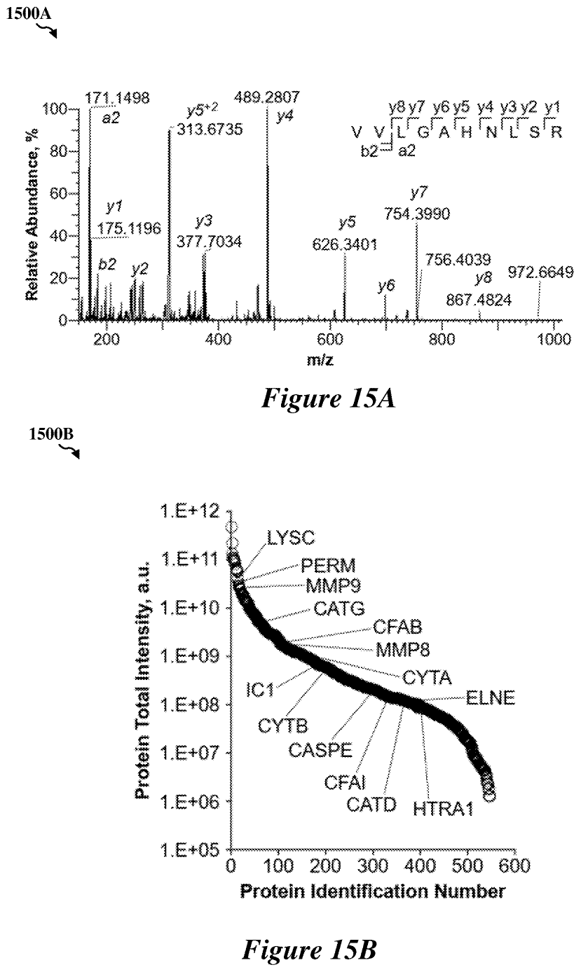

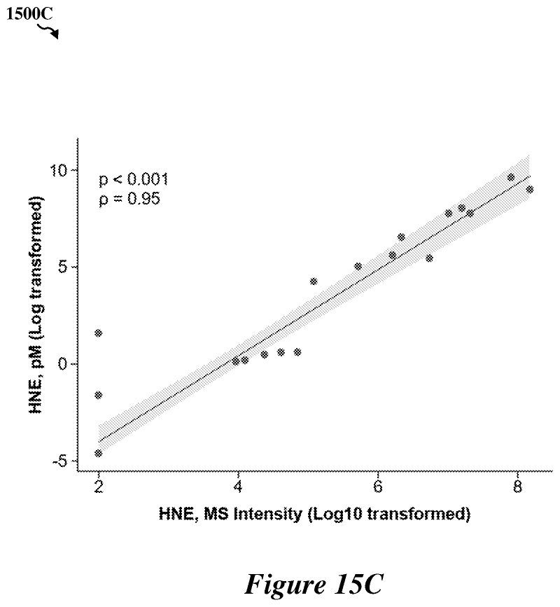

FIG. 15A shows a representative ion fragmentation map 1500A of a HNE peptide using bottom-up proteomics, according to some implementations. FIG. 15B shows a protein total intensity profile 1500B of proteases and anti-proteases in captured exhaled air samples, according to some implementations. As can be seen, the peptide VVLGAHNLSR characteristic of HNE was identified, in addition to the other proteases and anti-proteases, including MMP8, MMP9, and cathepsin G. Among the detected proteases, HNE (shown as ELNE in FIG. 15B) had the weakest protein intensity, suggesting a lower protein concentration relative to the other proteins identified. During mass spectrometry, samples were introduced to an LTQ orbitrap mass spectrometer (Thermo Fisher Scientific) with electrospray ionization (ESI) capacity via direct infusion as a flow rate of 6 μL/minute. Precursor profiles were acquired in the positive ion mode with the resolution of 60,000. Ion fragmentation profiles were acquired from the same mass spectrometer with 35% collision-induced dissociation (CID) energy with an isolation window of 1.5 m/z. The raw mass spectrometry files were processed and deconvoluted profiles were acquired using FreeStyle Software (Thermo Fisher Scientific). Deconvoluted profiles and ion fragmentation patterns were interpreted manually in house.

FIG. 15C shows a correlation plot 1500C between HNE concentration and mass spectra intensity of characteristic mass peaks associated with HNE, according to some implementations. A highly positive association between the quantified HNE concentration and intensity of mass spectra peaks associated with HNE in the breath samples can be seen as validated by a Spearman's Rho of 0.95 at a p-value of <0.001.

### Example 6. Sensitivity and Specificity of the Example NE Substrate-Based Probe 700C to Proteolysis of Human Proteases

The sensitivity and specificity of the example NE substrate-based probe 700C to enzyme reaction cleavage products as a function of time during proteolysis of 15 pM recombinant human neutrophil elastase (“rhNE,” Catalog #9167-SE, R&D Systems, Minneapolis, MN), 15 pM human cathepsin G (“CTSG, Catolog #ab91122, Abcam), and 15 μM human proteinase 3 (“PR3,” Product #16-14-161820, Athens Research & Technology) were examined.

To this end, example sensor 700C was incubated with a blank control sample (“control”) consisting of mass spectrometer (MS)-grade water and with the different proteases, including 15 μM rhNE, 15 μM human cathepsin G, and 15 μM human proteinase. Mass spectra of samples collected after incubation period of 0 min, 10 min, 30 min, 60 min, 120 min, and 180 min were examined to determine the ratio of the peak intensity of cleaved product to that of the intact substrate (that is, the sensor) over time.

For MALDI-TOFMS analysis of sample, about 1 μL of each reaction (proteolysis) sample solution was added to a Bruker MALDI plate spot plate (MSP 96 target ground steel). The MALDI plate was transferred to a heating block set to 60° C. until the spots were dry. The MALDI plate was removed from the heating block and 1.0 μL of α-Cyano-4-hydroxycinnamic acid (“CHCA”) MALDI matrix (9 mg/mL in 70% acetonitrile) was added to the sample spot. The MALDI plate was then transferred back to the heating block set to 60° C. until spots were dry. The samples were characterized using a Bruker AutoFlex MALDI-TOF instrument set in positive ion linear mode.

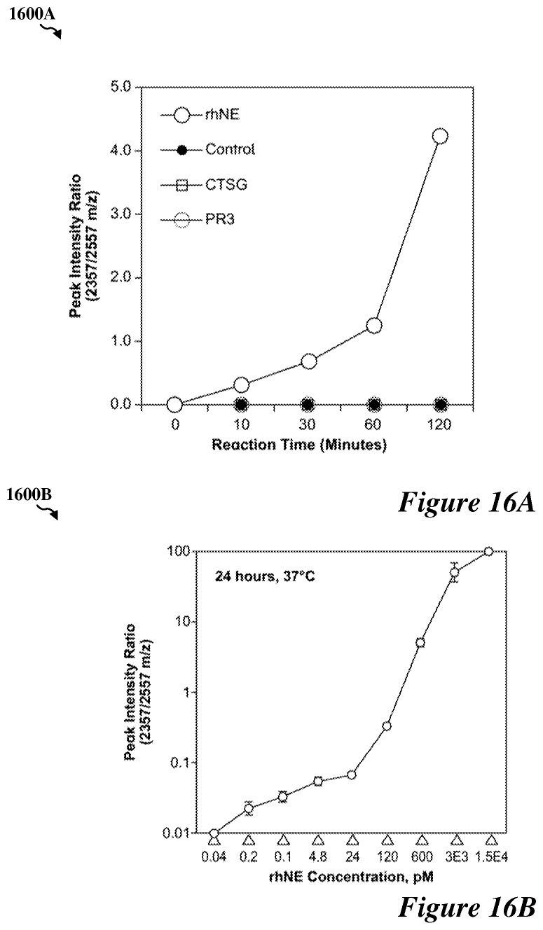

FIG. 16A shows a plot 1600A illustrating the specificity of an example substrate-based sensor to proteolysis of rhNE, according to some implementations. FIG. 16A shows the ratio of the MALDI-TOFMS mass spectral peak intensity associated with new mass spectra peak (2357 m/z) corresponding to the cleaved product resulting from proteolysis of each of the human proteases using the example sensor to the intensity associated with the intact sensor (at about 2557 m/z) as a function of reaction time. As can be seen, with respect to rhNE, the ratio of the peak intensity associated with the cleavage product mass peak at 2357 m/z to that of the intact sensor (2557 m/z) increased with an increase in incubation period indicative of strong correlation between the mass intensity ratio as described above, with NE proteolysis activity.

In contrast to proteolysis of rhNE, during analysis of samples related to proteolysis of human cathepsin G, and human proteinase 3, the mass peak associated with the cleavage product resulting from proteolysis using the example sensor was not detected, suggesting that the example sensor is specific only to proteolysis of rhNE. Without being bound by any particular theory, the products of rhNE proteolysis using the example substrate-based probe 700C may be detected after an incubation period of between about 5 min. and about 10 min. and at between about 20° C. and about 70° C., to realize rapid detection of lower respiratory tract infections using the example substrate-based probes described herein. In some instances, the incubation period may decrease with increasing incubation temperature. Accordingly, the incubation period and temperature may be tuned to quickly generate the cleavage product resulting from proteolysis of rhNE using an example substrate-based sensor as described herein.

FIG. 16B shows a plot 1600B illustrating the sensitivity of an example substrate-based sensor to proteolysis of rhNE, according to some implementations. Various concentrations of rhNE ranging from 0.04 pM (pico molar) to 1.5E4 pM were prepared and incubated with the example sensor 700C for 24 h at 37° C. The resultant proteolysis samples were prepared for MALDI-TOFMS analysis, as previously described herein. As can be seen, the ratio of the MALDI-TOFMS mass spectral peak intensity associated with new mass spectra peak (2357 m/z) corresponding to the cleaved product resulting from proteolysis of rhNE using the example sensor to the intensity associated with the intact sensor (at about 2557 m/z) as a function rhNE initial concentration, increased with reNE initial concentration. The cleavage product peak (at 2357 m/z) was observed in samples even at the low rhNE concentration of 0.04 pM. This detection capability, even at extremely low concentrations of rhNE, demonstrated the sensitivity of the example customized HNE substrate sensor. Additionally, the example calibration curve shown in FIG. 16B, may be used to estimate the hNE concentration in various samples, including clinical breath samples.

Accordingly, the substrate-based sensors described herein show significant sensitivity and specificity to rhNE, which is critical for detecting ultra-low protease levels in breath samples of patients with lower respiratory tract infections. Breath samples may include parts-per-billion (ppb) levels of protein materials. Additionally, the response of the sensors may be correlated to rhNE concentrations using MALDI-TOFMS analysis for prediction of lower respiratory tract infection in patients, with high specificity and sensitivity.

### Example 7. Detection of RTI in Patients Breathing Through a Mechanical Ventilator by Capturing Proteases in Exhaled Air and Examining the Products of Proteolysis of a Multiplex Substrate-Based Probe Assay Using MALDI-TOFMS

Non-volatile organic aerosolized particles (including proteases in patients with RTI) in exhaled air aerosols of 132 patients breathing through a ventilator (see FIG. 1B) were captured using sample capture element 101B. The patients were undergoing treatment at four intensive care units (ICUs) at Johns Hopkins Hospital (“JHH”). Breath samples were collected along with detailed patient information including age, gender, race, ethnicity, primary diagnosis, medication, sample collection time, microorganism identification information, white blood cell test results, body temperature, fraction of inspired oxygen (FiO₂) levels, and pulmonary radiography data. To establish a baseline, lower respiratory tract infection (“LRTI”) was diagnosed by physicians using a multiplex substrate-based probe assay, clinical criteria was confirmed through positive cultures of tract samples, including sputum, endotracheal tube samples (“ET”), or bronchoalveolar lavage (“BAL”), in the clinical laboratory. Of the 132 patients, 49 LRTI cases and 83 non-LRTI cases were identified.

The multiplexing assay associated with exhaled breath analysis including three substrate-based probes for detecting proteolysis activity as summarized in Table 2 below:

TABLE 2
Summary of three substrate-based probes for detecting
proteolysis activity using a multiplexing assay.

| | Substrate | | Second order |
|---|---|---|---|
| | Sensor | Protease | rate constant |
| Sensor ID | Composition | Target | k_{cal}/Kₘ (M⁻¹s⁻¹) |
| S1: | PEG36- Nle(OBzl)- | HNE | 4.8 × 10⁷ |
| | Met(O)2-Oic- | | |
| | Abu -ACC | | |
| S2: | PEG40- Pro-Leu- | MMP8, MMP9 | 1.1 × 10⁶, 3.0 × 10⁶ |
| | Gly-Leu-Lys-Ala- | | |
| | Arg-Arg | | |
| S3: | PEG45- Lys-Pro- | MMP8, MMP9 | 2.0 × 10⁶, 3.0 × 10⁶ |
| | Leu-Gly-Leu-Lys- | | |
| | Ala-Arg | | |

As can be seen, the first probe (“S1) targeted HNE proteolysis and the second probe (“S2”) and third probe (“S3”) targeted proteolysis of MMPs captured from exhaled breath. In particular, substrate-based probe S2 targeted MMP8 proteolysis and substrate-based probe S3 targeted MMP9 proteolysis. Details associated with these probes were previously described herein. The rate constants associated with proteolysis activity using each of the respective probes are also shown in Table 2.

Proteases were eluted from the sample capture element 101B using 100 μL of 70% acetonitrile (v/v in water). The extracted collected liquid samples were then added to 100 μL of a substrate-based probe solution including the three substrate-based probes shown in Table 2. The solution included reaction buffer (50 mM Tris, 1 M NaCl, 0.05% (w/v) Brij-35, pH 7.5) and about 200 μM of each of the substrate-based probes. After incubating for 24 hours at 37° C., about 1 μL of the sample was placed onto a MALDI plate and dried. Subsequently, about 1 μL of α-cyano-4-hydroxycinnamic acid MALDI matrix (9 mg/mL in 70% acetonitrile) was added onto the sample and dried.

MALDI-TOF MS profiles were acquired using a Bruker Daltonics microflex LRF mass spectrometer (Billerica, MA) in positive linear mode. Mass spectra were obtained from 500 profiles between a mass range of about 1500 m/z and about 3000 m/z. The ion intensity values of the mass peaks of interest were extracted from the raw files generated directly from the operation software on the Bruker instrument. Quantitative measurement of the protease activity was done by calculating the ratio of cleavage products to intact sensors and correlating with a calibration curve constructed from a limits of detection (“LOD”) examination associated with each of the substrate-based probes (also referred to herein as sensors).

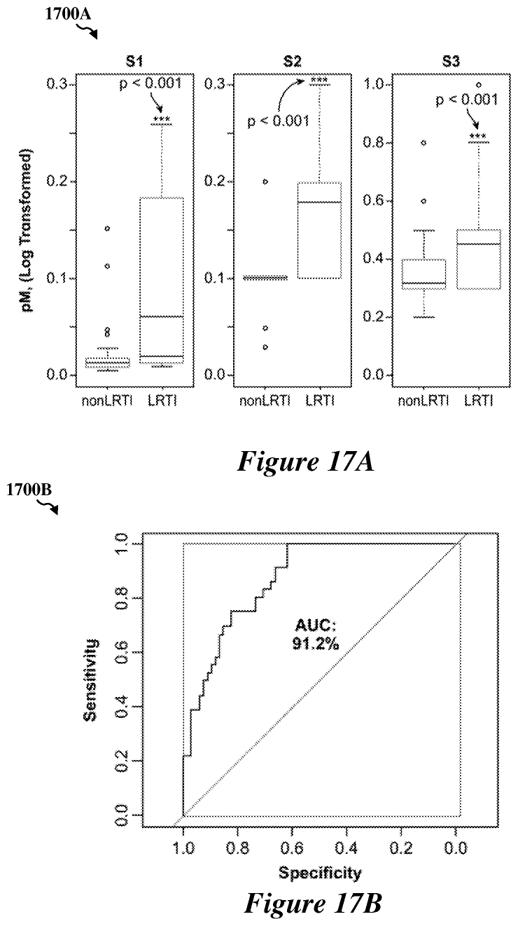

FIG. 17A shows box and whisker plots 1700A for discriminating between LRTI patients and non-LRTI patients by determining proteolysis activity of exhaled air samples using various substrate-based probes, according to some implementations. The Y-axis in plot 1700A shows the concentration of proteases HNE, MMP8, and MMP9 in the collected liquid samples in picomole (“pM”) on a log-scale associated with each of the respective probes S1-S3 shown in Table 2. As previously described herein, in the box and whisker plots 1700A, the boxes indicate quartiles, and the horizontal line within each box is indicative of the median protease level in each case. The whiskers related to each “box” indicate the maximum and minimum of each range, with the lower and upper edges representing the first and third quartiles, respectively. Plot 1700A displays the distribution of the concentration of each of the proteases for each group. The distribution range of protease measurements in the LRTI group or cohort was more extensive, indicating increased variability within this group as shown in FIG. 17A. As can be seen, a significantly elevated level of each of the three proteases were observed in the samples collected from LRTI patients (p-value<0.001). Box and whisker plots for discriminating between LRTI patients and non-LRTI patients using multiplexed assay including all three probes (not shown for convenience) also showed a significantly higher level of protease concentrations in the samples collected from LRTI patients.

Additionally, a logistic regression model was used to examine the relationship between confirmed LRTI infected groups or LRTI non-infected groups and protease concentrations in their respective collected liquid samples. To calculate specificity, sensitivity, and a confusion matrix associated with the substrate-based probes for LRTI detection, the designation of “LTRI” patients or “non-LTRI” patients was confirmed using other laboratory-based diagnostic clinical methods, as previously described herein. To assess the performance of the logistic regression model and determine its predictive accuracy, the Area Under the Curve (“AUC”) of the Receiver Operating Characteristic (“ROC”) curve was estimated. The ROC curve was constructed and area under the curve (AUC) was calculated between the LRTI and non-RTI groups after p-value adjustment. ROC curves may be used to assess the accuracy of a diagnostic test using a criterion variable such as protease levels in a clinical sample which may be used to make a “yes” or “no” decision related to LRTI detection based on the value of this variable. The AUC may be viewed as a summary index of an ROC curve and may be indicative of the probability that a physician, or medical personnel, will correctly determine who is more likely to be infected with an LRTI using the diagnostic assay utilizing the substrate-based probes for LRTI disclosed herein.

FIG. 17B shows a ROC curve 1700B with AUC value for discriminating between LRTI and non-LTRI by determining protease proteolysis activity of exhaled air samples using a multiplexed assay including three probes, according to some implementations. As shown in curve 1700B, an AUC of 91.2% at a confidence interval (“CI”) of 95% suggests that a LTRI diagnosis made using multiplexed assay including all three probes and MALDI-TOFMS may be used to discriminate between LRTI and non-LRTI groups with high confidence.

### Example 8. Analysis of Exhaled Breath from Patients Breathing Through a Mechanical Ventilator Collected Before Clinical Detection of Symptoms by Capturing Proteases in Exhaled Air and Examining the Products of Proteolysis of a HNE Substrate-Based Probe Using MALDI-TOFMS

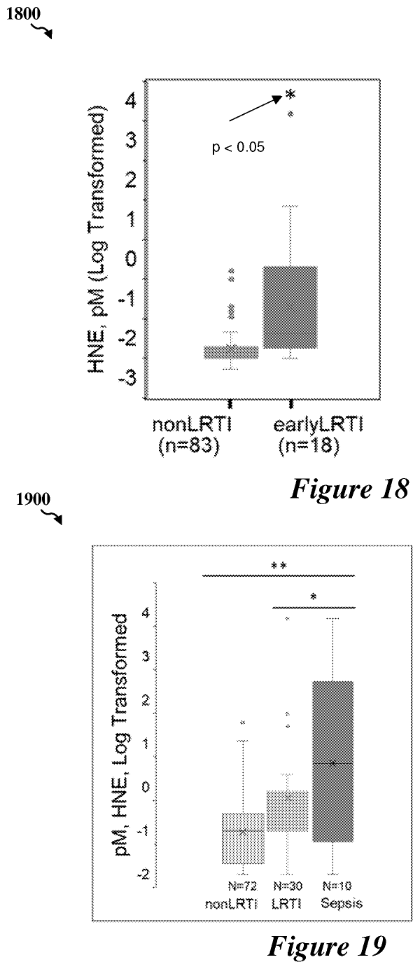

Exhaled breath samples from mechanically ventilated patients at JHH were collected several days before the patients showed clinical symptoms and were diagnosed with LRTI. Proteases in exhaled breath were captured using sample capture elements and were eluted as collected liquid samples as previously described with reference to Example 7. The HNE substrate-based probe (“S1”) listed in Table 2 was used as a proteolysis probe. FIG. 18 shows a box and whisker plot 1800 for early detection of LRTI by determining proteolysis activity of exhaled air samples using a HNE proteolysis substrate-based probe. As can be seen, HNE activity was significantly higher in early LRTI cases, suggesting that the substrate-based probe described herein may be used to diagnose early LRTI cases before the clinical diagnosis based on symptoms.

### Example 9. Analysis of Exhaled Breath from Sepsis Patients Breathing Through a Mechanical Ventilator by Capturing Proteases in Exhaled Air and Examining the Products of Proteolysis of a HNE Substrate-Based Probe Using MALDI-TOFMS

Lower respiratory tract infection (“LRTI”) is a leading precursor to sepsis in mechanically ventilated patients, necessitating immediate medical intervention when sepsis occurs. Sepsis is diagnosed when two or more systemic inflammatory response syndrome (“SIRS”) criteria are met in conjunction with an active infection.

Exhaled breath samples from mechanically ventilated patients at JHH were collected to detect sepsis cases patients breathing using a mechanical ventilator. Proteases in exhaled breath were captured using sample capture elements and were eluted as collected liquid samples as previously described with reference to Example 7. The HNE substrate-based probe (“S1”) listed in Table 2 was used as a proteolysis probe. FIG. 19 shows a box and whisker plot 1900 for detecting sepsis cases by determining proteolysis activity of exhaled air samples using a HNE proteolysis substrate-based probe. As can be seen, HNE activity was significantly higher in sepsis cases, suggesting that the substrate-based probes described herein may be used to diagnose sepsis cases. Additionally, these results indicate a statistically significant difference in HNE levels among the three groups, with the sepsis group showing markedly higher HNE activity compared to the non-LRTI and LRTI groups. As such, HNE may serve as a potential biomarker for detecting sepsis cases in this patient population.

### Example 10. Detection of Causative Pathogens Using qPCR in Collected Liquid Samples Including Aerosolized Non-Volatile Particles Including One or More Proteases Present in Exhaled Air

As previously described with reference to Example 7, of the 132 intubated patients examined for LRTI by analyzing the proteolysis activity of one or more proteases in exhaled air using a plurality of substrate-based probes, 49 positive LRTI cases and 83 non-LRTI cases were identified.

The collected liquid samples including aerosolized non-volatile particles including one or more proteases present in exhaled air associated with the 49 positive cases were then analyzed using a qPCR assay to identify the causative pathogen associated with the LRTI in each case.

The qPCR assay was conducted using an Applied Biosystems ABI 7500 Fast Dx Real-Time PCR System on 96 well PCR plates. Each PCR reaction mixture included TaqPath™ DuraPlex™ 1-Step RT-qPCR Master Mix and target specific TaqMan™ Microbe Detection Assay (Thermo Fisher Scientific), as listed in Table 3 below:

TABLE 3
Example composition of a reaction mixture
associated with qPCR analysis.

| | Volume per |
|---|---|
| Component | reaction |
| TaqPath ™ DuraPlex ™ 1-Step | 5 μL |
| RT-qPCR Master Mix (4X) | |
| TaqMan ™ Assay (20X) | 1 μL |
| JHH Exhaled Breath Sample | 4 μL |
| Nuclease-Free Water | 10 |
| Total Volume | 20 μL |

The target pathogen included Acinetobacter baumannii, Klebsiella pneumoniae, Pseudomonas aeruginosa, Staphylococcus aureus, Haemophilus influenza, Streptococcus pneumoniae, Escherichia coli, and Enterobacter cloacae. The pathogens were selected based on clinical culture test results, which served as the reference standard. Pathogen identification using the qPCR assay was then compared with the reference standard.

The testing thermal conditions associated with the qPCR assay are summarized in Table 4 below:

TABLE 4
Thermal conditions associated with qPCR analysis.

| Steps | T, ° C. | Time, s | Cycles |
|---|---|---|---|
| Reverse | 53 | 600 | 1 |
| Transcription | | | |
| Polymerase | 95 | 120 | 1 |
| activation | | | |
| Denature | 95 | 3 | 40 |
| Anneal/extend | 60 | 30 | |

Table 5 summarizes pathogen identification using the qPCR assay associated with the 49 LRTI positive samples and provides a comparison with the respective reference standards:

TABLE 5
Results of a qPCR assay associated with 49 LRTI positive samples and
provides a comparison with the respective reference standards.

| Collected liquid samples | | | | | |
|---|---|---|---|---|---|
| including non-volatile | | | | | |
| particles including proteases | | | | | |
| | Proteolysis | | | | |
| | using substrate- | | Clinical | | |
| Testing | based probes | | Diagnosis Method | | |
| ID | (LRTI) | qPCR | Specimen | Method | Results |
| 13 | Positive | E cloacae | Sputum, ETA | Culture | E cloacae |
| 14 | Positive | P aeruginosa | Sputum, ETA | Culture | P aeruginosa |
| 15 | Positive | E cloacae | BAL, Blood | Culture | E cloacae |
| 17 | Positive | A baumannii | Sputum | Culture | A baumannii |
| 20 | Positive | K pneumoniae | Sputum | Culture | K pneumoniae |
| 21 | Positive | P aeruginosa | Sputum | Culture | P aeruginosa |
| 25 | Positive | E cloacae | BAL, Blood | Culture | E cloacae |
| 26 | Positive | S aureus | Sputum, ETA | Culture | S aureus |
| 27 | Positive | E cloacae | ETA | Culture | E cloacae |
| 31 | Positive | E cloacae | ETA | Culture | E cloacae |
| 33 | Positive | P aeruginosa | Sputum | Culture | P aeruginosa |
| 37 | Positive | P aeruginosa | Sputum | Culture | P aeruginosa |
| 38 | Positive | P aeruginosa | Sputum | Culture | P aeruginosa |
| 39 | Positive | P aeruginosa | Sputum | Culture | P aeruginosa |
| 53 | Positive | S aureus | ETA | Culture | S aureus |
| 57 | Positive | P aeruginosa | Sputum | Culture | P aeruginosa |
| 63 | Positive | E coli | BAL, Blood | Culture | E coli |
| 65 | Positive | E coli | BAL, Blood | Culture | E coli |
| 69 | Positive | E coli | ETA | Culture | E coli |
| 79 | Positive | E coli | BAL, Blood | Culture | E coli |
| 110 | Positive | P aeruginosa | BAL | Culture | P aeruginosa |
| 117 | Positive | S aureus | BAL, Blood | Culture | S aureus |
| 118 | Positive | S aureus | ETA | Culture | S aureus |
| 123 | Positive | S aureus | ETA | Culture | S aureus |
| 124 | Positive | S aureus | Sputum | Culture | S aureus |
| 127 | Positive | P aeruginosa | Sputum | Culture | P aeruginosa |
| 136 | Positive | P aeruginosa | Sputum | Culture | P aeruginosa |
| 137 | Positive | S aureus | Sputum | Culture | S aureus |

Referring to Table 5, the qPCR assay detected the presence of pathogens in 28 samples. Among these 28 detected samples, the qPCR assay correctly identified the respective pathogen in each case, resulting in a pathogen identification accuracy of 100%. As previously described with reference to Example 7, proteolysis activity of proteases HNE, MMP8, and MMP9 in the collected liquid samples (including the 28 samples) was used to predict LRTI in patients. Positive LRTI was predicted when the protease concentration in the tested samples exceeded a predefined threshold value. As such, proteolysis activity may be used to differentiate between infection and colonization by measuring host response markers in the collected liquid samples obtained using the methods described herein for capturing aerosolized non-volatile particles in exhaled air.

When considered together with the qPCR assay, this sequential analysis method may not only confirm the presence of an active LRTI, but may also provide pathogen-specific information, and offers a comprehensive solution for managing LRTI cases. Accordingly, the sequential analysis method described herein may be quick and characterized by high sensitivity and specificity compared to conventional diagnostic methods commonly used in hospitals, including bronchoalveolar lavage (“BAL”) culture, which relies on invasive specimen collection and often requires an analysis time of several days. In contrast, the sequential analysis method described above offers an analytical result by leveraging analysis of non-invasive exhaled air samples (as collected liquid samples) and integrating rapid host response detection with pathogen identification.

Additional details related to example sample capture elements are disclosed in commonly owned International Appl. No. PCT/US2020/048035, which is incorporated by reference herein in its entirety.

As used herein, a phrase referring to “at least one of” or “one or more of” a list of items refers to any combination of those items, including single members. For example, “at least one of: a, b, or c” is intended to cover the possibilities of: a only, b only, c only, a combination of a and b, a combination of a and c, a combination of b and c, and a combination of a and b and c. Unless otherwise specified in this disclosure, for construing the scope of the term “about” or “approximately,” the error bounds associated with the values (dimensions, operating conditions etc.) disclosed is ±10% of the values indicated in this disclosure. The error bounds associated with the values disclosed as percentages is ±1% of the percentages indicated. The word “substantially” used before a specific word includes the meanings “considerable in extent to that which is specified,” and “largely but not wholly that which is specified.”

Various modifications to the implementations described in this disclosure may be readily apparent to persons having ordinary skill in the art, and the generic principles defined herein may be applied to other implementations without departing from the spirit or scope of this disclosure. Thus, the claims are not intended to be limited to the implementations shown herein but are to be accorded the widest scope consistent with this disclosure, the principles and the novel features disclosed herein.

Additionally, various features that are described in this specification in the context of separate implementations also can be implemented in combination in a single implementation. Conversely, various features that are described in the context of a single implementation also can be implemented in multiple implementations separately or in any suitable subcombination. As such, although features may be described above in combination with one another, and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flowchart or flow diagram. However, other operations that are not depicted can be incorporated in the example processes that are schematically illustrated. For example, one or more additional operations can be performed before, after, simultaneously, or between any of the illustrated operations. In some circumstances, multitasking and parallel processing may be advantageous. Moreover, the separation of various system components in the implementations described above should not be understood as requiring such separation in all implementations, and it should be understood that the described components and systems can generally be integrated together in a single product or packaged into multiple products.

### REFERENCES

Hunt, J., “Exhaled breath condensate: An evolving tool for noninvasive evaluation of lung disease,” J. Allergy Clin. Immunol. 2002; 110:28-34.
J. Brennan McNeil, Ciara M. Shaver, V. Eric Kerchberger, Derek W. Russell, Brandon S. Grove, Melissa A. Warren, Nancy E. Wickersham, Lorraine B. Ware, W. Hayes McDonald, and Julie A. Bastarache, “Novel Method for Noninvasive Sampling of the Distal Airspace in Acute Respiratory Distress Syndrome,” American J. Respiratory and Critical Care Medicine 197(8), Apr. 15, 2018.
Benjamin Patterson, Carl Morrow, Vinayak Singh, Atica Moosa, Melitta Gqada, Jeremy Woodward, Valerie Mizrahi, Wayne Bryden, Charles Call, Shwetak Patel, Digby Warner, Robin Wood, “Detection of Mycobacterium tuberculosis bacilli in bio-aerosols from untreated TB patients,” Gates Open Research 2018, 1:11.
Joerg Reifart, Christoph Liebetrau, Christian Troidl, Katharina Madlener and Andreas Rolf, “Noninvasive sampling of the distal airspace via HME-filter fluid is not useful to detect SARS-CoV-2 in intubated patients,” Crit. Care (2021) 25:126.
Natacha Jugniot, Pierre Voisin, Abderrazzak Bentaher, and Philippe Mellet, “Neutrophil Elastase Activity Imaging: Recent Approaches in the Design and Applications of Activity-Based Probes and Substrate-Based Probes,” Contrast Media & Molecular Imaging, vol. 2019, Article ID 7417192.
Jin-Ha Choi, “Proteolytic Biosensors with Functional Nanomaterials: Current Approaches and Future Challenges,” Biosensors, 2023, 13, 171.
Paulina Kasperkiewicz, Marcin Poreba, Scott J. Snipas, Heather Parker, Christine C. Winterbourn, Guy S. Salvesen, and Marcin Drag, “Design of ultrasensitive probes for human neutrophil elastase through hybrid combinatorial substrate library profiling,” PNAS, 111 (7), February 2014, p 2518.

What is claimed is:

1. An exhaled breath capture module configured to capture aerosolized non-volatile organic particles in exhaled breath, the module including:
an inlet duct configured to receive a mouthpiece removably insertable into the inlet duct;
an outlet port disposed opposite to the inlet duct, wherein an outlet tubing including an inlet end and an outlet end is removably insertable into the outlet port and extend into the inlet duct defining an annular region between the outlet tubing and the inlet duct;
an exhaled breath capture element including a packed bed column configured to capture the aerosolized non-volatile organic particles in exhaled breath, wherein the exhaled breath capture element is removably insertable into the inlet end of the outlet tubing defining a gap of predetermined length between an outlet of the mouthpiece and the exhaled breath capture element;
a reservoir region disposed in fluid communication with the inlet duct and the annular region and configured to collect saliva particles or breath condensate particles in the exhaled breath that drop through the gap, wherein the outlet end of outlet tubing is coupled to a pump configured to draw the exhaled breath through the packed bed column; and
a vent disposed in fluid communication with the reservoir region, wherein the vent is configured to prevent pressure build-up in the exhaled breath capture module.

2. The exhaled breath capture module of claim 1, wherein the vent is disposed in an orthogonal orientation relative to a longitudinal axis (A-A′) associated with the inlet duct.

3. The exhaled breath capture module of claim 1, further including a HEPA filter coupled to the vent.

4. The exhaled breath capture module of claim 1, wherein the packed bed column is activated by one or more of:
washing the packed bed column with one or more of isopropyl alcohol or methanol; or
washing with water, wherein after the packed bed column is activated, each of an inlet and outlet of the sample capture element is capped.

5. The exhaled breath capture module of claim 1, wherein the aerosolized non-volatile organic particles include one or more of metabolite biomarkers, lipid biomarkers, proteomic biomarkers, bacteria particles, or virus particles characteristic of one or more respiratory diseases.

6. The exhaled breath capture module of claim 1, wherein the packed bed column includes solid particles including one or more of resins, cellulose, silica, agarose, or hydrated Fe₃O₄ particles.

7. The exhaled breath capture module of claim 1, wherein the packed bed column includes resin beads having one or more of C18 functional groups on the surface, cellulose beads having sulfate ester functional groups on the surface, or a mixture thereof.

8. The exhaled breath capture module of claim 7, wherein a diameter of the resin beads is at least about 20 μm.

9. The exhaled breath capture module of claim 1, wherein the packed bed column includes:
a first packed bed including silica gel beads; and
a second packed bed including resin beads functionalized with C18 groups, wherein the second packed bed is disposed downstream of the first packed bed.

10. The exhaled breath capture module of claim 9, wherein the silica gel beads have an average diameter of between about 20 μm and about 500 μm.

11. The exhaled breath capture module of claim 9, wherein the resins beads functionalized with C18 groups have an average diameter of between about 20 μm and about 500 μm.

12. The exhaled breath capture module of claim 1, wherein the pump is configured to draw exhaled breath through the exhaled breath capture element at a flow rate of between about 0.5 L/min and about 10 L/min.

13. A system for diagnosing a respiratory disease, the system including:
the exhaled breath capture module of claim 1;
an extraction means configured to produce one or more liquid samples by extracting the aerosolized non-volatile organic particles from the packed bed column using a solvent; and
an analytical device configured to analyze the particles in the one or more liquid samples.

14. The system of claim 13, wherein the analytical device includes one or more of PCR, ELISA, rt-PCR, mass spectrometer (MS), MALDI-MS, ESI-MS, MALDI-TOFMS, or LC-MS/MS.

15. The system of claim 13, wherein the solvent includes one or more of acetonitrile (ACN), methanol, trifluoro acetic acid (TFA), or isopropanol (IPA), the remaining being water.

16. The system of claim 13, wherein the solvent includes between about 50 vol.-% and about 70 vol.-% acetonitrile in water, between about 50 vol.-% and about 70 vol.-% isopropanol in water, and between about 0.05 vol.-% TFA in water.

17. A method for analyzing aerosolized non-volatile particles including proteases in exhaled breath, the method including:
providing exhaled breath capture module of claim 1;
capturing the one or more proteases present in the exhaled breath using the packed bed column, wherein the packed bed column is activated prior to capturing the non-volatile particles including proteases;
generating one or more collected liquid samples by extracting the non-volatile particles including proteases from the packed bed column using a solvent;
producing one or more reacted liquid samples associated with each of the one or more collected liquid samples by contacting a first aliquot of the one or more collected liquid samples with a plurality of substrate-based probes to disengage a cleavable molecule associated with each of the substrate-based probes in the plurality of substrate-based probes in response to proteolysis activity associated with the interaction between each of the substrate-based probes and the proteases;
estimating a concentration of the proteases in the one or more reacted liquid samples by analyzing the one or more reacted liquid samples using MALDI-TOFMS;
examining if the concentration of the one or more proteases in the one or more reacted liquid samples is equal to or greater than a cut-off threshold concentration corresponding to the proteases, wherein the cut-off threshold concentration is associated with a lower respiratory tract infection (LRTI); and
identifying a causative pathogen associated with the LRTI by analyzing a second aliquot of the one or more collected liquid samples using quantitative polymerase chain reaction (qPCR).

18. The method of claim 17, wherein the cut-off threshold concentration associated with the proteases is between about 0.15 picomol (pM) and about 0.5 pM.

19. The method of claim 17, wherein the exhaled breath is collected from a patient inhaling supplemental oxygen supplied via a cannula inserted into the patient's nostrils, allowing the patient to inhale the supplemental oxygen while exhaling into the mouthpiece associated with the exhaled breath capture module.

20. The method of claim 17, wherein the plurality of substrate-based probes includes PEG36-Nle(O-Bzl)-Met(O)2-Oic-Abu-ACC, PEG20-PEG20-PLGLKARR, or PEG25-PEG20-KPLGLKAR.

21. The method of claim 17, wherein the proteases include one or more of human neutrophil elastase, trypsinogen, serine protease 22, Kallikreins, or matrix metalloproteinases (MMPs).

22. The method of claim 17, wherein the solvent includes one or more of acetonitrile (ACN), methanol, trifluoro acetic acid (TFA), or isopropanol (IPA), the remaining being water.

23. The method of claim 17, wherein the solvent includes between about 50 vol.-% and about 70 vol.-% acetonitrile in water, between about 50 vol.-% and about 70 vol.-% isopropanol in water, and between about 0.05 vol.-% TFA in water.
